(19) United States
(12) Patent Application Publication
(10) Pub. No.: US 2024/0099973 A1
(43) Pub. Date: Mar. 28, 2024

(54) EXTRACELLULAR VESICLE COMPOSITIONS

(71) Applicant: LONZA SALES AG, BASEL (CH)

(72) Inventors: Conlin O'NEIL, Cambridge, MA (US); Raymond BOURDEAU, Cambridge, MA (US)

(73) Assignee: LONZA SALES AG, BASEL (CH)

(21) Appl. No.: 17/763,996

(22) PCT Filed: Sep. 25, 2020
(86) PCT No.: PCT/US2020/052935

§ 371 Date: Oct. 4, 2023

Related U.S. Application Data
(60) Provisional application No. 62/906,018, filed on Sep. 25, 2019.
(60) Provisional application No. 62/906,485, filed on Sep. 26, 2019.

(51) Int. Cl. A61K 9/127; A61K 31/7088; A61K 38/20; A61K 47/02; A61K 47/26; A61K 47/69

(52) U.S. Cl. CPC A61K 9/127; A61K 31/7088; A61K 38/208; A61K 47/02; A61K 47/26; A61K 47/6911

(57) ABSTRACT

The present disclosure relates to compositions for the storage and administration of extracellular vesicles (e.g., exosomes) that can comprise a scaffold protein and one or more (e.g., 1, 2, 3, 4, 5 or more) exogenous biologically active moieties. Also provided herein are methods for producing the exosomes and methods for using the exosomes to treat and/or prevent a range of medical disorders.

### CROSS REFERENCE TO RELATED APPLICATIONS

This PCT application claims the priority benefit of U.S. Provisional Application Nos. 62/906,018 filed Sep. 25, 2019; and 62/906,485 filed Sep. 26, 2019; each of which is incorporated herein by reference in its entirety.

### REFERENCE TO SEQUENCE LISTING SUBMITTED ELECTRONICALLY VIA EFS-WEB

The content of the electronically submitted sequence listing (Name: 4000_069PC02_Seglisting_ST25, Size: 68,911 bytes; and Date of Creation: Sep. 24, 2020) submitted in this application is incorporated herein by reference in its entirety.

### FIELD OF DISCLOSURE

The present disclosure relates to compositions for the storage and administration of extracellular vesicles (EVs), e.g., exosomes, that can comprise one or more exogenous biologically active moieties, and methods of preparing and using such compositions.

### BACKGROUND

EVs, e.g., exosomes, are important mediators of intercellular communication. They are also important biomarkers in the diagnosis and prognosis of many diseases, including cancer. As drug delivery vehicles, EVs offer advantages over traditional drug delivery methods (e.g., peptide immunization, DNA vaccines) as a new treatment modality in many therapeutic areas. One area of research is the formulation of compositions which can stably comprise EVs during lengthy storage periods prior to patient administration, without compromising the efficacy of the EVs. Known formulations suffer from drawbacks. For example, certain formulations e.g., those containing TRIS buffer, do not prevent the pH from fluctuating at various temperatures (i.e., when the formulation is frozen or thawed). Even small variations in pH can induce aggregation of EVs, thereby reducing or preventing their functionality. Further, known compositions include extraneous components such as exogenously added polypeptides, e.g., human serum albumin, or chelating agents.

Accordingly, there is a need for effective compositions for the storage and administration of EVs which overcome the drawbacks of known formulations, and can thus better enable the therapeutic use and other applications of EV-based technologies.

### BRIEF SUMMARY OF THE DISCLOSURE

Provided herein are compositions for the storage and administration of extracellular vesicles, e.g., exosomes. The compositions of the present disclosure provides reduced aggregation of EVs, improved stability of EVs, improved integrity of EV architecture, improved stability of engineered proteins contained on or in EVs, and improved stability of passively loaded or conjugated materials such as small molecule drugs or proteins. Such compositions are capable of being frozen, stored at a range of temperatures over various lengths of time, and thawed, without compromising the stability of the EVs contained within the composition. The EVs of the present disclosure can include biologically active moieties, such that the compositions can be used to treat a plurality of diseases or conditions where administration of EVs, e.g., EVs modified to include the biologically active moieties disclosed herein, are of beneficial effect to a subject.

In some aspects, the present disclosure provides a pharmaceutical composition comprising (a) an extracellular vesicle; (b) a saccharide; (c) sodium chloride; (d) a potassium phosphate; and (e) a sodium phosphate, wherein the composition is in a solution at a pH of about 7.2. In some aspects, the extracellular vesicle is an exosome.

In some aspects, the composition is capable of being stored for at least about 4 hours, at least about 5 hours, at least about 6 hours, at least about 7 hours, at least about 8 hours, at least about 9 hours, at least about 10 hours, at least about 11 hours, at least about 12 hours, at least about 15 hours, at least about 20 hours, at least about 24 hours, at least about 2 days, at least about 3 days, at least about 4 days, at least about 5 days, at least about 6 days, or at least about 7 days at a temperature of 4° C.

In some aspects, composition is capable of being frozen and thawed, wherein the thawed composition has a pH of about 7.2. In some aspects, the composition has a pH of 7.0, 7.1, 7.2, 7.3, or 7.4.

In some aspects, the pI is in the range of about 1 to about 6.5.

In some aspects, the composition has (i) reduced aggregates, (ii) improved stability of the EV, (iii) improved integrity of the EV architecture, (iv) improved stability of engineered proteins contained on or in EVs, and (v) improved stability of passively loaded or conjugated materials such as small molecule drugs or proteins.

In some aspects, the saccharide comprises a monosaccharide, a disaccharide, a trisaccharide, an oligosaccharide, a polysaccharide, a sugar alcohol, or any combination thereof. In some aspects, the saccharide has a molecular weight of from about 340.00 g/mol to about 380.00 g/mol. In some aspects, the saccharide comprises lactose, glucose, sucrose, trehalose, dextrose, and/or combinations thereof. In some aspects, the saccharide is a sugar alcohol having a molecular weight of from about 90.00 g/mol to about 190.00 g/mol. In some aspects, the sugar alcohol comprises glycerol, sorbitol, mannitol, xylitol, and/or combinations thereof. In some aspects, the saccharide is a sucrose or a trehalose. In some aspects, the saccharide is present in the composition at a concentration of about 5% w/v.

In some aspects, the present disclosure provides a pharmaceutical composition comprising (i) an extracellular vesicle and (ii) a saccharide, which is a sucrose or a trehalose at a concentration of about 5% w/v. In some aspects, the composition has improved stability compared to a reference composition comprising a sucrose or a trehalose at a concentration of 1% w/v to 4% w/v.

In some aspects, the composition has a conductivity between about 6 mS/cm and about 10 mS/cm. In some aspects, the conductivity is between 6 mS/cm and about 7 mS/cm, between about 7 mS/cm and about 8 mS/cm, between about 8 mS/cm and about 9 mS/cm, or between about 9 mS/cm and about 10 mS/cm. In some aspects, the conductivity is about 6 mS/cm, about 7 mS/cm, about 8 mS/cm, about 9 mS/cm, or about 10 mS/cm.

In some aspects, the composition further comprises sodium chloride. In some aspects, the sodium chloride is present in the composition at a concentration of between about 10 mM and about 134 mM. In some aspects, the concentration of sodium chloride is between about 10 mM to about 130 mM, between about 20 mM to about 120 mM, between about 30 mM to about 110 mM, between about 40 mM to about 100 mM, between about 50 mM to about 90 mM, between about 60 mM to about 80 mM, between about 70 mM to about 80 mM, between about 45 mM to about 95 mM, between about 45 mM to about 80 mM, between about 45 mM to about 70 mM, between about 45 mM to about 65 mM, between about 50 mM to about 65 mM, between about 50 mM to about 60 mM, between about 50 mM to about 55 mM, between about 50 mM to about 55 mM, or between about 51 mM to about 54 mM. In some aspects, the concentration of sodium chloride is about 10 mM, about 20 mM, about 30 mM, about 40 mM, about 50 mM, about 60 mM, about 70 mM, about 80 mM, about 90 mM, or about 100 mM. In some aspects, the concentration of sodium chloride is about 39 mM, about 40 mM, about 41 mM, about 42 mM, about 43 mM, about 44 mM, about 45 mM, about 46 mM, about 47 mM, about 48 mM, about 49 mM, or about 50 mM.

In some aspects, the composition further comprises a phosphate buffer. In some aspects, the phosphate buffer comprises at least one phosphate compound comprising potassium phosphate, sodium phosphate, disodium hydrogen phosphate, potassium dihydrogen phosphate, dipotassium phosphate, and/or a combination thereof.

In some aspects, the phosphate buffer comprises a potassium phosphate and a sodium phosphate at a ratio of about 1:about 2, about 1:about 3, about 1:about 4; or about 1: about 5. In some aspects, the phosphate buffer comprises a potassium phosphate and a sodium phosphate at a ratio of about 1:about 3. In some aspects, the phosphate buffer comprises a potassium phosphate and a sodium phosphate at a ratio of about 1:about 2.

In some aspects, the present disclosure provides a pharmaceutical composition comprising (i) an extracellular vesicle, (ii) a potassium phosphate, and (iii) a sodium phosphate in a solution, wherein the ratio of the potassium phosphate and the sodium phosphate is about 1 to about 3 or about 1 to about 2.

In some aspects, the solution has a pH of 7.1 to 7.3.

In some aspects, the potassium phosphate is present in the composition at a concentration of between about 1 mM to about 20 mM, between about 2 mM to about 19 mM, between about 3 mM to about 18 mM, between about 4 mM to about 17 mM, between about 5 mM to about 16 mM, or between about 5 mM to about 15 mM.

In some aspects, the concentration of the potassium phosphate is about 4.5 mM, about 4.6 mM, about 4.7 mM, about 4.8 mM, about 4.9 mM, about 5.0 mM, about 5.1 mM, about 5.2 mM, about 5.3 mM, about 5.4 mM, or about 5.5 mM. In some aspects, the concentration of the potassium phosphate is 5.15 mM.

In some aspects, the concentration of the potassium phosphate is about 15.0 mM, about 15.1 mM, about 15.2 mM, about 15.3 mM, about 15.4 mM, about 15.5 mM, about 15.6 mM, about 15.7 mM, about 15.8 mM, about 15.9 mM, about 16.0, about 16.1 mM, about 16.2 mM, about 16.3 mM, about 16.4 mM, or about 16.5 mM. In some aspects, the concentration of the potassium phosphate is 15.4 mM.

In some aspects, the potassium phosphate is potassium phosphate monobasic.

In some aspects, the sodium phosphate is present in the composition at a concentration of between about 10 mM to about 30, between about 11 mM to about 29 mM, between about 12 mM to about 28 mM, between about 13 mM to about 27 mM, or between about 14 mM to about 26 mM.

In some aspects, the composition of claim 39, wherein the sodium phosphate is present in the composition at a concentration of about 14.5 mM, about 14.6 mM, about 14.7 mM, about 14.8 mM, about 14.9 mM, about 15.0 mM, about 15.1, mM about 15.2, mM, about 15.3 mM, about 15.4, mM, or about 15.5 mM.

In some aspects, the concentration of the sodium phosphate is 14.9 mM.

In some aspects, the sodium phosphate is present in the composition at a concentration of about 26.5 mM, about 26.6 mM, about 26.7 mM, about 26.8 mM, about 26.9 mM, about 27.0 mM, about 27.1, mM about 27.2, mM, about 27.3 mM, about 27.4, mM, or about 27.5 mM. In some aspects, the concentration of the sodium phosphate is 27.1 mM.

In some aspects, the sodium phosphate is sodium phosphate dibasic heptahydrate.

In some aspects, the composition further comprises an anti-oxidant. In some aspects, the anti-oxidant comprises D-methionine, L-methionine. ascorbic acid, erythorbic acid, Na ascorbate, thioglycerol, cysteine, acetylcysteine, cystine, dithioerythreitol, glutathione, tocopherols, butylated hydroxyanisole (BHA), butylated hydroxytoluene (BHT), sodium bisulphate, sodium dithionite, A-Tocopherol, γ-Tocopherol, propyl gallate, ascorbyl palmitate, sodium metabisulfite, thiourea, sodium thiosulfate, propyl gallate, and sodium thioglycolate

In some aspects, the composition is not lyophilized.

In some aspects, the composition does not comprise a chelating agent.

In some aspects, the composition does not comprise albumin.

In some aspects, the present disclosure provides a composition comprising (a) a sucrose at a concentration of about 5% w/v, (b) sodium chloride at a concentration of about 50 mM; (c) a potassium phosphate monobasic at a concentration of about 5 mM; and (d) a sodium phosphate dibasic heptahydrate at a concentration of about 15 mM; wherein the composition is in a solution at a pH of 7.2 and at a conductivity of 8.8 mS/cm.

In some aspects, the present disclosure provides a composition comprising (a) a sucrose at a concentration of about 5% w/v, (b) sodium chloride at a concentration of about 40 mM; (c) a potassium phosphate monobasic at a concentration of about 15 mM; and (d) a sodium phosphate dibasic heptahydrate at a concentration of about 27 mM; wherein the composition is in a solution at a pH of 7.2 and at a conductivity of 7.2 mS/cm.

In some aspects, the composition is capable of being stored at a temperature of from about −20° C. to about −80° C., wherein the stability of the extracellular vesicle is not reduced. In some aspects, the composition can be stored for about one week, about two weeks, about three weeks, about four weeks, about one month, about two months, about three months, about four months, about five months, about six months, about seven months, about eight months, about nine months, about ten months, about 11 months, about 12 months, about one year, about two years, about three years, about four years, or about five years.

In some aspects, the extracellular vesicle is an exosome.

In some aspects, the extracellular vesicle further comprises a scaffold protein. In some aspects, the scaffold protein is Scaffold X. In some aspects, a payload are linked to the scaffold protein. In some aspects, the payload is linked to the scaffold protein by a linker. In some aspects, wherein the linker is a polypeptide. In some aspects, the linker is a non-polypeptide moiety. In some aspects, Scaffold X is a scaffold protein that is capable of anchoring the payload on the exterior surface of the extracellular vesicle.

In some aspects, the scaffold protein comprises prostaglandin F2 receptor negative regulator (the PTGFRN protein).

In some aspects, the scaffold protein comprises the PTGFRN protein or a fragment thereof. In some aspects, the scaffold protein comprises an amino acid sequence as set forth in any one of SEQ ID NOs: 1-7 and 33. In some aspects, the scaffold protein comprises an amino acid sequence at least 50%, at least 60%, at least 70%, at least 80%, at least 85%, at least 90%, at least 95%, at least 96%, at least 97%, at least 98%, at least 99%, or about 100% identical to SEQ ID NO: 1.

In some aspects, the extracellular vesicle comprises a biologically active moiety. In some aspects, the scaffold protein is fused to the biologically active moiety. In some aspects, the scaffold protein is not fused to the biologically active moiety.

In some aspects, the biologically active moiety comprises a STING agonist. In some aspects, the STING agonist comprises CL656. In some aspects, the STING agonist is isomer A, isomer B, isomer C, or isomer D of CL656. In some aspects, the STING agonist is not fused to the scaffold protein.

In some aspects, the biologically active moiety is IL-12. In some aspects, the biologically active moiety is CD40L. In other aspects, the biologically active moiety is FTL3L.

In some aspects, the biologically active moiety is fused to the scaffold protein.

In some aspects, the composition can be administered by a parenteral, topical, intravenous, oral, subcutaneous, intra-arterial, intradermal, transdermal, rectal, intracranial, intraperitoneal, intranasal, intratumoral, intramuscular route, or as an inhalant.

In some aspects, the present disclosure provides a method of preparing a pharmaceutical composition comprising combining: (a) an extracellular vesicle; (b) a saccharide; (c) sodium chloride; (d) a potassium phosphate; and (e) a sodium phosphate. In some aspects, the extracellular vesicle is an exosome.

In some aspects, the present disclosure provides a method of preparing a pharmaceutical composition comprising combining an extracellular vesicle and a saccharide, which is a sucrose or trehalose at a concentration of about 5% w/v, wherein the composition exhibits improved stability compared to a composition comprising sucrose or trehalose at a concentration of 1 to 4%.

In some aspects, the present disclosure provides a method of preparing a pharmaceutical composition comprising combining an extracellular vesicle and a phosphate compound, wherein the phosphate compound comprises a potassium phosphate and a sodium phosphate at a ratio that gives rise to a pH between 7.1 and 7.3.

In some aspects, the conductivity of the composition can be adjusted. In some aspects, the conductivity of the composition is between about 7.1 to about 7.3 mS/cm. In some aspects, the present disclosure provides the conductivity is 7.23 mS/cm.

In some aspects, the present disclosure provides a method of treating a disease or a condition in a subject in need thereof comprising administering to the subject the composition disclosed herein. In some aspects, the disease or condition is a cancer, a fibrosis, a hemophilia, diabetes, a growth factor deficiency, an eye disease, a Pompe disease, a lysosomal storage disorder, mucovicidosis, cystic fibrosis, Duchenne and Becker muscular dystrophy, transthyretin amyloidosis, hemophilia A, hemophilia B, adenosine-deaminase deficiency, Leber's congenital amaurosis, X-linked adrenoleukodystrophy, metachromatic leukodystrophy, OTC deficiency, glycogen storage disease 1A, Criggler-Najjar syndrome, primary hyperoxaluria type 1, acute intermittent porphyria, phenylketonuria, familial hypercholesterolemia, mucopolysaccharidosis type VI, α1 antitrypsin deficiency, and a hypercholesterolemia. In some aspects, the cancer is bladder cancer, cervical cancer, renal cell cancer, testicular cancer, colorectal cancer, lung cancer, head and neck cancer, ovarian, lymphoma, liver cancer, glioblastoma, melanoma, myeloma, leukemia, pancreatic cancer, or combinations thereof.

In some aspects, the present disclosure provides a pharmaceutical composition for treating a disease or a condition in a subject in need thereof.

In some aspects, the present disclosure provides a use of the composition disclosed herein in the manufacture of a medicament for treating a disease or a condition.

### BRIEF DESCRIPTION OF FIGURES

FIG. 1A is a schematic drawing of an exosome containing Protein X and a biologically active moiety, according to an aspect of the disclosure. Surface proteins CD9, CD81, and TSG101, and a sphingomyelin lipid domain are shown. Exosomal components mRNA, miRNA, and metabolites are further shown.

FIG. 1B is a graph showing the zeta potential (in mV) of native (black squares) and Protein X-containing exosomes (circles), according to an aspect of the disclosure. The X-axis is the pH. The Y-axis is the zeta-potential in mV.

FIG. 2A is a graph showing the Z-average (in nm) of native exosomes (black square) and Protein X exosomes (circle), at different pH values, according to an aspect of the disclosure. The X-axis is the pH. The Y-axis is the Z-average in nm.

FIG. 2B is a schematic drawing showing the stable pH range (rounded square) of EVs, according to an aspect of the disclosure. The stable pH range shown is between 7 and 8. The arrows indicate the result of pH variations on the EV if the pH is too low (EV aggregation) or too high (lipid hydrolysis).

FIG. 2C is an enlarged image of a cryogenic transmission electron micrograph showing a Protein X EV, according to an aspect of the disclosure. The exosomes were vitrified in water, not dried. This image shows the exosomes in water without ice crystals. The theoretical isoelectric point (pI) of the Protein X EV is noted as 6.2. The size of the protein X-containing EV is noted as ˜100 nm.

FIG. 2D is a photograph of microcentrifuge tubes containing various compositions, according to aspects of the disclosure. Tube A contains sucrose+buffer which has been frozen and thawed three times. Tube B contains buffer only, which has been frozen and thawed three times. Tube C contains Milli-Q water only, which has been frozen and thawed three times. The tubes show the presence of both color change and turbidity. Also shown is that the salt offers some degree of protection compared of the pure water sample.

FIG. 3A is a graph showing the distribution of STING agonist concentration (in μM) in exosomes as compared to the STING agonist in supernatants, with exosome-containing buffers stored at temperatures of −80° C., 4° C., and 22° C., for a period of between 0 and 12 hours, according to an aspect of the disclosure. This graph shows the 0-12 hour time points of FIG. 3B. The X-axis is the time in hours. The Y-axis is the concentration of STING agonist in μM.

FIG. 3B is a graph showing the distribution of STING agonist concentration (in μM) in exosomes as compared to the STING agonist in supernatants, with exosome-containing buffers stored at temperatures of −80° C., 4° C., and 22° C., for a period of between 12 and 72 hours, according to an aspect of the disclosure. The X-axis is the time in hours. The Y-axis is the concentration of STING agonist in μM.

FIG. 3C is a bar graph showing the effect of administration of phosphate buffered saline, native exosomes in buffer, and exosomes containing a STING agonist in buffer on gene expression in C57BL/6 mice, at 4 hours after administration, according to an aspect of the disclosure. The exosome-containing buffers were stored at 4° C. and 22° C. for 24 or 72 hours. The X-axis is the different tested articles. The Y-axis is the level of normalized gene expression.

FIG. 3D is a graph showing the intratumoral concentration of free STING agonist (dashed line, bottom) and exosome-encapsulated STING agonist (solid line, top) in B16-F10 melanoma tumors of C57BL/6 mice over time, according to an aspect of the disclosure. The X-axis is the time in minutes. The Y-axis is the concentration of STING agonist in nM.

FIGS. 4A-4C are schematic drawings of the process for preparing STING agonists encapsulated in exosomes according to an aspect of the disclosure. FIG. 4A shows loading of STING agonists into an exosome by saturating the buffer solution containing the exosomes with STING agonist, such that a portion of the STING diffuses into the exosome. FIG. 4B shows purification of the exosomes (i.e., removal of STING agonist that did not diffuse into the exosomes). FIG. 4C shows equilibration of the STING agonist-containing exosomes (i.e., passive diffusion of STING agonists out of the exosomes into the surrounding buffer).

FIG. 5 is a graph showing IL-12 content (in ng/mL) associated with exosomes. The X-axis is the amount of time (in days) the exosomes are stored. The Y-axis is the content of IL-12 in ng/mL. From left to right, the bars represent (1) control, no peroxide; (2) control, 0.05% peroxide; (3) thiosulfate, no peroxide; (4) thiosulfate, 0.05% peroxide; (5) ascorbate, no peroxide; (6) ascorbate, 0.05% peroxide; (7) glutathione, no peroxide; (8) glutathione, 0.05% peroxide; (9) methionine, no peroxide; (10) methionine, 0.05% peroxide.

FIG. 6 is a graph showing the diameter (in nm) of exosomes, after different lengths of time. The X-axis is the amount of time (in days) the exosomes are stored. The Y-axis is the diameter of the exosomes (in nm). From left to right, the bars represent (1) control; (2) control and hydrogen peroxide; (3) thiosulfate; (4) thiosulfate and hydrogen peroxide; (5) ascorbate; (6) ascorbate and hydrogen peroxide; (7) glutathione; (8) glutathione and hydrogen peroxide; (9) methionine; (10) methionine and hydrogen peroxide.

FIG. 7 is a graph showing PDI of an exosome solution, after different lengths of time. The X-axis is the amount of time (in days) the exosomes are stored. The Y-axis polydispersity index.

### DETAILED DESCRIPTION OF DISCLOSURE

The present disclosure is directed to compositions for the storage and administration of EVs, e.g., exosomes, which can comprise one or more biologically active moieties in which the stability and integrity of the EVs and payloads are maintained through various time periods and temperatures, including freezing and thawing. Multiple biologically active moieties can be attached (or linked) to one or more scaffold moieties on the exterior surface of EVs (e.g., exosomes). Non-limiting examples of the various aspects are disclosed herein.

### I. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “extracellular vesicle” or “EV” refers to a cell-derived vesicle comprising a membrane that encloses an internal space. Extracellular vesicles comprise all membrane-bound vesicles (e.g., exosomes, nanovesicles) that have a smaller diameter than the cell from which they are derived. In some aspects, extracellular vesicles range in diameter from 20 nm to 1000 nm, and can comprise various macromolecular payloads either within the internal space (i.e., lumen), displayed on the external surface of the extracellular vesicle, and/or spanning the membrane. In some aspects, the payload can comprise nucleic acids, proteins, carbohydrates, lipids, small molecules, and/or combinations thereof. In some aspects, an EV comprises multiple (e.g., two or more) payloads or other exogenous biologically active moieties. In certain aspects, an extracellular vehicle can further comprise one or more scaffold moieties. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, prokaryotic or eukaryotic cells, and/or cultured cells. In some aspects, the extracellular vesicles are produced by cells that express one or more transgene products. The EVs disclosed herein have been modified and therefore, do not comprise naturally occurring EVs.

As used herein, the term “exosome” refers to an extracellular vesicle with a diameter between 20-300 nm (e.g., between 40-200 nm). Exosomes comprise a membrane that encloses an internal space (i.e., lumen), and, in some aspects, can be generated from a cell (e.g., producer cell) by direct plasma membrane budding or by fusion of the late endosome with the plasma membrane. In some aspects, an exosome comprises multiple (e.g., two or more) exogenous biologically active moieties (e.g., as described herein). In certain aspects, an exosome further comprises one or more scaffold moieties. As described infra, exosomes can be derived from a producer cell, and isolated from the producer cell based on its size, density, biochemical parameters, or a combination thereof. In some aspects, the EVs (e.g., exosomes) of the present disclosure are produced by cells that express one or more transgene products. The exosomes of the present disclosure are modified and therefore, do not comprise naturally occurring exosomes.

As used herein, the term “nanovesicle” refers to an extracellular vesicle with a diameter between 20-250 nm (e.g., between 30-150 nm) and is generated from a cell (e.g., producer cell) by direct or indirect manipulation such that the nanovesicle would not be produced by the cell without the manipulation. Appropriate manipulations of the cell to produce the nanovesicles include but are not limited to serial extrusion, treatment with alkaline solutions, sonication, or combinations thereof. In some aspects, production of nanovesicles can result in the destruction of the producer cell. In some aspects, population of nanovesicles described herein are substantially free of vesicles that are derived from cells by way of direct budding from the plasma membrane or fusion of the late endosome with the plasma membrane. In some aspects, a nanovesicle comprises multiple (e.g., at least two) exogenous biologically active moieties. In certain aspects, a nanovesicle further comprises one or more scaffold moieties. Nanovesicles, once derived from a producer cell, can be isolated from the producer cell based on its size, density, biochemical parameters, or a combination thereof. As used herein, nanovesicles have been modified and therefore, do not comprise naturally occurring nanovesicles.

As used herein the term “surface-engineered EVs, e.g., exosomes” (e.g., Scaffold X-engineered EVs, e.g., exosomes) refers to an EV (e.g., exosome) with the membrane or the surface modified in its composition, so that the membrane or the surface of the engineered EV (e.g., exosome), is different from either that of the EV prior to the modification or of the naturally occurring EV. The engineering can be on the surface of the EV (e.g., exosome) or in the membrane of the EV (e.g., exosome) so that the surface of the EV, e.g., exosome, is changed. For example, the membrane is modified in its composition of a protein, a lipid, a small molecule, a carbohydrate, etc. The composition can be changed by a chemical, a physical, or a biological method or by being produced from a cell previously or concurrently modified by a chemical, a physical, or a biological method. Specifically, the composition can be changed by genetic engineering or by being produced from a cell previously modified by genetic engineering. In some aspects, a surface-engineered EV, e.g., exosome, comprises multiple (e.g., at least two) exogenous biologically active moieties. In certain aspects, the exogenous biologically active moieties can comprise an exogenous protein (i.e., a protein that the EV, e.g., exosome, does not naturally express) or a fragment or variant thereof that can be exposed to the surface of the EV, e.g., exosome, or can be an anchoring point (attachment) for a moiety exposed on the surface of the EV, e.g., exosome. In other aspects, a surface-engineered EV, e.g., exosome, comprises a higher expression (e.g., higher number) of a natural exosome protein (e.g., Scaffold X) or a fragment or variant thereof that can be exposed to the surface of the EV, e.g., exosome, or can be an anchoring point (attachment) for a moiety exposed on the surface of the EV, e.g., exosome.

The term “modified,” when used in the context of EVs, e.g., exosomes described herein, refers to an alteration or engineering of an EV, e.g., exosome and/or its producer cell, such that the modified EV, e.g., exosome is different from a naturally-occurring EV, e.g., exosome. In some aspects, a modified EV, e.g., exosome described herein comprises a membrane that differs in composition of a protein, a lipid, a small molecular, a carbohydrate, etc. compared to the membrane of a naturally-occurring EV, e.g., exosome (e.g., membrane comprises higher density or number of natural exosome proteins and/or membrane comprises multiple (e.g., at least two) biologically active moieties that are not naturally found in exosomes. As used herein, biologically active moieties that are not naturally found in exosomes are also described as “exogenous biologically active moieties.” In certain aspects, such modifications to the membrane changes the exterior surface of the EV, e.g., exosome (e.g., surface-engineered EVs, e.g., exosomes described herein).

As used herein, the term “scaffold moiety” refers to a molecule that can be used to anchor a payload or any other exogenous biologically active moiety of interest to the EV, e.g., exosome, either on the luminal surface or on the exterior surface of the EV, e.g., exosome. In certain aspects, a scaffold moiety comprises a synthetic molecule. In some aspects, a scaffold moiety comprises a non-polypeptide moiety. In other aspects, a scaffold moiety comprises a lipid, carbohydrate, or protein that naturally exists in the EV, e.g., exosome. In some aspects, a scaffold moiety comprises a lipid, carbohydrate, or protein that does not naturally exist in the EV, e.g., exosome. In certain aspects, a scaffold moiety is Scaffold X. In further aspects, a scaffold moiety comprises a Scaffold X and another scaffold moiety. Non-limiting examples of other scaffold moieties that can be used with the present disclosure include: aminopeptidase N (CD13); Neprilysin, AKA membrane metalloendopeptidase (MME); ectonucleotide pyrophosphatase/phosphodiesterase family member 1 (ENPP1); Neuropilin-1 (NRP1); CD9, CD63, CD81, PDGFR, GPI anchor proteins, lactadherin, LAMP2, and LAMP2B.

As used herein, the term “Scaffold X” refers to exosome proteins that have recently been identified on the surface of exosomes. See, e.g., U.S. Pat. No. 10,195,290, which is incorporated herein by reference in its entirety. Non-limiting examples of Scaffold X proteins include: prostaglandin F2 receptor negative regulator (“the PTGFRN protein”); basigin (“the BSG protein”); immunoglobulin superfamily member 2 (“the IGSF2 protein”); immunoglobulin superfamily member 3 (“the IGSF3 protein”); immunoglobulin superfamily member 8 (“the IGSF8 protein”); integrin beta-1 (“the ITGB1 protein); integrin alpha-4 (“the ITGA4 protein”); 4F2 cell-surface antigen heavy chain (“the SLC3A2 protein”); and a class of ATP transporter proteins (“the ATP1A1 protein,” “the ATP1A2 protein,” “the ATP1A3 protein,” “the ATP1A4 protein,” “the ATP1B3 protein,” “the ATP2B1 protein,” “the ATP2B2 protein,” “the ATP2B3 protein,” “the ATP2B protein”). In some aspects, a Scaffold X protein can be a whole protein or a fragment thereof (e.g., functional fragment, e.g., the smallest fragment that is capable of anchoring another moiety on the exterior surface or on the luminal surface of the EV, e.g., exosome). In some aspects, a Scaffold X can anchor a moiety (e.g., a payload) to the external surface or the luminal surface of the exosome.

As used herein, the term “Scaffold Y” refers to exosome proteins that were newly identified within the luminal surface of exosomes. See, e.g., International Publication No. WO/2019/099942, which is incorporated herein by reference in its entirety. Non-limiting examples of Scaffold Y proteins include: myristoylated alanine rich Protein Kinase C substrate (“the MARCKS protein”); myristoylated alanine rich Protein Kinase C substrate like 1 (“the MARCKSL1 protein”); and brain acid soluble protein 1 (“the BASP1 protein”). In some aspects, a Scaffold Y protein can be a whole protein or a fragment thereof (e.g., functional fragment, e.g., the smallest fragment that is capable of anchoring a moiety on the luminal surface of the EVs, e.g., exosomes,). In some aspects, a Scaffold Y can anchor a moiety (e.g., a STING agonist and/or an IL-12 moiety) to the lumen of the EVs, e.g., exosomes.

As used herein, the term “fragment” of a protein (e.g., therapeutic protein, or Scaffold X) refers to an amino acid sequence of a protein that is shorter than the naturally-occurring sequence, N- and/or C-terminally deleted or any part of the protein deleted in comparison to the naturally occurring protein. As used herein, the term “functional fragment” refers to a protein fragment that retains protein function. Accordingly, in some aspects, a functional fragment of a Scaffold X protein retains the ability to anchor a moiety on the luminal surface or on the exterior surface of the EV, e.g., exosome. Whether a fragment is a functional fragment can be assessed by any known methods to determine the protein content of EVs, e.g., exosomes including Western Blots, FACS analysis and fusions of the fragments with autofluorescent proteins like, e.g., GFP. In certain aspects, a functional fragment of a Scaffold X protein retains at least about 50%, at least about 60%, at least about 70%, at least about 80%, at least about 90% or at least about 100% of the ability, e.g., an ability to anchor a moiety, of the naturally occurring Scaffold X protein.

As used herein, the term “variant” of a molecule (e.g., functional molecule, antigen, or Scaffold X) refers to a molecule that shares certain structural and functional identities with another molecule upon comparison by a method known in the art. For example, a variant of a protein can include a substitution, insertion, deletion, frameshift or rearrangement in another protein.

In some aspects, a variant of a Scaffold X comprises a variant having at least about 70% identity to the full-length, mature PTGFRN, BSG, IGSF2, IGSF3, IGSF8, ITGB1, ITGA4, SLC3A2, or ATP transporter proteins or a fragment (e.g., functional fragment) of the PTGFRN, BSG, IGSF2, IGSF3, IGSF8, ITGB1, ITGA4, SLC3A2, or ATP transporter proteins. In some aspects, variants or variants of fragments of PTGFRN share at least about 70%, at least about 80%, at least about 85%, at least about 90%, at least about 95%, at least about 96%, at least about 97%, at least about 98%, or at least about 99% sequence identity with PTGFRN according to SEQ ID NO: 1 or with a functional fragment thereof.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

The term “percent sequence identity” or “percent identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software both for online use and for download. Suitable software programs are available from various sources, and for alignment of both protein and nucleotide sequences. One suitable program to determine percent sequence identity is bl2seq, part of the BLAST suite of programs available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

The generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI.

It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

The polynucleotide variants can contain alterations in the coding regions, non-coding regions, or both. In one aspect, the polynucleotide variants contain alterations which produce silent substitutions, additions, or deletions, but do not alter the properties or activities of the encoded polypeptide. In another aspect, nucleotide variants are produced by silent substitutions due to the degeneracy of the genetic code. In other aspects, variants in which 5-10, 1-5, or 1-2 amino acids are substituted, deleted, or added in any combination. Polynucleotide variants can be produced for a variety of reasons, e.g., to optimize codon expression for a particular host (change codons in the human mRNA to others, e.g., a bacterial host such as E. coli).

Naturally occurring variants are called “allelic variants,” and refer to one of several alternate forms of a gene occupying a given locus on a chromosome of an organism (Genes II, Lewin, B., ed., John Wiley & Sons, New York (1985)). These allelic variants can vary at either the polynucleotide and/or polypeptide level and are included in the present disclosure. Alternatively, non-naturally occurring variants can be produced by mutagenesis techniques or by direct synthesis.

Using known methods of protein engineering and recombinant DNA technology, variants can be generated to improve or alter the characteristics of the polypeptides. For instance, one or more amino acids can be deleted from the N-terminus or C-terminus of the secreted protein without substantial loss of biological function. Ron et al., J. Biol. Chem. 268: 2984-2988 (1993), incorporated herein by reference in its entirety, reported variant KGF proteins having heparin binding activity even after deleting 3, 8, or 27 amino-terminal amino acid residues. Similarly, interferon gamma exhibited up to ten times higher activity after deleting 8-10 amino acid residues from the carboxy terminus of this protein. (Dobeli et al., J. Biotechnology 7:199-216 (1988), incorporated herein by reference in its entirety.)

Moreover, ample evidence demonstrates that variants often retain a biological activity similar to that of the naturally occurring protein. For example, Gayle and coworkers (J. Biol. Chem 268:22105-22111 (1993), incorporated herein by reference in its entirety) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See Abstract.) In fact, only 23 unique amino acid sequences, out of more than 3,500 nucleotide sequences examined, produced a protein that significantly differed in activity from wild-type.

As stated above, polypeptide variants include, e.g., modified polypeptides. Modifications include, e.g., acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation (Mei et al., Blood 116:270-79 (2010), which is incorporated herein by reference in its entirety), proteolytic processing, phosphorylation, prenylation, racemization, selenoylation, sulfation, transfer-RNA mediated addition of amino acids to proteins such as arginylation, and ubiquitination. In some aspects, Scaffold X is modified at any convenient location.

As used herein the terms “linked to,” “conjugated to,” and “anchored to” are used interchangeably and refer to a covalent or non-covalent bond formed between a first moiety and a second moiety, e.g., Scaffold X and an exogenous biologically active moiety, respectively, e.g., a scaffold moiety expressed in or on the extracellular vesicle and an antigen, e.g., Scaffold X (e.g., a PTGFRN protein), respectively, in the luminal surface of or on the external surface of the extracellular vesicle.

The term “encapsulated”, or grammatically different forms of the term (e.g., encapsulation, or encapsulating), refers to a status or process of having a first moiety (e.g., exogenous biologically active moiety, e.g., STING agonist) inside a second moiety (e.g., an EV, e.g., exosome) without chemically or physically linking the two moieties. In some aspects, the term “encapsulated” can be used interchangeably with “in the lumen of”. Non-limiting examples of encapsulating a first moiety (e.g., exogenous biologically active moiety, e.g., antigen, adjuvant, or immune modulator) into a second moiety (e.g., EVs, e.g., exosomes) are disclosed elsewhere herein.

As used herein, the term “producer cell” refers to a cell used for generating an EV, e.g., exosome. A producer cell can be a cell cultured in vitro, or in a cell in vivo. A producer cell includes, but not limited to, a cell known to be effective in generating EVs, e.g., exosomes, e.g., HEK293 cells, Chinese hamster ovary (CHO) cells, mesenchymal stem cells (MSCs), BJ human foreskin fibroblast cells, fHDF fibroblast cells, AGE.HN® neuronal precursor cells, CAP© amniocyte cells, adipose mesenchymal stem cells, RPTEC/TERT1 cells. In certain aspects, a producer cell is not an antigen-presenting cell. In some aspects, a producer cell is not a dendritic cell, a B cell, a mast cell, a macrophage, a neutrophil, Kupffer-Browicz cell, cell derived from any of these cells, or any combination thereof. In some aspects, the EVs, e.g., exosomes useful in the present disclosure do not carry an antigen on MHC class I or class II molecule exposed on the surface of the EV, e.g., exosome, but instead can carry an antigen in the lumen of the EV, e.g., exosome or on the surface of the EV, e.g., exosome by attachment to Scaffold X.

As used herein, the terms “isolate,” “isolated,” and “isolating” or “purify,” “purified,” and “purifying” as well as “extracted” and “extracting” are used interchangeably and refer to the state of a preparation (e.g., a plurality of known or unknown amount and/or concentration) of desired EVs, that have undergone one or more processes of purification, e.g., a selection or an enrichment of the desired EV preparation. In some aspects, isolating or purifying as used herein is the process of removing, partially removing (e.g., a fraction) of the EVs from a sample containing producer cells. In some aspects, an isolated EV composition has no detectable undesired activity or, alternatively, the level or amount of the undesired activity is at or below an acceptable level or amount. In other aspects, an isolated EV composition has an amount and/or concentration of desired EVs at or above an acceptable amount and/or concentration. In other aspects, the isolated EV composition is enriched as compared to the starting material (e.g., producer cell preparations) from which the composition is obtained. This enrichment can be by 10%, 20%, 30%, 40%, 50%, 60%, 70%, 80%, 90%, 95%, 96%, 97%, 98%, 99%, 99.9%, 99.99%, 99.999%, 99.9999%, or greater than 99.9999% as compared to the starting material. In some aspects, isolated EV preparations are substantially free of residual biological products. In some aspects, the isolated EV preparations are 100% free, 99% free, 98% free, 97% free, 96% free, 95% free, 94% free, 93% free, 92% free, 91% free, or 90% free of any contaminating biological matter. Residual biological products can include abiotic materials (including chemicals) or unwanted nucleic acids, proteins, lipids, or metabolites. Substantially free of residual biological products can also mean that the EV composition contains no detectable producer cells and that only EVs are detectable.

As used herein, the term “immune modulator” refers to an agent that acts on a target (e.g., a target cell) that is contacted with the extracellular vesicle, and regulates the immune system. Non-limiting examples of immune modulator that can be introduced into an EV (e.g., exosome) and/or a producer cell include agents such as, modulators of checkpoint inhibitors, ligands of checkpoint inhibitors, cytokines, derivatives thereof, or any combination thereof. The immune modulator can also include an agonist, an antagonist, an antibody, an antigen-binding fragment, a polynucleotide, such as siRNA, miRNA, lncRNA, mRNA, DNA, or a small molecule.

As used herein, the term “payload” refers to an agent that acts on a target (e.g., a target cell) that is contacted with the EV. Non-limiting examples of payload that can be included on the EV, e.g., exosome, are a biologically active molecule, e.g., a therapeutic molecule, an adjuvant, and/or an immune modulator. Payloads that can be introduced into an EV, e.g., exosome, and/or a producer cell include agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, and siRNA), amino acids (e.g., amino acids comprising a detectable moiety or a toxin or that disrupt translation), polypeptides (e.g., enzymes), lipids, carbohydrates, and small molecules (e.g., small molecule drugs and toxins). In certain aspects, a payload comprises an exogenous biologically active moiety (e.g., those disclosed herein). In some aspects, a payload comprises a targeting moiety. In some aspects, a payload is “passively loaded” onto or into an EV, e.g., exosome. As used herein, the term “passively loaded” refers to the association between an EV, e.g., exosome, and a payload present in the same solution. Under passive loading, the payload will become associated with the EV by, for example, natural diffusion and/or attraction.

As used herein, the term a “targeting moiety” refers to an agent that can modify the distribution of extracellular vesicles (e.g., exosomes, nanovesicles) in vivo or in vitro (e.g., in a mixed culture of cells of different varieties). The targeting moiety can be a biological molecule, such as a protein, a peptide, a lipid, or a carbohydrate, or a synthetic molecule. For example, the targeting moiety can be an antibody (e.g., anti-CD19 nanobody, anti-CD22 nanobody), a synthetic polymer (e.g., PEG), a natural ligand (e.g., CD40L, albumin), a recombinant protein (e.g., XTEN), but not limited thereto. In certain aspects, the targeting moiety is displayed on the surface of EVs. The targeting moiety can be displayed on the EV surface by being fused to a scaffold protein (e.g., Scaffold X) (e.g., as a genetically encoded fusion molecule). In some aspects, the targeting moiety can be displayed on the EV surface by chemical reaction attaching the targeting moiety to an EV surface molecule. A non-limiting example is PEGylation. In some aspects, EVs disclosed herein (e.g., exosomes) can further comprise a targeting moiety (in addition to a payload). In some aspects, a targeting moiety described above can be combined with a functional moiety, such as a small molecule (e.g., STING, ASO), a drug, and/or a therapeutic protein (e.g., anti-CD3/anti-CD19 antibodies, anti-mesothelin antibody/pro-apoptotic proteins).

As used herein, the term “biologically active moiety” refers to an agent that has activity in a biological system (e.g., a cell or a human subject), including, but not limited to a protein, polypeptide or peptide including, but not limited to, a structural protein, an enzyme, a cytokine (such as an interferon and/or an interleukin) an antibiotic, a polyclonal or monoclonal antibody, or an effective part thereof, such as an Fv fragment, which antibody or part thereof can be natural, synthetic or humanized, a peptide hormone, a receptor, a signaling molecule or other protein; a nucleic acid, as defined below, including, but not limited to, an oligonucleotide or modified oligonucleotide, an antisense oligonucleotide or modified antisense oligonucleotide, cDNA, genomic DNA, an artificial or natural chromosome (e.g. a yeast artificial chromosome) or a part thereof, RNA, including mRNA, tRNA, rRNA or a ribozyme, or a peptide nucleic acid (PNA); a virus or virus-like particles; a nucleotide or ribonucleotide or synthetic analogue thereof, which can be modified or unmodified; an amino acid or analogue thereof, which can be modified or unmodified; a non-peptide (e.g., steroid) hormone; a proteoglycan; a lipid; or a carbohydrate. In some aspects, antisense oligonucleotides include a phosphorodiamidate Morpholino oligomer (PMO) or a peptide-conjugated phosphorodiamidate morpholino oligomer (PPMO). In certain aspects, a biologically active moiety comprises a therapeutic molecule (e.g., an antigen), a targeting moiety (e.g., an antibody or an antigen-binding fragment thereof), an adjuvant, an immune modulator, or any combination thereof. In some aspects, the biologically active moiety comprises a macromolecule (e.g., a protein, an antibody, an enzyme, a peptide, DNA, RNA, or any combination thereof). In some aspects, the biologically active moiety comprises a small molecule (e.g., an antisense oligomer (ASO), an siRNA, STING, a pharmaceutical drug, or any combination thereof). In some aspects, the biologically active moieties are exogenous to the exosome, i.e., not naturally found in the exosome.

As used herein, the term “therapeutic molecule” refers to any molecule that can treat and/or prevent a disease or disorder in a subject (e.g., human subject). In some aspects, a therapeutic molecule comprises an antigen. As used herein, the term “antigen” refers to any agent that when introduced into a subject elicits an immune response (cellular or humoral) to itself.

As used herein, the term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)₂, Fab, Fab′, and F(ab′)₂, F(ab1)₂, Fv, dAb, and Fd fragments, diabodies, and antibody-related polypeptides. Antibody includes bispecific antibodies and multispecific antibodies so long as they exhibit the desired biological activity or function. In some aspects, the antibody or antigen-binding fragment thereof comprises a scFv, scFab, scFab-Fc, nanobody, or any combination thereof. In some aspects, the antibody or antigen-binding fragment thereof comprises an agonist antibody, a blocking antibody, a targeting antibody, a fragment thereof, or a combination thereof. In some aspects, the agonist antibody is a CD40L agonist. In some aspects, the blocking antibody binds a target protein selected from programmed death 1 (PD-1), programmed death ligand 1 (PD-L1), cytotoxic T-lymphocyte-associated protein 4, and any combination thereof. In some aspects, the targeting antibody binds a CD3 and/or CD19.

The terms “individual,” “subject,” “host,” and “patient,” are used interchangeably herein and refer to any mammalian subject for whom diagnosis, treatment, or therapy is desired, particularly humans. The compositions and methods described herein are applicable to both human therapy and veterinary applications. In some aspects, the subject is a mammal, and in other aspects the subject is a human. As used herein, a “mammalian subject” includes all mammals, including without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like).

As used herein, the term “substantially free” means that the sample comprising EVs, e.g., exosomes, comprise less than 10% of macromolecules by mass/volume (m/v) percentage concentration. Some fractions can contain less than 0.001%, less than 0.01%, less than 0.05%, less than 0.1%, less than 0.2%, less than 0.3%, less than 0.4%, less than 0.5%, less than 0.6%, less than 0.7%, less than 0.8%, less than 0.9%, less than 1%, less than 2%, less than 3%, less than 4%, less than 5%, less than 6%, less than 7%, less than 8%, less than 9%, or less than 10% (m/v) of macromolecules.

As used herein, the term “macromolecule” means nucleic acids, contaminant proteins, lipids, carbohydrates, metabolites, or a combination thereof.

As used herein, the term “conventional exosome protein” means a protein previously known to be enriched in exosomes, including but is not limited to CD9, CD63, CD81, PDGFR, GPI anchor proteins, lactadherin LAMP2, and LAMP2B, a fragment thereof, or a peptide that binds thereto.

“Administering,” as used herein, means to give a composition comprising an EV, e.g., exosome, disclosed herein to a subject via a pharmaceutically acceptable route. Routes of administration can be intravenous, e.g., intravenous injection and intravenous infusion. Additional routes of administration include, e.g., subcutaneous, intramuscular, oral, nasal, and pulmonary administration. EVs, e.g., exosomes can be administered as part of a pharmaceutical composition comprising at least one excipient.

An “immune response,” as used herein, refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4+ cell, a CD8+ T cell, or a Treg cell, or activation or inhibition of any other cell of the immune system, e.g., NK cell. Accordingly an immune response can comprise a humoral immune response (e.g., mediated by B-cells), cellular immune response (e.g., mediated by T cells), or both humoral and cellular immune responses. In some aspects, an immune response is an “inhibitory” immune response. An inhibitory immune response is an immune response that blocks or diminishes the effects of a stimulus (e.g., antigen). In certain aspects, the inhibitory immune response comprises the production of inhibitory antibodies against the stimulus. In some aspects, an immune response is a “stimulatory” immune response. A stimulatory immune response is an immune response that results in the generation of effectors cells (e.g., cytotoxic T lymphocytes) that can destroy and clear a target antigen (e.g., tumor antigen or viruses).

“Treat,” “treatment,” or “treating,” as used herein refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also include prophylaxis or prevention of a disease or condition or its symptoms thereof. In one aspect, the term “treating” or “treatment” means inducing an immune response in a subject against an antigen.

“Prevent” or “preventing,” as used herein, refers to decreasing or reducing the occurrence or severity of a particular outcome. In some aspects, preventing an outcome is achieved through prophylactic treatment.

### II. Pharmaceutical Compositions

Provided herein are compositions for the storage and administration of extracellular vesicles, e.g., exosomes. As noted above, the compositions of the present disclosure provide multiple advantages, including but not limited to: reduced aggregation of EVs, improved stability of EVs, and improved integrity of EV architecture, improved stability of engineered proteins contained on or in EVs, and improved stability of passively loaded or conjugated materials such as small molecule drugs or proteins. The compositions disclosed herein are capable of being frozen, stored at a range of temperatures over various lengths of time, and thawed, without compromising the stability of the EVs contained within the composition.

Various components and considerations with respect to formulating the compositions of the present disclosure are set forth below.

II.A. EV Compositions

The present disclosure provides a pharmaceutical composition comprising an extracellular vesicle, wherein the pharmaceutical composition is stable for freezing and/or storage and/or is suitable for administration in a mammal, e.g., human. Instability of a biologic during storage can be caused by aggregation, deamination, isomerization, hydrolysis, oxidation, and/or denaturation. These structural modifications can occur due to various different factors: the properties of the biologic and/or other factors including temperature, pH, and the ionic strength of the biologics and the elements formulated with the biologic.

In some aspects, the pharmaceutical composition of the present disclosure is formulated stable so that the composition does not require a chelating agent and/or albumin, e.g., recombinant human albumin.

Human albumin is the most ubiquitous protein in blood and is present at amounts around 40 g/L. Its role in blood is the shuttling of numerous smaller entities such as metals, hormones, fatty acids and toxins. However, it also makes up about 75% of the colloidal oncotic (or colloidal osmotic) pressure of blood and the single free cysteine of albumin (at position 34) makes up the majority of the reducing equivalents present in blood. All these properties are traits which are functional in employing albumin in formulation.

Albumin has historically been used in a range of different formulations. Originally, plasma sourced human serum albumin was employed, but there has been a shift in the industry towards the use of chemically defined (recombinant) human serum albumin. Recombinant products are advantageous due to factors such as: the absence of animal-derived products, certainty of supply, high purity, absence of host-derived proteases, high homogeneity, high free thiol content, absence of known or unknown human pathogens, batch-to-batch consistency and the presence of an established regulatory pathway.

Albumin in formulation has been reported to prevent: surface adsorption, aggregation, fibrillation, and oxidation and to improve: solubility, lyophilised cake formation, and/or dissolving properties of the API from lyophilised powder. Despite these known benefits, the present disclosure provides a stable albumin-free pharmaceutical composition comprising an extracellular vesicle.

Chelating agents are ingredients that bind with metal ions and play a crucial role in the stability and efficacy of pharmaceutical formulations. The process of chelation stabilizes metal ions by preventing them from chemically reacting with any other substances. The present composition can be characterized that it does not contain a chelating agent.

In some aspects, the compositions disclosed herein have a pI in the range of about 1 to about 6.5. The pI range of the presently disclosed EVs, where a surface macromolecule is over-expressed, e.g., PTGFRN, as disclosed herein, enables colloidally stable, anionic exosomes at physiological pH values. In some aspects, the surface molecule could be polypeptide, oligonucleotide, or carbohydrate. In some aspects, a pI above 6.5, can cause the EVs, e.g., exosomes to have either a neutral charge (unstable), or a cationic charge at useful pH ranges which could result in toxicity or limited biodistribution.

In some aspects, the compositions of the present disclosure are formulated in a liquid state and can be frozen for storage by way of reducing the temperature of the compositions to freezing and subfreezing temperatures. Freezing the compositions via dehydration or lyophilization is not contemplated. In some aspects, the composition is not lyophilized.

II.B. Composition I (pH)

In one aspect, the present disclosure provides a pH that can stably formulate an EV. The pH can be in a range of about 7.0 to about 7.4, e.g., about 7.1 to about 7.3, e.g., about 7.2. In some aspects, a pharmaceutical composition of the present disclosure comprises (a) an extracellular vesicle; (b) a saccharide; (c) sodium chloride; (d) a potassium phosphate; and (e) a sodium phosphate. In some aspects, the composition is in a solution at a pH of about 7.2. The EVs are disclosed elsewhere herein, the saccharide can be a monosaccharide, a disaccharide, a trisaccharide, or any other saccharides; sodium chloride is shown below; and the potassium and sodium phosphates are shown below.

In some aspects, the composition prior to freezing and after the freezing remains the same. For example, the composition prior to freezing and after the freezing has a pH of about 7.1. In some aspects, the composition prior to freezing and after the freezing has a pH of about 7.2. In some aspects, the composition prior to freezing and after the freezing has a pH of about 7.3. In some aspects, the composition prior to freezing and after the freezing has a pH of about 7.4.

In some aspects, the pH of the composition can be adjusted by modifying the concentration of phosphates. In some aspects, the pH of the composition can be adjusted by modifying the concentration of the potassium phosphate. In some aspects, the pH of the composition can be increased by adding or increasing the concentration of a potassium phosphate. In some aspects, the concentration of the potassium phosphate is higher than the concentration of the sodium phosphate.

In some aspects, the ratio of the monobasic and dibasic forms of sodium phosphate and potassium phosphate can be used to adjust the pH of a pharmaceutical composition. In some aspects, sodium phosphate monobasic and/or potassium phosphate monobasic can be used to increase the pH of a pharmaceutical composition. In some aspects, sodium phosphate dibasic and/or potassium phosphate dibasic can be used to decrease the pH of a pharmaceutical composition. in some aspects, pH ranges for the disclosed compositions are between about 6.8 to about 7.6. Therefore, if the pH of the composition is lower than desired, the pH can be changed by changing the ratio of monobasic to dibasic form of the salt, (i.e., of potassium or sodium). In some aspects, the ratio of the monobasic and dibasic forms of sodium phosphate and potassium phosphate can be used to adjust the pH of the composition until the pH of the composition is between 7.0 and 7.4, e.g., 7.1 and 7.3, e.g., 7.2. In some aspects, the upper limit of pH is due to the destruction of the lipids of the disclosed EVs, e.g., exosomes, which undergo hydrolysis more commonly known as saponification. In some aspects, the potassium salts stabilize the pH upon freezing.

In some aspects, the saccharide for the pharmaceutical composition at about pH 7.2 comprises a monosaccharide. In some aspects, the saccharide comprises a disaccharide. In some aspects, the saccharide comprises a trisaccharide. In some aspects, the saccharide comprises an oligosaccharide. In some aspects, the saccharide comprises a polysaccharide. In some aspects, the saccharide comprises a sugar alcohol. In some aspects, the saccharide comprises any combination of saccharides described herein.

In some aspects, the saccharide comprises lactose. In some aspects, the saccharide comprises glucose. In some aspects, the saccharide comprises sucrose. In some aspects, the saccharide comprises trehalose. In some aspects, the saccharide comprises dextrose. In some aspects, the saccharide comprises any combination of saccharides described herein.

In some aspects, the saccharide is a sugar alcohol. In some aspects, the saccharide is a sugar alcohol having a molecular weight of from about 90.00 g/mol to about 190.00 g/mol. In some aspects, the saccharide has a molecular weight of from about 180.00 g/mol to about 380.00 g/mol,

In some aspects, the sugar alcohol comprises glycerol. In some aspects, the sugar alcohol comprises sorbitol. In some aspects, the sugar alcohol comprises mannitol. In some aspects, the sugar alcohol comprises xylitol. In some aspects, the sugar alcohol comprises any combination of sugar alcohols described herein.

In some aspects, the saccharide is a sucrose or a trehalose. In some aspects, the pharmaceutical composition comprises a sucrose. In other aspects, the pharmaceutical composition comprises a trehalose. In some aspects, the sucrose concentration is about 5% w/v.

II.C. Composition II (Sucrose)

The present disclosure is also directed to an appropriate concentration of sucrose or trehalose in a pharmaceutical composition comprising an extracellular vesicle. The suitable amount of sucrose or trehalose in the composition stabilizes the composition and/or reduces any aggregates. In some aspects, the pharmaceutical composition comprises (i) an extracellular vesicle and (ii) a saccharide, which is a sucrose or a trehalose at a concentration of about 5% w/v.

The saccharide disclosed herein, e.g., a sucrose or a trehalose, at the concentration of 5% w/v, can provide superior stability to a composition comprising 1% w/v sucrose. In particular, pharmaceutical compositions comprising (i) an extracellular vesicle, e.g., exosome and (ii) a saccharide, which is a sucrose or a trehalose at a concentration of about 5% w/v, provide advantages, including, but not limited to: (i) reduced aggregation of EVs, (ii) improved stability of EVs, (iii) improved integrity of the EV architecture, (iv) improved stability of engineered proteins contained on or in EVs, and (v) improved stability of passively loaded or conjugated materials such as small molecule drugs or proteins. In some aspects, the composition has reduced aggregation compared to a reference composition comprising a sucrose or a trehalose at a concentration of 1% w/v to 4% w/v, e.g., 1%. In some aspects, the composition has improved stability compared to a reference composition comprising a sucrose or a trehalose at a concentration of 1% w/v to 4% w/v, e.g., 1%. In some aspects, the composition has improved integrity of the EV architecture compared to a reference composition comprising a sucrose or a trehalose at a concentration of 1% w/v to 4% w/v, e.g., 1%.

In some aspects, the composition has improved stability of engineered proteins contained on or in EVs compared to a reference composition comprising a sucrose or a trehalose at a concentration of 1% w/v to 4% w/v, e.g., 1%.

In some aspects, the composition has improved stability of passively loaded or conjugated materials such as small molecule drugs or proteins, compared to a reference composition comprising a sucrose or a trehalose at a concentration of 1% w/v to 4% w/v, e.g., 1%.

In other aspects, the composition has improved stability compared to a reference composition comprising an sucrose or a trehalose at a concentration higher than 5.5% w/v, 6% w/v, 70% w/v, 80% w/v, 9% w/v, or 10% w/v.

The composition can further comprise sodium chloride, a potassium phosphate, a sodium chloride, or any combination thereof disclosed elsewhere herein.

II.D. Sodium Chloride

In some aspects, composition I or composition II further comprises sodium chloride.

In some aspects, the sodium chloride is present in the composition at a concentration of between about 10 mM and about 200 mM sodium chloride. In some aspects, the sodium chloride is present in the composition at a concentration of between about 10 mM and about 134 mM, between about 10 mM to about 130 mM, between about 20 mM to about 120 mM, between about 30 mM to about 110 mM, between about 40 mM to about 100 mM, between about 50 mM to about 90 mM, between about 60 mM to about 80 mM, between about 70 mM to about 80 mM, between about 45 mM to about 95 mM, between about 45 mM to about 80 mM, between about 45 mM to about 70 mM, between about 45 mM to about 65 mM, between about 50 mM to about 65 mM, between about 50 mM to about 60 mM, between about 50 mM to about 55 mM, between about 50 mM to about 55 mM, or between about 51 mM to about 54 mM. In some aspects, the concentration of sodium chloride is between about 10 mM to about 190 mM. In some aspects, the concentration of sodium chloride is between about 10 mM to about 180 mM. In some aspects, the concentration of sodium chloride is between about 10 mM to about 170 mM. In some aspects, the concentration of sodium chloride is between about 10 mM to about 160 mM. In some aspects, the concentration of sodium chloride is between about 10 mM to about 150 mM. In some aspects, the concentration of sodium chloride is between about 10 mM to about 140 mM. In some aspects, the concentration of sodium chloride is between about 10 mM to about 130 mM. In some aspects, the concentration of sodium chloride is between about 20 mM to about 120 mM. In some aspects, the concentration of sodium chloride is between about 30 mM to about 110 mM. In some aspects, the concentration of sodium chloride is between about 40 mM to about 100 mM. In some aspects, the concentration of sodium chloride is between about 50 mM to about 90 mM. In some aspects, the concentration of sodium chloride is between about 60 mM to about 80 mM. In some aspects, the concentration of sodium chloride is between about 70 mM to about 80 mM. In some aspects, the concentration of sodium chloride is between about 45 mM to about 95 mM. In some aspects, the concentration of sodium chloride is between about 45 mM to about 80 mM. In some aspects, the concentration of sodium chloride is between about 45 mM to about 70 mM. In some aspects, the concentration of sodium chloride is between about 45 mM to about 65 mM. In some aspects, the concentration of sodium chloride is between about 50 mM to about 65 mM. In some aspects, the concentration of sodium chloride is between about 50 mM to about 60 mM. In some aspects, the concentration of sodium chloride is between about 50 mM to about 55 mM. In some aspects, the concentration of sodium chloride is between about 50 mM to about 55 mM. In some aspects, the concentration of sodium chloride is between about 51 mM to about 54 mM. In some aspects, the concentration of sodium chloride is between about 40 mM to about 60 mM. In some aspects, the concentration of sodium chloride is between about 45 mM to about 55 mM. In some aspects, the concentration of sodium chloride is between about 48 mM to about 53 mM. In some aspects, the concentration of sodium chloride is between about 40 mM to about 50 mM. In some aspects, the concentration of sodium chloride is between about 45 mM to about 50 mM.

In some aspects, the concentration of sodium chloride is about 10 mM, about 20 mM, about 30 mM, about 40 mM, about 50 mM, about 60 mM, about 70 mM, about 80 mM, about 90 mM, or about 100 mM. In some aspects, the concentration of sodium chloride is about 10 mM. In some aspects, the concentration of sodium chloride is about 20 mM. In some aspects, the concentration of sodium chloride is about 30 mM. In some aspects, the concentration of sodium chloride is about 40 mM. In some aspects, the concentration of sodium chloride is about 50 mM. In some aspects, the concentration of sodium chloride is about 60 mM. In some aspects, the concentration of sodium chloride is about 70 mM. In some aspects, the concentration of sodium chloride is about 80 mM. In some aspects, the concentration of sodium chloride is about 90 mM. In some aspects, the concentration of sodium chloride is about 100 mM.

In some aspects, the concentration of sodium chloride is about 110 mM, about 120 mM, about 130 mM, about 140 mM, about 150 mM, about 160 mM, about 170 mM, about 180 mM, about 190 mM, or about 200 mM. In some aspects, the concentration of sodium chloride is about 110 mM. In some aspects, the concentration of sodium chloride is about 120 mM. In some aspects, the concentration of sodium chloride is about 130 mM. In some aspects, the concentration of sodium chloride is about 140 mM. In some aspects, the concentration of sodium chloride is about 150 mM. In some aspects, the concentration of sodium chloride is about 160 mM. In some aspects, the concentration of sodium chloride is about 170 mM. In some aspects, the concentration of sodium chloride is about 180 mM. In some aspects, the concentration of sodium chloride is about 190 mM. In some aspects, the concentration of sodium chloride is about 200 mM.

In some aspects, the concentration of sodium chloride is about 39 mM, about 40 mM, about 41 mM, about 42 mM, about 43 mM, about 44 mM, about 45 mM, about 46 mM, about 47 mM, about 48 mM, about 49 mM, or about 50 mM. In some aspects, the concentration of sodium chloride is about 39 mM. In some aspects, the concentration of sodium chloride is about 40 mM. In some aspects, the concentration of sodium chloride is about 41 mM. In some aspects, the concentration of sodium chloride is about 42 mM. In some aspects, the concentration of sodium chloride is about 43 mM. In some aspects, the concentration of sodium chloride is about 44 mM. In some aspects, the concentration of sodium chloride is about 45 mM. In some aspects, the concentration of sodium chloride is about 46 mM. In some aspects, the concentration of sodium chloride is about 47 mM. In some aspects, the concentration of sodium chloride is about 48 mM. In some aspects, the concentration of sodium chloride is about 49 mM. In some aspects, the concentration of sodium chloride is about 50 mM. In some aspects, the concentration of sodium chloride is 40.0. In some aspects, the concentration of sodium chloride is 49.6 mM.

Any of the concentrations of sodium chloride disclosed herein can be expressed in terms of weight per volume, e.g., mg/ml. A person of ordinary skill would be able to readily convert the mM concentrations disclosed herein to weight per volume concentrations. In some aspects, the concentration of sodium chloride is at least about 0.5 mg/ml to at least about 12 mg/ml. In some aspects, the concentration of sodium chloride is at least about 0.5 mg/ml to at least about 11.9 mg/ml, at least about 0.5 mg/ml to at least about 11.8 mg/ml, at least about 0.5 mg/ml to at least about 11.7 mg/ml, at least about 0.5 mg/ml to at least about 11.5 mg/ml, at least about 0.5 mg/ml to at least about 11.5 mg/ml, at least about 0.5 mg/ml to at least about 11.0 mg/ml, at least about 0.5 mg/ml to at least about 10.5 mg/ml, at least about 0.5 mg/ml to at least about 10 mg/ml, at least about 0.5 mg/ml to at least about 9.5 mg/ml, at least about 0.5 mg/ml to at least about 9 mg/ml, at least about 0.5 mg/ml to at least about 8.5 mg/ml, at least about 0.5 mg/ml to at least about 8.0 mg/ml, at least about 0.5 mg/ml to at least about 7.5 mg/ml, at least about 0.5 mg/ml to at least about 7.0 mg/ml, at least about 0.5 mg/ml to at least about 6.5 mg/ml, at least about 0.5 mg/ml to at least about 6.0 mg/ml, at least about 0.5 mg/ml to at least about 5.5 mg/ml, at least about 0.5 mg/ml to at least about 5.0 mg/ml, at least about 0.5 mg/ml to at least about 4.5 mg/ml, at least about 0.5 mg/ml to at least about 4.0 mg/ml, at least about 0.5 mg/ml to at least about 3.9 mg/ml, at least about 0.5 mg/ml to at least about 3.8 mg/ml, at least about 0.5 mg/ml to at least about 3.7 mg/ml, at least about 0.5 mg/ml to at least about 3.6 mg/ml, at least about 0.5 mg/ml to at least about 3.5 mg/ml, at least about 0.5 mg/ml to at least about 3.4 mg/ml, at least about 0.5 mg/ml to at least about 3.3 mg/ml, at least about 0.5 mg/ml to at least about 3.1 mg/ml, at least about 0.5 mg/ml to at least about 3.0 mg/ml, at least about 0.5 mg/ml to at least about 2.9 mg/ml, at least about 0.5 mg/ml to at least about 2.8 mg/ml, at least about 0.5 mg/ml to at least about 2.7 mg/ml, at least about 0.5 mg/ml to at least about 2.6 mg/ml, at least about 0.5 mg/ml to at least about 2.5 mg/ml, at least about 0.5 mg/ml to at least about 2.4 mg/ml, at least about 0.5 mg/ml to at least about 2.3 mg/ml, or at least about 0.5 mg/ml to at least about 2.1 mg/ml.

In some aspects, the concentration of sodium chloride is at least about 3.0 mg/ml. In some aspects, the concentration of sodium chloride is at least about 2.92 mg/ml. In some aspects, the concentration of sodium chloride is at least about 2.9 mg/ml. In some aspects, the concentration of sodium chloride is at least about 2.8 mg/ml. In some aspects, the concentration of sodium chloride is at least about 2.7 mg/ml. In some aspects, the concentration of sodium chloride is at least about 2.6 mg/ml. In some aspects, the concentration of sodium chloride is at least about 2.5 mg/ml. In some aspects, the concentration of sodium chloride is at least about 2.4 mg/ml. In some aspects, the concentration of sodium chloride is at least about 2.34 mg/ml. In some aspects, the concentration of sodium chloride is at least about 2.3 mg/ml. In some aspects, the concentration of sodium chloride is at least about 2.2 mg/ml. In some aspects, the concentration of sodium chloride is at least about 2.1 mg/ml. In some aspects, the concentration of sodium chloride is at least about 2.0 mg/ml.

In some aspects, Composition II further comprises a phosphate buffer comprising a phosphate compound. Non-limiting examples of phosphate compounds include potassium phosphate, sodium phosphate, disodium hydrogen phosphate, potassium dihydrogen phosphate, dipotassium phosphate, and/or a combination thereof. In some aspects, the phosphate compound is a potassium phosphate. In other aspects, the phosphate compound is a sodium phosphate.

II.E. Phosphates

In some aspects, Composition I or Composition II further comprises a potassium phosphate, e.g., potassium phosphate monobasic. In some aspects, a potassium phosphate, e.g., potassium phosphate monobasic, is present in the composition at a concentration of between about 1 mM to about 20 mM, about 2 mM to about 20 mM, about 3 mM to about 20 mM, about 4 mM to about 20 mM, about 5 mM to about 20 mM, about 6 mM to about 20 mM, about 7 mM to about 20 mM, about 8 mM to about 20 mM, about 1 mM to about 20 mM, about 2 mM to about 19 mM, about 3 mM to about 18 mM, about 4 mM to about 17 mM, about 5 mM to about 16 mM, or about 5 mM to about 15 mM.

In some aspects, the concentration of the potassium phosphate, e.g., potassium phosphate monobasic, is about 4.5 mM, about 4.6 mM, about 4.7 mM, about 4.8 mM, about 4.9 mM, about 5.0 mM, about 5.1 mM, about 5.2 mM, about 5.3 mM, about 5.4 mM, or about 5.5 mM. In some aspects, the potassium phosphate is present in the composition at a concentration of between about 2 mM to about 19 mM. In some aspects, the potassium phosphate is present in the composition at a concentration of between about 3 mM to about 18 mM. In some aspects, the potassium phosphate is present in the composition at a concentration of between about 4 mM to about 17 mM. In some aspects, the potassium phosphate is present in the composition at a concentration of between about 5 mM to about 16 mM. In some aspects, the potassium phosphate is present in the composition at a concentration of between about 5 mM to about 15 mM. In some aspects, the potassium phosphate is present in the composition at a concentration of between about 1 mM to about 10 mM. In some aspects, the potassium phosphate is present in the composition at a concentration of between about 2 mM to about 9 mM. In some aspects, the potassium phosphate is present in the composition at a concentration of between about 3 mM to about 8 mM. In some aspects, the potassium phosphate is present in the composition at a concentration of between about 4 mM to about 7 mM. In some aspects, the potassium phosphate is present in the composition at a concentration of between about 4 mM to about 6 mM. In some aspects, the potassium phosphate is present in the composition at a concentration of between about 3 mM to about 7 mM. In some aspects, the potassium phosphate is present in the composition at a concentration of between about 3 mM to about 6 mM.

In some aspects, the potassium phosphate, e.g., potassium phosphate monobasic, is present in the composition at a concentration of about 3.0 mM. In some aspects, the potassium phosphate, e.g., potassium phosphate monobasic, is present in the composition at a concentration of about 3.5 mM. In some aspects, the potassium phosphate, e.g., potassium phosphate monobasic, is present in the composition at a concentration of about 4.0 mM. In some aspects, the potassium phosphate, e.g., potassium phosphate monobasic, is present in the composition at a concentration of about 4.1 mM. In some aspects, the potassium phosphate, e.g., potassium phosphate monobasic, is present in the composition at a concentration of about 4.2 mM. In some aspects, the potassium phosphate, e.g., potassium phosphate monobasic, is present in the composition at a concentration of about 4.3 mM. In some aspects, the potassium phosphate, e.g., potassium phosphate monobasic, is present in the composition at a concentration of about 4.4 mM. In some aspects, the potassium phosphate, e.g., potassium phosphate monobasic, is present in the composition at a concentration of about 4.5 mM. In some aspects, the potassium phosphate is present in the composition at a concentration of about 4.6 mM. In some aspects, the potassium phosphate is present in the composition at a concentration of about 4.7 mM. In some aspects, the potassium phosphate is present in the composition at a concentration of about 4.8 mM. In some aspects, the potassium phosphate is present in the composition at a concentration of about 4.9 mM. In some aspects, the potassium phosphate is present in the composition at a concentration of about 5.0 mM. In some aspects, the potassium phosphate is present in the composition at a concentration of about 5.1 mM. In some aspects, the potassium phosphate is present in the composition at a concentration of about 5.2 mM. In some aspects, the potassium phosphate is present in the composition at a concentration of about 5.3 mM. In some aspects, the potassium phosphate is present in the composition at a concentration of about 5.4 mM. In some aspects, the potassium phosphate is present in the composition at a concentration of about 5.5 mM. In some aspects, the potassium phosphate is present in the composition at a concentration of about 5.6 mM. In some aspects, the potassium phosphate is present in the composition at a concentration of about 5.7 mM. In some aspects, the potassium phosphate is present in the composition at a concentration of about 5.8 mM. In some aspects, the potassium phosphate is present in the composition at a concentration of about 5.9 mM. In some aspects, the potassium phosphate is present in the composition at a concentration of about 6.0 mM. In some aspects, the concentration of the potassium phosphate in the composition is 5.15 mM.

In some aspects, the concentration of the potassium phosphate, e.g., potassium phosphate monobasic, is about 15.0 mM. In some aspects, the concentration of the potassium phosphate is about 15.1 mM. In some aspects, the concentration of the potassium phosphate is about 15.2 mM. In some aspects, the concentration of the potassium phosphate is about 15.3 mM. In some aspects, the concentration of the potassium phosphate is about 15.4 mM. In some aspects, the concentration of the potassium phosphate is about 15.5 mM. In some aspects, the concentration of the potassium phosphate is about 15.6 mM. In some aspects, the concentration of the potassium phosphate is about 15.7 mM. In some aspects, the concentration of the potassium phosphate is about 15.8 mM. In some aspects, the concentration of the potassium phosphate is about 15.9 mM. In some aspects, the concentration of the potassium phosphate is about 16.0. In some aspects, the concentration of the potassium phosphate is about 16.1 mM. In some aspects, the concentration of the potassium phosphate is about 16.2 mM. In some aspects, the concentration of the potassium phosphate is about 16.3 mM. In some aspects, the concentration of the potassium phosphate is about 16.4 mM. In some aspects, the concentration of the potassium phosphate is about 16.5 mM. In some aspects, the concentration of the potassium phosphate in the composition is 15.4 mM.

In some aspects, the concentration of the potassium phosphate, e.g., potassium phosphate monobasic, is about 14.0 mM. In some aspects, the concentration of the potassium phosphate, e.g., potassium phosphate monobasic, is about 14.1 mM. In some aspects, the concentration of the potassium phosphate, e.g., potassium phosphate monobasic, is about 14.2 mM. In some aspects, the concentration of the potassium phosphate, e.g., potassium phosphate monobasic, is about 14.3 mM. In some aspects, the concentration of the potassium phosphate, e.g., potassium phosphate monobasic, is about 14.4 mM. In some aspects, the concentration of the potassium phosphate, e.g., potassium phosphate monobasic, is about 14.5 mM. In some aspects, the concentration of the potassium phosphate, e.g., potassium phosphate monobasic, is about 14.6 mM. In some aspects, the concentration of the potassium phosphate, e.g., potassium phosphate monobasic, is about 14.7 mM. In some aspects, the concentration of the potassium phosphate, e.g., potassium phosphate monobasic, is about 14.8 mM. In some aspects, the concentration of the potassium phosphate, e.g., potassium phosphate monobasic, is about 14.9 mM.

Any of the concentrations of potassium phosphate monobasic disclosed herein can be expressed in terms of weight per volume, e.g., mg/ml. A person of ordinary skill would be able to readily convert the mM concentrations disclosed herein to weight per volume concentrations. In some aspects, the concentration of the potassium phosphate, e.g., potassium phosphate monobasic, is from at least about 0.14 mg/ml to at least about 2.75 mg/ml. In some aspects, the concentration of the potassium phosphate, e.g., potassium phosphate monobasic, is at least about 0.14 mg/ml. In some aspects, the concentration of the potassium phosphate, e.g., potassium phosphate monobasic, is at least about 0.15 mg/ml. In some aspects, the concentration of the potassium phosphate, e.g., potassium phosphate monobasic, is at least about 0.17 mg/ml. In some aspects, the concentration of the potassium phosphate, e.g., potassium phosphate monobasic, is at least about 0.2 mg/ml. In some aspects, the concentration of the potassium phosphate, e.g., potassium phosphate monobasic, is at least about 0.23 mg/ml. In some aspects, the concentration of the potassium phosphate, e.g., potassium phosphate monobasic, is at least about 0.25 mg/ml. In some aspects, the concentration of the potassium phosphate, e.g., potassium phosphate monobasic, is at least about 0.5 mg/ml. In some aspects, the concentration of the potassium phosphate, e.g., potassium phosphate monobasic, is at least about 0.60 mg/ml. In some aspects, the concentration of the potassium phosphate, e.g., potassium phosphate monobasic, is at least about 0.61 mg/ml. In some aspects, the concentration of the potassium phosphate, e.g., potassium phosphate monobasic, is at least about 0.62 mg/ml. In some aspects, the concentration of the potassium phosphate, e.g., potassium phosphate monobasic, is at least about 0.63 mg/ml. In some aspects, the concentration of the potassium phosphate, e.g., potassium phosphate monobasic, is at least about 0.64 mg/ml. In some aspects, the concentration of the potassium phosphate, e.g., potassium phosphate monobasic, is at least about 0.65 mg/ml. In some aspects, the concentration of the potassium phosphate, e.g., potassium phosphate monobasic, is at least about 0.66 mg/ml. In some aspects, the concentration of the potassium phosphate, e.g., potassium phosphate monobasic, is at least about 0.67 mg/ml. In some aspects, the concentration of the potassium phosphate, e.g., potassium phosphate monobasic, is at least about 0.68 mg/ml. In some aspects, the concentration of the potassium phosphate, e.g., potassium phosphate monobasic, is at least about 0.69 mg/ml. In some aspects, the concentration of the potassium phosphate, e.g., potassium phosphate monobasic, is at least about 0.70 mg/ml. In some aspects, the concentration of the potassium phosphate, e.g., potassium phosphate monobasic, is at least about 0.71 mg/ml. In some aspects, the concentration of the potassium phosphate, e.g., potassium phosphate monobasic, is at least about 0.72 mg/ml. In some aspects, the concentration of the potassium phosphate, e.g., potassium phosphate monobasic, is at least about 0.73 mg/ml. In some aspects, the concentration of the potassium phosphate, e.g., potassium phosphate monobasic, is at least about 0.74 mg/ml. In some aspects, the concentration of the potassium phosphate, e.g., potassium phosphate monobasic, is at least about 0.75 mg/ml. In some aspects, the concentration of the potassium phosphate, e.g., potassium phosphate monobasic, is at least about 1.0 mg/ml. In some aspects, the concentration of the potassium phosphate, e.g., potassium phosphate monobasic, is at least about 1.25 mg/ml. In some aspects, the concentration of the potassium phosphate, e.g., potassium phosphate monobasic, is at least about 1.50 mg/ml. In some aspects, the concentration of the potassium phosphate, e.g., potassium phosphate monobasic, is at least about 1.75 mg/ml. In some aspects, the concentration of the potassium phosphate, e.g., potassium phosphate monobasic, is at least about 2.0 mg/ml. In some aspects, the concentration of the potassium phosphate, e.g., potassium phosphate monobasic, is at least about 2.03 mg/ml. In some aspects, the concentration of the potassium phosphate, e.g., potassium phosphate monobasic, is at least about 2.04 mg/ml. In some aspects, the concentration of the potassium phosphate, e.g., potassium phosphate monobasic, is at least about 2.05 mg/ml. In some aspects, the concentration of the potassium phosphate, e.g., potassium phosphate monobasic, is at least about 2.1 mg/ml. In some aspects, the concentration of the potassium phosphate, e.g., potassium phosphate monobasic, is at least about 2.2 mg/ml. In some aspects, the concentration of the potassium phosphate, e.g., potassium phosphate monobasic, is at least about 2.3 mg/ml. In some aspects, the concentration of the potassium phosphate, e.g., potassium phosphate monobasic, is at least about 2.4 mg/ml. In some aspects, the concentration of the potassium phosphate, e.g., potassium phosphate monobasic, is at least about 2.5 mg/ml. In some aspects, the concentration of the potassium phosphate, e.g., potassium phosphate monobasic, is at least about 2.6 mg/ml. In some aspects, the concentration of the potassium phosphate, e.g., potassium phosphate monobasic, is at least about 2.7 mg/ml. In some aspects, the concentration of the potassium phosphate, e.g., potassium phosphate monobasic, is at least about 2.8 mg/ml. In some aspects, the concentration of the potassium phosphate, e.g., potassium phosphate monobasic, is at least about 2.9 mg/ml. In some aspects, the concentration of the potassium phosphate, e.g., potassium phosphate monobasic, is at least about 3.0 mg/ml.

In some aspects, Composition I or Composition II further comprises a sodium phosphate. In some aspects, the sodium phosphate, e.g., sodium phosphate dibasic heptahydrate, is present in the composition at a concentration of between about 10 mM to about 100 mM, between about 10 mM to about 90 mM, between about 10 mM to about 80 mM, between about 10 mM to about 70 mM, between about 10 mM to about 60 mM, between about 10 mM to about 50 mM, between about 10 mM to about 40 mM, between about 11 mM to about 29 mM, between about 12 mM to about 28 mM, between about 13 mM to about 27 mM, or between about 14 mM to about 26 mM. In some aspects, the sodium phosphate is present in the composition at a concentration of between about 11 mM to about 29 mM. In some aspects, the sodium phosphate is present in the composition at a concentration of between about 12 mM to about 28 mM. In some aspects, the sodium phosphate is present in the composition at a concentration of between about 13 mM to about 27 mM. In some aspects, the sodium phosphate is present in the composition at a concentration of between about 14 mM to about 26 mM.

In some aspects, the sodium phosphate is present in the composition at a concentration of about 14.5 mM, about 14.6 mM, about 14.7 mM, about 14.8 mM, about 14.9 mM, about 15.0 mM, about 15.1 mM about 15.2 mM, about 15.3 mM, about 15.4, mM, or about 15.5 mM. In some aspects, the sodium phosphate is present in the composition at a concentration of about 14.5 mM. In some aspects, the sodium phosphate is present in the composition at a concentration of about 14.6 mM. In some aspects, the sodium phosphate is present in the composition at a concentration of about 14.7 mM. In some aspects, the sodium phosphate is present in the composition at a concentration of about 14.8 mM. In some aspects, the sodium phosphate is present in the composition at a concentration of about 14.9 mM. In some aspects, the sodium phosphate is present in the composition at a concentration of about 15.0 mM. In some aspects, the sodium phosphate is present in the composition at a concentration of about 15.1 mM. In some aspects, the sodium phosphate is present in the composition at a concentration of about 15.2 mM. In some aspects, the sodium phosphate is present in the composition at a concentration of, about 15.3 mM. In some aspects, the sodium phosphate is present in the composition at a concentration of about 15.4 mM. In some aspects, the sodium phosphate is present in the composition at a concentration of about 15.5 mM. In some aspects, the concentration of the sodium phosphate is 14.9 mM.

In some aspects, the sodium phosphate is present in the composition at a concentration of about 15.6 mM. In some aspects, the sodium phosphate is present in the composition at a concentration of about 15.7 mM. In some aspects, the sodium phosphate is present in the composition at a concentration of about 15.8 mM. In some aspects, the sodium phosphate is present in the composition at a concentration of about 15.9 mM. In some aspects, the sodium phosphate is present in the composition at a concentration of about 16.0 mM. In some aspects, the sodium phosphate is present in the composition at a concentration of about 16.1 mM. In some aspects, the sodium phosphate is present in the composition at a concentration of about 16.2 mM. In some aspects, the sodium phosphate is present in the composition at a concentration of about 16.3 mM. In some aspects, the sodium phosphate is present in the composition at a concentration of about 16.4 mM. In some aspects, the sodium phosphate is present in the composition at a concentration of about 16.5 mM. In some aspects, the sodium phosphate is present in the composition at a concentration of about 16.6 mM. In some aspects, the sodium phosphate is present in the composition at a concentration of about 16.7 mM. In some aspects, the sodium phosphate is present in the composition at a concentration of about 16.8 mM. In some aspects, the sodium phosphate is present in the composition at a concentration of about 16.9 mM. In some aspects, the sodium phosphate is present in the composition at a concentration of about 17.0 mM.

In some aspects, the sodium phosphate is present in the composition at a concentration of about 26.5 mM. In some aspects, the sodium phosphate is present in the composition at a concentration of about 26.6 mM. In some aspects, the sodium phosphate is present in the composition at a concentration of about 26.7 mM. In some aspects, the sodium phosphate is present in the composition at a concentration of about 26.8 mM. In some aspects, the sodium phosphate is present in the composition at a concentration of about 26.9 mM. In some aspects, the sodium phosphate is present in the composition at a concentration of about 27.0 mM. In some aspects, the sodium phosphate is present in the composition at a concentration of about 27.1 mM. In some aspects, the sodium phosphate is present in the composition at a concentration of about 27.2, mM. In some aspects, the sodium phosphate is present in the composition at a concentration of about 27.3 mM. In some aspects, the sodium phosphate is present in the composition at a concentration of about 27.4 mM. In some aspects, the sodium phosphate is present in the composition at a concentration of about 27.5 mM. In some aspects, the sodium phosphate is present in the composition at a concentration of about 27.6 mM. In some aspects, the sodium phosphate is present in the composition at a concentration of about 27.7 mM. In some aspects, the sodium phosphate is present in the composition at a concentration of about 27.8 mM. In some aspects, the sodium phosphate is present in the composition at a concentration of about 27.9 mM. In some aspects, the sodium phosphate is present in the composition at a concentration of about 28.0 mM. In some aspects, the sodium phosphate is present in the composition at a concentration of the concentration of the sodium phosphate is 27.1 mM.

Any of the concentrations of sodium phosphate monobasic herein can be expressed in terms of weight per volume, e.g., mg/ml. A person of ordinary skill would be able to readily convert the mM concentrations disclosed herein to weight per volume concentrations. In some aspects, the sodium phosphate is present in the composition at a concentration from at least about 1.42 mg/ml to at least about 14.2 mg/ml. In some aspects, the sodium phosphate is present in the composition at a concentration of at least about 1.4 mg/ml. In some aspects, the sodium phosphate is present in the composition at a concentration of at least about 1.5 mg/ml. In some aspects, the sodium phosphate is present in the composition at a concentration of at least about 1.6 mg/ml. In some aspects, the sodium phosphate is present in the composition at a concentration of at least about 1.7 mg/ml. In some aspects, the sodium phosphate is present in the composition at a concentration of at least about 1.8 mg/ml. In some aspects, the sodium phosphate is present in the composition at a concentration of at least about 1.9 mg/ml. In some aspects, the sodium phosphate is present in the composition at a concentration of at least about 2.0 mg/ml. In some aspects, the sodium phosphate is present in the composition at a concentration of at least about 2.1 mg/ml. In some aspects, the sodium phosphate is present in the composition at a concentration of at least about 2.13 mg/ml. In some aspects, the sodium phosphate is present in the composition at a concentration of at least about 2.2 mg/ml. In some aspects, the sodium phosphate is present in the composition at a concentration of at least about 2.25 mg/ml. In some aspects, the sodium phosphate is present in the composition at a concentration of at least about 2.3 mg/ml. In some aspects, the sodium phosphate is present in the composition at a concentration of at least about 2.4 mg/ml. In some aspects, the sodium phosphate is present in the composition at a concentration of at least about 2.5 mg/ml. In some aspects, the sodium phosphate is present in the composition at a concentration of at least about 2.6 mg/ml. In some aspects, the sodium phosphate is present in the composition at a concentration of at least about 2.7 mg/ml. In some aspects, the sodium phosphate is present in the composition at a concentration of at least about 2.75 mg/ml. In some aspects, the sodium phosphate is present in the composition at a concentration of at least about 2.8 mg/ml. In some aspects, the sodium phosphate is present in the composition at a concentration of at least about 2.9 mg/ml. In some aspects, the sodium phosphate is present in the composition at a concentration of at least about 3.0 mg/ml. In some aspects, the sodium phosphate is present in the composition at a concentration of at least about 3.25 mg/ml. In some aspects, the sodium phosphate is present in the composition at a concentration of at least about 3.5 mg/ml. In some aspects, the sodium phosphate is present in the composition at a concentration of at least about 3.75 mg/ml. In some aspects, the sodium phosphate is present in the composition at a concentration of at least about 3.8 mg/ml. In some aspects, the sodium phosphate is present in the composition at a concentration of at least about 3.83 mg/ml. In some aspects, the sodium phosphate is present in the composition at a concentration of at least about 3.85 mg/ml. In some aspects, the sodium phosphate is present in the composition at a concentration of at least about 3.9 mg/ml. In some aspects, the sodium phosphate is present in the composition at a concentration of at least about 4.0 mg/ml. In some aspects, the sodium phosphate is present in the composition at a concentration of at least about 4.25 mg/ml. In some aspects, the sodium phosphate is present in the composition at a concentration of at least about 4.5 mg/ml. In some aspects, the sodium phosphate is present in the composition at a concentration of at least about 4.75 mg/ml. In some aspects, the sodium phosphate is present in the composition at a concentration of at least about 5.0 mg/ml. In some aspects, the sodium phosphate is present in the composition at a concentration of at least about 5.5 mg/ml. In some aspects, the sodium phosphate is present in the composition at a concentration of at least about 6.0 mg/ml. In some aspects, the sodium phosphate is present in the composition at a concentration of at least about 6.5 mg/ml. In some aspects, the sodium phosphate is present in the composition at a concentration of at least about 7.0 mg/ml. In some aspects, the sodium phosphate is present in the composition at a concentration of at least about 7.5 mg/ml. In some aspects, the sodium phosphate is present in the composition at a concentration of at least about 8.0 mg/ml. In some aspects, the sodium phosphate is present in the composition at a concentration of at least about 8.5 mg/ml. In some aspects, the sodium phosphate is present in the composition at a concentration of at least about 9.0 mg/ml. In some aspects, the sodium phosphate is present in the composition at a concentration of at least about 9.5 mg/ml. In some aspects, the sodium phosphate is present in the composition at a concentration of at least about 10.0 mg/ml. In some aspects, the sodium phosphate is present in the composition at a concentration of at least about 11.0 mg/ml. In some aspects, the sodium phosphate is present in the composition at a concentration of at least about 12.0 mg/ml. In some aspects, the sodium phosphate is present in the composition at a concentration of at least about 13.0 mg/ml. In some aspects, the sodium phosphate is present in the composition at a concentration of at least about 14.0 mg/ml. In some aspects, the sodium phosphate is present in the composition at a concentration of at least about 14.0 mg/ml.

In some aspects, sodium phosphate is sodium phosphate dibasic heptahydrate. In some aspects, the composition comprises sodium phosphate dibasic heptahydrate at a concentration of at least about 6.0 mg/ml. In some aspects, the composition comprises sodium phosphate dibasic heptahydrate at a concentration of at least about 6.1 mg/ml. In some aspects, the composition comprises sodium phosphate dibasic heptahydrate at a concentration of at least about 6.2 mg/ml. In some aspects, the composition comprises sodium phosphate dibasic heptahydrate at a concentration of at least about 6.3 mg/ml. In some aspects, the composition comprises sodium phosphate dibasic heptahydrate at a concentration of at least about 6.4 mg/ml. In some aspects, the composition comprises sodium phosphate dibasic heptahydrate at a concentration of at least about 6.5 mg/ml. In some aspects, the composition comprises sodium phosphate dibasic heptahydrate at a concentration of at least about 6.6 mg/ml. In some aspects, the composition comprises sodium phosphate dibasic heptahydrate at a concentration of at least about 6.7 mg/ml. In some aspects, the composition comprises sodium phosphate dibasic heptahydrate at a concentration of at least about 6.8 mg/ml. In some aspects, the composition comprises sodium phosphate dibasic heptahydrate at a concentration of at least about 6.9 mg/ml. In some aspects, the composition comprises sodium phosphate dibasic heptahydrate at a concentration of at least about 7.0 mg/ml. In some aspects, the composition comprises sodium phosphate dibasic heptahydrate at a concentration of at least about 7.1 mg/ml. In some aspects, the composition comprises sodium phosphate dibasic heptahydrate at a concentration of at least about 7.2 mg/ml. In some aspects, the composition comprises sodium phosphate dibasic heptahydrate at a concentration of at least about 7.24 mg/ml. In some aspects, the composition comprises sodium phosphate dibasic heptahydrate at a concentration of at least about 7.3 mg/ml. In some aspects, the composition comprises sodium phosphate dibasic heptahydrate at a concentration of at least about 7.4 mg/ml. In some aspects, the composition comprises sodium phosphate dibasic heptahydrate at a concentration of at least about 7.5 mg/ml. In some aspects, the composition comprises sodium phosphate dibasic heptahydrate at a concentration of at least about 7.6 mg/ml. In some aspects, the composition comprises sodium phosphate dibasic heptahydrate at a concentration of at least about 7.7 mg/ml. In some aspects, the composition comprises sodium phosphate dibasic heptahydrate at a concentration of at least about 7.8 mg/ml. In some aspects, the composition comprises sodium phosphate dibasic heptahydrate at a concentration of at least about 7.9 mg/ml. In some aspects, the composition comprises sodium phosphate dibasic heptahydrate at a concentration of at least about 8.0 mg/ml. In some aspects, the composition comprises sodium phosphate dibasic heptahydrate at a concentration of at least about 8.5 mg/ml. In some aspects, the composition comprises sodium phosphate dibasic heptahydrate at a concentration of at least about 9.0 mg/ml. In some aspects, the composition comprises sodium phosphate dibasic heptahydrate at a concentration of at least about 9.5 mg/ml. In some aspects, the composition comprises sodium phosphate dibasic heptahydrate at a concentration of at least about 10.0 mg/ml. In some aspects, the composition comprises sodium phosphate dibasic heptahydrate at a concentration of at least about 10.5 mg/ml. In some aspects, the composition comprises sodium phosphate dibasic heptahydrate at a concentration of at least about 11.0 mg/ml. In some aspects, the composition comprises sodium phosphate dibasic heptahydrate at a concentration of at least about 11.5 mg/ml. In some aspects, the composition comprises sodium phosphate dibasic heptahydrate at a concentration of at least about 12.0 mg/ml. In some aspects, the composition comprises sodium phosphate dibasic heptahydrate at a concentration of at least about 12.5 mg/ml. In some aspects, the composition comprises sodium phosphate dibasic heptahydrate at a concentration of at least about 13.0 mg/ml. In some aspects, the composition comprises sodium phosphate dibasic heptahydrate at a concentration of at least about 13.5 mg/ml. In some aspects, the composition comprises sodium phosphate dibasic heptahydrate at a concentration of at least about 14.0 mg/ml. In some aspects, the composition comprises sodium phosphate dibasic heptahydrate at a concentration of at least about 14.1 mg/ml. In some aspects, the composition comprises sodium phosphate dibasic heptahydrate at a concentration of at least about 14.2 mg/ml. In some aspects, the composition comprises sodium phosphate dibasic heptahydrate at a concentration of at least about 14.3 mg/ml. In some aspects, the composition comprises sodium phosphate dibasic heptahydrate at a concentration of at least about 14.4 mg/ml. In some aspects, the composition comprises sodium phosphate dibasic heptahydrate at a concentration of at least about 14.5 mg/ml. In some aspects, the composition comprises sodium phosphate dibasic heptahydrate at a concentration of at least about 15.0 mg/ml.

In some aspects, the phosphate buffer comprises a potassium phosphate and a sodium phosphate at a ratio of about 1:about 2. In some aspects, the phosphate buffer comprises a potassium phosphate and a sodium phosphate at a ratio of about 1:about 3. In some aspects, the phosphate buffer comprises a potassium phosphate and a sodium phosphate at a ratio of about 1:about 4. In some aspects, the phosphate buffer comprises a potassium phosphate and a sodium phosphate at a ratio of about 1:about 5. In some aspects, the phosphate buffer comprises a potassium phosphate and a sodium phosphate at a ratio of about 1:about 3. In some aspects, the phosphate buffer comprises a potassium phosphate and a sodium phosphate at a ratio of about 1:about 2.

In some aspects, the present disclosure provides a pharmaceutical composition comprising (i) an extracellular vesicle, (ii) a potassium phosphate, and (iii) a sodium phosphate in a solution, wherein the molar ratio of the potassium phosphate and the sodium phosphate is about 1 to about 3 or about 1 to about 2.

II.F. Saccharides

Certain aspects of the present disclosure are directed to a pharmaceutical composition comprising an extracellular vesicle, a saccharide, sodium chloride, a potassium phosphate, and a sodium phosphate. As noted above, the saccharide can be a monosaccharide, a disaccharide, a trisaccharide, or any other saccharides. In some aspects, the saccharide is a sucrose. In some aspects, the saccharide is a trehalose.

In some aspects, the saccharide, e.g., sucrose or trehalose, is present in the composition at a concentration from at least about 1% to at least about 10%, from at least about 2% to at least about 9%, from at least about 3% to at least about 8%, from at least about 4% to at least about 7%, from at least about 4% to at least about 6%, from at least about 3% to at least about 7%, from at least about 5% to at least about 10%, from at least about 5% to at least about 9%, from at least about 5% to at least about 8%, or from at least about 5% to at least about 7%. In some aspects, the saccharide, e.g., sucrose or trehalose, is present in the composition at a concentration of at least about 1%. In some aspects, the saccharide, e.g., sucrose or trehalose, is present in the composition at a concentration of at least about 2%. In some aspects, the saccharide, e.g., sucrose or trehalose, is present in the composition at a concentration of at least about 3%. In some aspects, the saccharide, e.g., sucrose or trehalose, is present in the composition at a concentration of at least about 4%. In some aspects, the saccharide, e.g., sucrose or trehalose, is present in the composition at a concentration of at least about 5%. In some aspects, the saccharide, e.g., sucrose or trehalose, is present in the composition at a concentration of at least about 6%. In some aspects, the saccharide, e.g., sucrose or trehalose, is present in the composition at a concentration of at least about 7%. In some aspects, the saccharide, e.g., sucrose or trehalose, is present in the composition at a concentration of at least about 8%. In some aspects, the saccharide, e.g., sucrose or trehalose, is present in the composition at a concentration of at least about 9%. In some aspects, the saccharide, e.g., sucrose or trehalose, is present in the composition at a concentration of at least about 10%.

In some aspects, the composition comprises at least about 1% sucrose. In some aspects, the composition comprises at least about 2% sucrose. In some aspects, the composition comprises at least about 2.5% sucrose. In some aspects, the composition comprises at least about 3% sucrose. In some aspects, the composition comprises at least about 4% sucrose. In some aspects, the composition comprises at least about 5% sucrose. In some aspects, the composition comprises at least about 6% sucrose. In some aspects, the composition comprises at least about 7% sucrose. In some aspects, the composition comprises at least about 8% sucrose. In some aspects, the composition comprises at least about 9% sucrose. In some aspects, the composition comprises at least about 10% sucrose.

In some aspects, the composition comprises at least about 1% trehalose. In some aspects, the composition comprises at least about 2% trehalose. In some aspects, the composition comprises at least about 3% trehalose. In some aspects, the composition comprises at least about 4% trehalose. In some aspects, the composition comprises at least about 5% trehalose. In some aspects, the composition comprises at least about 6% trehalose. In some aspects, the composition comprises at least about 7% trehalose. In some aspects, the composition comprises at least about 8% trehalose. In some aspects, the composition comprises at least about 9% trehalose. In some aspects, the composition comprises at least about 10% trehalose.

In some aspects, the saccharide, e.g., sucrose or trehalose, is present in the composition at a concentration from at least about 10 mg/ml to at least about 100 mg/ml, from at least about 20 mg/ml to at least about 90 mg/ml, from at least about 30 mg/ml to at least about 80 mg/ml, from at least about 40 mg/ml to at least about 70 mg/ml, from at least about 40 mg/ml to at least about 60 mg/ml, from at least about 30 mg/ml to at least about 70 mg/ml, from at least about 50 mg/ml to at least about 100 mg/ml, from at least about 50 mg/ml to at least about 90 mg/ml, from at least about 50 mg/ml to at least about 80 mg/ml, or from at least about 50 mg/ml to at least about 70 mg/ml. In some aspects, the saccharide, e.g., sucrose or trehalose, is present in the composition at a concentration of at least about 10 mg/ml. In some aspects, the saccharide, e.g., sucrose or trehalose, is present in the composition at a concentration of at least about 20 mg/ml. In some aspects, the saccharide, e.g., sucrose or trehalose, is present in the composition at a concentration of at least about 30 mg/ml. In some aspects, the saccharide, e.g., sucrose or trehalose, is present in the composition at a concentration of at least about 40 mg/ml. In some aspects, the saccharide, e.g., sucrose or trehalose, is present in the composition at a concentration of at least about 50 mg/ml. In some aspects, the saccharide, e.g., sucrose or trehalose, is present in the composition at a concentration of at least about 60 mg/ml. In some aspects, the saccharide, e.g., sucrose or trehalose, is present in the composition at a concentration of at least about 70 mg/ml. In some aspects, the saccharide, e.g., sucrose or trehalose, is present in the composition at a concentration of at least about 80 mg/ml. In some aspects, the saccharide, e.g., sucrose or trehalose, is present in the composition at a concentration of at least about 90 mg/ml. In some aspects, the saccharide, e.g., sucrose or trehalose, is present in the composition at a concentration of at least about 100 mg/ml.

In some aspects, the composition comprises at least about 10 mg/ml sucrose. In some aspects, the composition comprises at least about 20 mg/ml sucrose. In some aspects, the composition comprises at least about 30 mg/ml sucrose. In some aspects, the composition comprises at least about 40 mg/ml sucrose. In some aspects, the composition comprises at least about 50 mg/ml sucrose. In some aspects, the composition comprises at least about 60 mg/ml sucrose. In some aspects, the composition comprises at least about 70 mg/ml sucrose. In some aspects, the composition comprises at least about 80 mg/ml sucrose. In some aspects, the composition comprises at least about 90 mg/ml sucrose. In some aspects, the composition comprises at least about 100 mg/ml sucrose.

In some aspects, the composition comprises at least about 10 mg/ml trehalose. In some aspects, the composition comprises at least about 20 mg/ml trehalose. In some aspects, the composition comprises at least about 30 mg/ml trehalose. In some aspects, the composition comprises at least about 40 mg/ml trehalose. In some aspects, the composition comprises at least about 50 mg/ml trehalose. In some aspects, the composition comprises at least about 60 mg/ml trehalose. In some aspects, the composition comprises at least about 70 mg/ml trehalose. In some aspects, the composition comprises at least about 80 mg/ml trehalose. In some aspects, the composition comprises at least about 90 mg/ml trehalose. In some aspects, the composition comprises at least about 100 mg/ml trehalose.

In certain aspects, the composition comprises at least about 2.5% sucrose, wherein the composition has improved stability compared to a similar composition comprising less than about 2% sucrose.

II.G. Conductivity

In some aspects, Composition I or Composition II of the present disclosure has a conductivity between about 6 mS/cm+/−10% and about 10 mS/cm+/−10%. In some aspects, the conductivity is between 6 mS/cm+/−10% and about 7 mS/cm+/−10%, between about 7 mS/cm+/−10% and about 8 mS/cm+/−10%, between about 8 mS/cm+/−10% and about 9 mS/cm+/−10%, or between about 9 mS/cm+/−10% and about 10 mS/cm+/−10%. In some aspects, the conductivity is about 6 mS/cm+/−10%, about 7 mS/cm+/−10%, about 8 mS/cm+/−10%, about 9 mS/cm+/−10%, or about 10 mS/cm+/−10%.

In some aspects, the composition has a conductivity between about 6 mS/cm+/−10% and about 10 mS/cm+/−10%. In some aspects, the conductivity is about 6 mS/cm+/−10%. In some aspects, the conductivity is about 7 mS/cm+/−10%. In some aspects, the conductivity is about 8 mS/cm+/−10%. In some aspects, the conductivity is about 9 mS/cm+/−10%. In some aspects, the conductivity is about 10 mS/cm+/−10%. In some aspects, the conductivity is 7.23 mS/cm+/−10%. In some aspects, the conductivity is 8.8 mS/cm+/−10%.

II.H. Anti-Oxidants

In some aspects, Composition I or Composition II of the present disclosure further comprises an anti-oxidant. In some aspects, the anti-oxidant comprises D-methionine, L-methionine. ascorbic acid, erythorbic acid, Na ascorbate, thioglycerol, cysteine, acetylcysteine, cystine, dithioerythreitol, glutathione, tocopherols, butylated hydroxyanisole (BHA), butylated hydroxytoluene (BHT), sodium bisulphate, sodium dithionite, A-Tocopherol, γ-Tocopherol, propyl gallate, ascorbyl palmitate, sodium metabisulfite, thiourea, sodium thiosulfate, propyl gallate, Vitamin C, N-acetyl cysteine, selenium, and sodium thioglycolate In some aspects, the anti-oxidant is methionine. In other aspects, the anti-oxidant is D-methionine. In other aspects, the anti-oxidant is L-methionine.

In some aspects, Composition I or II the antioxidant comprises a thiosulfate or a salt thereof. In some aspects, the thiosulphate or salt thereof comprises sodium thiosulphate.

In some aspects, a composition disclosed herein comprises an antireductant. In some aspects, the antireductant comprises EDTA, EGTA, CuSO4, S-adenosylmethionine, cysteine, or any combination thereof.

II.I. Protease Inhibitors

In some aspects, a composition disclosed herein comprises a protease inhibitor. Proteins such as thioredoxin can reduce proteins with disulfide bonds. Addition of inhibtors such as EDTA, EGTA, and CuSO4 can reduce the activity, especially of metalloproteases such as hexokinase. EEGTA/EDTA inhibit by chelating divalent cations. Accordingly, in some aspects, the composition further comprises a protease inhibitor selected from EDTA, EGTA, CuSO4, and any combination thereof. In some aspects, the temperature is reduced in order to reduce the activity of a protease.

II.J. Characteristics of Compositions

The compositions of the present disclosure have been formulated such that the EVs of the compositions are stable under fluctuating temperature conditions, e.g., when frozen and/or thawed and/or administrated to a subject. Without wishing to be bound, it is though that the combination of the presently disclosed saccharides, e.g., sucrose at about 5% w/v, with potassium phosphate and sodium phosphate, at the particular ratios presently disclosed, provides superior stability to the composition and the EVs contained therein. For example, in some aspects, the compositions of the present disclosure are capable of being stored for various lengths of time and at various temperatures, wherein the stability of the extracellular vesicle, e.g., exosome, is not reduced. Furthermore, in some aspects, the presently disclosed compositions can be formulated as a liquid in ambient temperature and then frozen by placing the compositions into a −80° C. freezer, and then thawed. Accordingly, in some aspects, the composition can be stored as a liquid, before freezing, the composition can be stored as a solid, while frozen, and the composition can be stored as a liquid, after thawing, without compromising the stability of the EV, as described below.

In some aspects, the composition can be stored as a liquid, before freezing. In some aspects, the composition can be stored as a liquid, before freezing, at temperatures between about 25° C. to about 1° C., wherein the stability of the EV, e.g., exosome, is not reduced. In some aspects, the composition can be stored as a liquid, before freezing, at about 25° C. to about 1° C., without compromising the stability of the EV, e.g., exosome.

In some aspects, the composition can be stored as a liquid, before freezing, for at least about 4 hours, at least about 10 hours, at least about 12 hours, at least about 15 hours, at least about 20 hours, at least about 24 hours. In some aspects, the composition is stored as a liquid, before freezing for about 4 hours to about 12 hours, about 5 hours to about 12 hours, about 6 hours to about 12 hours, about 4 hours to about 24 hours, about 6 hours to about 24 hours, about 12 hours to about 24 hours, or about 4 hours about 16 hours. In some aspects, the composition can be stored as a liquid, before freezing, for less than 36 hours, less than 30 days, less than 24 hours, less than 23 hours, less than 22 hours, less than 21 hours, less than 20 hours, less than 19 hours, less than 18 hours, less than 17 hours, less than 16 hours, less than 15 hours, less than 14 hours, less than 13 hours, less than 12 hours, less than 11 hours, less than 10 hours, less than 9 hours, less than 8 hours, less than 7 hours, less than 6 hours, less than 5 hours, or less than 4 hours.

In some aspects, the composition can be stored as a liquid at about 4° C. before freezing, for about one week. In some aspects, the composition can be stable for up to a week at 4° C. In some aspects, the composition can be stored as a liquid at about 4° C. before freezing, for about one week and then administered to a subject in need thereof.

In some aspects, the composition is capable of being stored as a frozen solid, for a length of time before being thawed. In some aspects, the composition can be stored as a solid, at zero and sub-zero temperatures, e.g., temperatures between about 0° C. and or −80° C., wherein the stability of the EV, e.g., exosome, is not reduced. In some aspects, the composition can be stored as a frozen solid, at temperatures between about 0° C. and or −80° C. In some aspects, the composition can be stored as a frozen solid, at temperatures between about 0° C. and −50° C. In some aspects, the composition can be stored as a frozen solid, at temperatures between about 0° C. and −20° C. In some aspects, the composition can be stored as a frozen solid, at temperatures between about 0° C. and −15° C. In some aspects, the composition can be stored for up to 6 months at −80° C. In some aspects, the composition can be stable for one year at −80° C. In some aspects, the composition can be stable for two years at −80° C.

The presently disclosed compositions can be stored as frozen solids for various lengths of time, and thereafter thawed, in preparation for administration to subjects in need thereof. In some aspects, the thawed liquids can be stored for various lengths and at various temperatures prior to administration, without compromising the stability of the EVs, e.g., exosomes. In some aspects, the composition is capable of being thawed and stored as a liquid, at a temperatures from about 1° C. to about 25° C., wherein the stability of the EV, e.g., exosome, is not reduced.

In some aspects, the composition can be stored as a thawed liquid, at about 1° C. In some aspects, the composition can be stored as a thawed liquid, at about 2° C. In some aspects, the composition can be stored as a thawed liquid, at about 3° C. In some aspects, the composition can be stored as a thawed liquid, at about 4° C. In some aspects, the composition can be stored as a thawed liquid, at about 5° C. In some aspects, the composition can be stored as a thawed liquid, at about 6° C. In some aspects, the composition can be stored as a thawed liquid, at about 7° C. In some aspects, the composition can be stored as a thawed liquid, at about 8° C. In some aspects, the composition can be stored as a thawed liquid, at about 9° C. In some aspects, the composition can be stored as a thawed liquid, at about 10° C. In some aspects, the composition can be stored as a thawed liquid, at about 11° C. In some aspects, the composition can be stored as a thawed liquid, at about 12° C. In some aspects, the composition can be stored as a thawed liquid, at about 13° C. In some aspects, the composition can be stored as a thawed liquid, at about 14° C. In some aspects, the composition can be stored as a thawed liquid, at about 15° C. In some aspects, the composition can be stored as a thawed liquid, at about 16° C. In some aspects, the composition can be stored as a thawed liquid, at about 17° C. In some aspects, the composition can be stored as a thawed liquid, at about 18° C. In some aspects, the composition can be stored as a thawed liquid, at about 19° C. In some aspects, the composition can be stored as a thawed liquid, at about 20° C. In some aspects, the composition can be stored as a thawed liquid, at about 21° C. In some aspects, the composition can be stored as a thawed liquid, at about 22° C. In some aspects, the composition can be stored as a thawed liquid, at about 23° C. In some aspects, the composition can be stored as a thawed liquid, at about 24° C. In some aspects, the composition can be stored as a thawed liquid, at about 25° C. In some aspects, the composition can be stored as a thawed liquid at 4° C., for about one week. In some aspects, the composition can be stable as a thawed liquid for up to a week at 4° C.

In some aspects, the composition can be stored, and then directly administered to a subject in need thereof. In some aspects, the composition can be stored for up to 24 hours at 25° C., and then directly administered to a subject in need thereof. In some aspects, the composition can be stored for up to 3 days at 4° C., and then directly administered to a subject in need thereof. In some aspects, the composition can be stored for up to 7 days at 4° C., and then directly administered to a subject in need thereof. In some aspects, the composition can be stored for up to 6 months at −80° C., thawed, and then directly administered to a subject in need thereof.

II.K. Exemplary Compositions

In some aspects, the composition of the present disclosure comprises:
(a) Extracellular vesicles;
(b) Sucrose at a concentration between about 4% w/v and about 6% w/v, e.g., 5% w/v;
(c) Sodium chloride at a concentration between 40 mM and about 60 mM;
(d) Potassium phosphate monobasic at a concentration between 4 mM and 6 mM; and
(e) sodium phosphate dibasic heptahydrate at a concentration between about 10 mM and about 20 mM, wherein the pH of the composition is about 7.2. In some aspects, the conductivity of the composition is about 7.2 mS/cm+/−10%. In some aspects, the composition is in solution, e.g., a liquid formulation.

In some aspects, the composition of the present disclosure comprises:
(a) Extracellular vesicles;
(b) Sucrose at a concentration of about 5% w/v;
(c) Sodium chloride at a concentration of about 50 mM;
(d) Potassium phosphate monobasic at a concentration of about 5 mM;
(e) sodium phosphate dibasic heptahydrate at a concentration of about 15 mM, wherein the pH of the composition is about 7.2. In some aspects, the conductivity of the composition is about 7.2 mS/cm+/−10%. In some aspects, the composition is in solution, e.g., a liquid formulation.

In some aspects, the composition of the present disclosure comprises:
(a) Extracellular vesicles;
(b) Sucrose at a concentration between about 4% w/v and about 6% w/v, e.g., 5% w/v;
(c) Sodium chloride at a concentration between 30 mM and about 50 mM;
(d) Potassium phosphate monobasic at a concentration between 10 mM and 20 mM;
(e) sodium phosphate dibasic heptahydrate at a concentration between about 20 mM and about 40 mM, wherein the pH of the composition is about 7.2. In some aspects, the conductivity of the composition is about 8.8 mS/cm+/−10%. In some aspects, the composition is in solution, e.g., a liquid formulation.

In some aspects, the composition of the present disclosure comprises:
(a) Extracellular vesicles;
(b) Sucrose at a concentration of about 5% w/v;
(c) Sodium chloride at a concentration of about 40 mM;
(d) Potassium phosphate monobasic at a concentration of about 15 mM;
(e) sodium phosphate dibasic heptahydrate at a concentration of about 27 mM, wherein the pH of the composition is about 7.2. In some aspects, the conductivity of the composition is about 8.8 mS/cm+/−10%. In some aspects, the composition is in solution, e.g., a liquid formulation.

In some aspects, the composition of the present disclosure comprises:
(a) Extracellular vesicles;
(b) Sucrose at a concentration between about 4% w/v and about 6% w/v, e.g., 5% w/v;
(c) Sodium chloride at a concentration between 30 mM and about 50 mM;
(d) Potassium phosphate monobasic at a concentration between 1 mM and 20 mM;
(e) sodium phosphate dibasic heptahydrate at a concentration between about 20 mM and about 40 mM, wherein the pH of the composition is about 7.2. In some aspects, the composition is in solution, e.g., a liquid formulation.

In some aspects, the composition of the present disclosure comprises:
(a) Extracellular vesicles;
(b) Sucrose at a concentration of about 5% w/v;
(c) Sodium chloride at a concentration of about 40 mM;
(d) Potassium phosphate monobasic at a concentration of about 11 mM;
(e) sodium phosphate dibasic heptahydrate at a concentration of about 32 mM, wherein the pH of the composition is about 7.2. In some aspects, the composition is in solution, e.g., a liquid formulation.

In some aspects, the composition of the present disclosure comprises:
(a) Extracellular vesicles;
(b) Sucrose at a concentration between about 4% w/v and about 6% w/v, e.g., 5% w/v;
(c) Sodium chloride at a concentration between 30 mM and about 60 mM;
(d) Potassium phosphate monobasic at a concentration between 1 mM and 20 mM;
(e) sodium phosphate dibasic heptahydrate at a concentration between about 10 mM and about 40 mM, wherein the pH of the composition is about 7.2. In some aspects, the composition is in solution, e.g., a liquid formulation.

In some aspects, the composition of the present disclosure comprises:
(a) Extracellular vesicles;
(b) Sucrose at a concentration between about 4% w/v and about 6% w/v, e.g., 5% w/v;
(c) Sodium chloride at a concentration between 40 mM and about 50 mM;
(d) Potassium phosphate monobasic at a concentration between 5 mM and 15 mM;
(e) sodium phosphate dibasic heptahydrate at a concentration between about 15 mM and about 35 mM, wherein the pH of the composition is about 7.2. In some aspects, the composition is in solution, e.g., a liquid formulation.

II.K.1. Exemplary Composition 02

In some aspects, the composition of the present disclosure comprises:
(a) Extracellular vesicles;
(b) Sucrose at a concentration between about 4% w/v and about 6% w/v, e.g., 5% w/v;
(c) Sodium chloride at a concentration between 30 mM and about 50 mM;
(d) Potassium phosphate monobasic at a concentration between about 5 mM and about 25 mM;
(e) Sodium phosphate dibasic heptahydrate at a concentration between about 15 mM and about 35 mM,
(f) wherein the pH of the composition is from about 6.7 to about 7.7.

In some aspects, the composition comprises:
(a) Extracellular vesicles;
(b) Sucrose at a concentration of about 5% w/v;
(c) Sodium chloride at a concentration between 30 mM and about 50 mM;
(d) Potassium phosphate monobasic at a concentration between about 5 mM and about 25 mM;
(e) Sodium phosphate dibasic heptahydrate at a concentration between about 15 mM and about 35 mM,
(f) wherein the pH of the composition is from about 6.7 to about 7.7.

In some aspects, the composition comprises:
(a) Extracellular vesicles;
(b) Sucrose at a concentration between about 4% w/v and about 6% w/v, e.g., 5% w/v;
(c) Sodium chloride at a concentration of about 40 mM;
(d) Potassium phosphate monobasic at a concentration between about 5 mM and about 25 mM;
(e) Sodium phosphate dibasic heptahydrate at a concentration between about 15 mM and about 35 mM,
(f) wherein the pH of the composition is from about 6.7 to about 7.7

In some aspects, the composition comprises:
(a) Extracellular vesicles;
(b) Sucrose at a concentration between about 4% w/v and about 6% w/v, e.g., 5% w/v;
(c) Sodium chloride at a concentration between about 30 mM and about 50 mM;
(d) Potassium phosphate monobasic at a concentration of about 15 mM;
(e) Sodium phosphate dibasic heptahydrate at a concentration between about 15 mM and about 35 mM,
(f) wherein the pH of the composition is from about 6.7 to about 7.7.

In some aspects, the composition comprises:
(a) Extracellular vesicles;
(b) Sucrose at a concentration between about 4% w/v and about 6% w/v, e.g., 5% w/v;
(c) Sodium chloride at a concentration between 30 mM and about 50 mM;
(d) Potassium phosphate monobasic at a concentration between about 5 mM and about 25 mM;
(e) Sodium phosphate dibasic heptahydrate at a concentration of about 30 mM,
(f) wherein the pH of the composition is from about 6.7 to about 7.7.

In some aspects, the composition comprises:
(a) Extracellular vesicles;
(b) Sucrose at a concentration between about 4% w/v and about 6% w/v, e.g., 5% w/v;
(c) Sodium chloride at a concentration between 30 mM and about 50 mM;
(d) Potassium phosphate monobasic at a concentration between about 5 mM and about 25 mM;
(e) Sodium phosphate dibasic heptahydrate at a concentration of about 27 mM,
(f) wherein the pH of the composition is from about 6.7 to about 7.7.

In some aspects, the composition comprises:
(a) Extracellular vesicles;
(b) Sucrose at a concentration between about 4% w/v and about 6% w/v, e.g., 5% w/v;
(c) Sodium chloride at a concentration between 30 mM and about 50 mM;
(d) Potassium phosphate monobasic at a concentration between about 5 mM and about 25 mM;
(e) Sodium phosphate dibasic heptahydrate at a concentration between about 15 mM and about 35 mM,
(f) wherein the pH of the composition is from about 7.0 to about 7.4.

In some aspects, the composition comprises:
(a) Extracellular vesicles;
(b) Sucrose at a concentration between about 4% w/v and about 6% w/v, e.g., 5% w/v;
(c) Sodium chloride at a concentration between 30 mM and about 50 mM;
(d) Potassium phosphate monobasic at a concentration between about 5 mM and about 25 mM;
(e) Sodium phosphate dibasic heptahydrate at a concentration between about 15 mM and about 35 mM,
(f) wherein the pH of the composition is about 7.2.

In certain aspects, the composition comprises:
(a) Extracellular vesicles;
(b) Sucrose at a concentration of about 5% w/v;
(c) Sodium chloride at a concentration of about 40 mM;
(d) Potassium phosphate monobasic at a concentration of about 15 mM;
(e) Sodium phosphate dibasic heptahydrate at a concentration of about 27 mM,
(f) wherein the pH of the composition is about 7.2.

In some aspects, the extracellular vesicles comprise a STING agonist. In some aspects, the STING agonist comprises a cyclic dinucleotide (CDN) STING agonist or a non-cyclic dinucleotide STING agonist. In some aspects, the STING agonist comprises cGMP, cyclic di-GMP (c-di-GMP), cAMP, cyclic di-AMP (c-di-AMP), cyclic-GMP-AMP (cGAMP), cyclic di-IMP (c-di-IMP), cyclic AMP-IMP (cAIMP), and any analogue thereof, are known to stimulate or enhance an immune or inflammation response in a patient. In some aspects, the CDN comprises 2′2′, 2′3′, 2′5′, 3′3′, or 3′5′ bonds linking the cyclic dinucleotides, or any combination thereof.

In some aspects, the composition is lyophilized.

II.K.1. Exemplary Composition 03

In certain aspects, the composition comprises:
(a) Extracellular vesicles;
(b) Sucrose at a concentration from at least about 50 mM to at least about 300 mM;
(c) Sodium chloride at a concentration from at least about 10 mM to at least about 200 mM;
(d) Potassium phosphate monobasic at a concentration from at least about 1 mM to at least about 20 mM;
(e) Sodium phosphate dibasic at a concentration from at least about 5 mM to at least about 35 mM,
(f) wherein the pH of the composition is from about 6.7 to about 7.7.

In certain aspects, the composition comprises:
(a) Extracellular vesicles;
(b) Sucrose at a concentration of about 146 mM;
(c) Sodium chloride at a concentration from at least about 10 mM to at least about 200 mM;
(d) Potassium phosphate monobasic at a concentration from at least about 1 mM to at least about 20 mM;
(e) Sodium phosphate dibasic at a concentration from at least about 5 mM to at least about 35 mM,
(f) wherein the pH of the composition is from about 6.7 to about 7.7.

In certain aspects, the composition comprises:
(a) Extracellular vesicles;
(b) Sucrose at a concentration from at least about 50 mM to at least about 300 mM;
(c) Sodium chloride at a concentration of about 50 mM;
(d) Potassium phosphate monobasic at a concentration from at least about 1 mM to at least about 20 mM;
(e) Sodium phosphate dibasic at a concentration from at least about 5 mM to at least about 35 mM,
(f) wherein the pH of the composition is from about 6.7 to about 7.7.

In certain aspects, the composition comprises:
(a) Extracellular vesicles;
(b) Sucrose at a concentration from at least about 50 mM to at least about 300 mM;
(c) Sodium chloride at a concentration from at least about 10 mM to at least about 200 mM;
(d) Potassium phosphate monobasic at a concentration of about 5 mM;
(e) Sodium phosphate dibasic at a concentration from at least about 5 mM to at least about 35 mM,
(f) wherein the pH of the composition is from about 6.7 to about 7.7.

In certain aspects, the composition comprises:
(a) Extracellular vesicles;
(b) Sucrose at a concentration from at least about 50 mM to at least about 300 mM;
(c) Sodium chloride at a concentration from at least about 10 mM to at least about 200 mM;
(d) Potassium phosphate monobasic at a concentration from at least about 1 mM to at least about 20 mM;
(e) Sodium phosphate dibasic at a concentration of about 15 mM,
(f) wherein the pH of the composition is from about 6.7 to about 7.7.

In certain aspects, the composition comprises:
(a) Extracellular vesicles;
(b) Sucrose at a concentration from at least about 50 mM to at least about 300 mM;
(c) Sodium chloride at a concentration from at least about 10 mM to at least about 200 mM;
(d) Potassium phosphate monobasic at a concentration from at least about 1 mM to at least about 20 mM;
(e) Sodium phosphate dibasic at a concentration from at least about 5 mM to at least about 35 mM,
(f) wherein the pH of the composition is about 7.2.

In certain aspects, the composition comprises:
(a) Extracellular vesicles;
(b) Sucrose at a concentration of about 146 mM;
(c) Sodium chloride at a concentration of about 50 mM;
(d) Potassium phosphate monobasic at a concentration of about 5 mM;
(e) Sodium phosphate dibasic at a concentration of about 15 mM,
(f) wherein the pH of the composition is about 7.2.

In certain aspects, the composition comprises:
(a) Extracellular vesicles;
(b) Sucrose at a concentration from at least about 1% to at least about 10%;
(c) Sodium chloride at a concentration from at least about 10 mM to at least about 200 mM;
(d) Potassium phosphate monobasic at a concentration from at least about 1 mM to at least about 20 mM;
(e) Sodium phosphate dibasic at a concentration from at least about 5 mM to at least about 35 mM,
(f) wherein the pH of the composition is from about 6.7 to about 7.7.

In certain aspects, the composition comprises:
(a) Extracellular vesicles;
(b) Sucrose at a concentration of about 5%;
(c) Sodium chloride at a concentration from at least about 10 mM to at least about 200 mM;
(d) Potassium phosphate monobasic at a concentration from at least about 1 mM to at least about 20 mM;
(e) Sodium phosphate dibasic at a concentration from at least about 5 mM to at least about 35 mM,
(f) wherein the pH of the composition is from about 6.7 to about 7.7.

In certain aspects, the composition comprises:
(a) Extracellular vesicles;
(b) Sucrose at a concentration from at least about 1% to at least about 10%;
(c) Sodium chloride at a concentration of about 50 mM;
(d) Potassium phosphate monobasic at a concentration from at least about 1 mM to at least about 20 mM;
(e) Sodium phosphate dibasic at a concentration from at least about 5 mM to at least about 35 mM,
(f) wherein the pH of the composition is from about 6.7 to about 7.7.

In certain aspects, the composition comprises:
(a) Extracellular vesicles;
(b) Sucrose at a concentration from at least about 1% to at least about 10%;
(c) Sodium chloride at a concentration from at least about 10 mM to at least about 200 mM;
(d) Potassium phosphate monobasic at a concentration of about 5 mM;
(e) Sodium phosphate dibasic at a concentration from at least about 5 mM to at least about 35 mM,
(f) wherein the pH of the composition is from about 6.7 to about 7.7.

In certain aspects, the composition comprises:
(a) Extracellular vesicles;
(b) Sucrose at a concentration from at least about 1% to at least about 10%;
(c) Sodium chloride at a concentration from at least about 10 mM to at least about 200 mM;
(d) Potassium phosphate monobasic at a concentration from at least about 1 mM to at least about 20 mM;
(e) Sodium phosphate dibasic at a concentration of about 15 mM,
(f) wherein the pH of the composition is from about 6.7 to about 7.7.

In certain aspects, the composition comprises:
(a) Extracellular vesicles;
(b) Sucrose at a concentration from at least about 1% to at least about 10%;
(c) Sodium chloride at a concentration from at least about 10 mM to at least about 200 mM;
(d) Potassium phosphate monobasic at a concentration from at least about 1 mM to at least about 20 mM;
(e) Sodium phosphate dibasic at a concentration from at least about 5 mM to at least about 35 mM,
(f) wherein the pH of the composition is about 7.2.

In certain aspects, the composition comprises:
(a) Extracellular vesicles;
(b) Sucrose at a concentration of about 5%;
(c) Sodium chloride at a concentration of about 50 mM;
(d) Potassium phosphate monobasic at a concentration of about 5 mM;
(e) Sodium phosphate dibasic at a concentration of about 15 mM,
(f) wherein the pH of the composition is about 7.2.

In some aspects, the extracellular vesicle comprises an IL-12 moiety. In certain aspects, the IL-12 moiety disclosed herein. In certain aspects, the IL-12 moiety comprises an amino acid sequence at least about 80%, at least about 85%, at least about 90%, at least about 95%, at least about 96%, at least about 97%, at least about 98%, or at least about 99% identical to the amino acid sequence set forth in SEQ ID NO: 11, 12, or 13 (Table 1A). In certain aspects, the IL-12 comprises the amino acid sequence set forth in SEQ ID NO: 11. In certain aspects, the IL-12 comprises the amino acid sequence set forth in SEQ ID NO: 12. In some aspects, the IL-12 comprises an amino acid sequence at least about 80%, at least about 85%, at least about 90%, at least about 95%, at least about 96%, at least about 97%, at least about 98%, or at least about 99% identical to the amino acid sequence set forth in SEQ ID NO: 13. In certain aspects, the IL-12 comprises the amino acid sequence set forth in SEQ ID NO: 13.

In certain aspects, the composition comprises:
(a) Extracellular vesicles comprising an IL-12 moiety, wherein the IL-12 moiety comprises an amino acid sequence at least about 80%, at least about 85%, at least about 90%, at least about 95%, at least about 96%, at least about 97%, at least about 98%, at least about 99%, or 100% identical to the amino acid sequence set forth in SEQ ID NO: 11, 12, or 13;
(b) Sucrose at a concentration of about 146 mM;
(c) Sodium chloride at a concentration of about 50 mM;
(d) Potassium phosphate monobasic at a concentration of about 5 mM;
(e) Sodium phosphate dibasic at a concentration of about 15 mM,
(f) wherein the pH of the composition is about 7.2.

In some aspects, the composition comprises:
(a) Extracellular vesicles comprising an IL-12 moiety, wherein the IL-12 moiety comprises an amino acid sequence at least about 80%, at least about 85%, at least about 90%, at least about 95%, at least about 96%, at least about 97%, at least about 98%, at least about 99%, or 100% identical to the amino acid sequence set forth in SEQ ID NO: 11, 12, or 13;
(b) Sucrose at a concentration of about 5%;
(c) Sodium chloride at a concentration of about 50 mM;
(d) Potassium phosphate monobasic at a concentration of about 5 mM;
(e) Sodium phosphate dibasic at a concentration of about 15 mM,
(f) wherein the pH of the composition is about 7.2.

In certain aspects, the composition comprises:
(a) Extracellular vesicles comprising an IL-12 moiety, wherein the IL-12 moiety comprises the amino acid set forth in SEQ ID NO: 13;
(b) Sucrose at a concentration of about 146 mM;
(c) Sodium chloride at a concentration of about 50 mM;
(d) Potassium phosphate monobasic at a concentration of about 5 mM;
(e) Sodium phosphate dibasic at a concentration of about 15 mM,
(f) wherein the pH of the composition is about 7.2.

In some aspects, the composition comprises:
(a) Extracellular vesicles comprising an IL-12 moiety, wherein the IL-12 moiety comprises the amino acid set forth in SEQ ID NO: 13;
(b) Sucrose at a concentration of about 5%;
(c) Sodium chloride at a concentration of about 50 mM;
(d) Potassium phosphate monobasic at a concentration of about 5 mM;
(e) Sodium phosphate dibasic at a concentration of about 15 mM,
(f) wherein the pH of the composition is about 7.2.

In some aspects, the extracellular vesicle comprises an ASO. In some aspects, the ASO comprises a contiguous nucleotide sequence of 10 to 30 nucleotides in length that is complementary to a nucleic acid sequence within a STAT6 transcript (SEQ ID NO: 23; Table 1). In some aspects, the ASO comprises a nucleic acid sequence selected from SEQ ID NOs: 91-193. In some aspects, the ASO comprises the nucleic acid sequence GAAAGGTTCCGTCGGGC (SEQ ID NO: 144). In some aspects, the ASO comprises the nucleic acid sequence CTGAGTCGCTGAAGCGG (SEQ ID NO: 145). In some aspects, the ASO comprises the nucleic acid sequence GCCCTTGTACTTTTGCATAG (SEQ ID NO: 193). In some aspects, the ASO comprises the nucleic acid sequence GCAAGATCCCGGATTCGGTC (SEQ ID NO: 185).

In certain aspects, the composition comprises:
(a) Extracellular vesicles comprising an ASO, wherein the ASO comprises a contiguous nucleotide sequence of 10 to 30 nucleotides in length that is complementary to a nucleic acid sequence within a STAT6 transcript;
(b) Sucrose;
(c) Sodium chloride;
(d) Potassium phosphate monobasic at a concentration of about 5 mM;
(e) Sodium phosphate dibasic at a concentration of about 15 mM,
(f) wherein the pH of the composition is about 7.2;
wherein the sucrose is at a concentration selected from about 73 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 146 mM, and about 150 mM; and
wherein the sodium chloride is at a concentration selected from about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 146 mM, and about 150 mM.

In certain aspects, the composition comprises:
(a) Extracellular vesicles comprising an ASO;
(b) Sucrose;
(c) Sodium chloride;
(d) Potassium phosphate monobasic at a concentration of about 5 mM;
(e) Sodium phosphate dibasic at a concentration of about 15 mM,
(f) wherein the pH of the composition is about 7.2;
wherein the sucrose is at a concentration selected from about 73 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 146 mM, and about 150 mM; and
wherein the sodium chloride is at a concentration selected from about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 146 mM, and about 150 mM. In some aspects, wherein the ASO comprises a nucleic acid sequence selected from SEQ ID NOs: 91-193.

In some aspects, the composition comprises:
(a) Extracellular vesicles comprising an ASO;
(b) Sucrose;
(c) Sodium chloride;
(d) Potassium phosphate monobasic at a concentration of about 5 mM;
(e) Sodium phosphate dibasic at a concentration of about 15 mM,
(f) wherein the pH of the composition is about 7.2;
wherein the sucrose is at a concentration selected from about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, and about 5.0%; and
wherein the sodium chloride is at a concentration selected from about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 146 mM, and about 150 mM. In some aspects, wherein the ASO comprises a nucleic acid sequence selected from SEQ ID NOs: 91-193

In certain aspects, the composition comprises:
(a) Extracellular vesicles comprising an ASO, wherein the ASO comprises a contiguous nucleotide sequence of 10 to 30 nucleotides in length that is complementary to a nucleic acid sequence within a STAT6 transcript;
(b) Sucrose at a concentration of about 73 mM to about 146 mM;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
(f) wherein the pH of the composition is about 7.2.

In some aspects, the composition comprises:
(a) Extracellular vesicles comprising an ASO, wherein the ASO comprises a contiguous nucleotide sequence of 10 to 30 nucleotides in length that is complementary to a nucleic acid sequence within a STAT6 transcript;
(b) Sucrose at a concentration of about 2.5% to about 5%;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
(f) wherein the pH of the composition is about 7.2.

In certain aspects, the composition comprises:
(a) Extracellular vesicles comprising an ASO, wherein the ASO comprises a nucleic acid sequence selected from SEQ ID NOs: 91-193;
(b) Sucrose at a concentration of about 73 mM to about 146 mM;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
(f) wherein the pH of the composition is about 7.2.

In some aspects, the composition comprises:
(a) Extracellular vesicles comprising an ASO, wherein the ASO comprises a nucleic acid sequence selected from SEQ ID NOs: 91-193;
(b) Sucrose at a concentration of about 2.5% to about 5%;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
(f) wherein the pH of the composition is about 7.2.

In certain aspects, the composition comprises:
(a) Extracellular vesicles comprising an ASO, wherein the ASO comprises a contiguous nucleotide sequence of 10 to 30 nucleotides in length that is complementary to a nucleic acid sequence within a STAT6 transcript;
(b) Sucrose at a concentration of about 146 mM;
(c) Sodium chloride at a concentration of about 50 mM;
(d) Potassium phosphate monobasic at a concentration of about 5 mM;
(e) Sodium phosphate dibasic at a concentration of about 15 mM,
(f) wherein the pH of the composition is about 7.2.

In some aspects, the composition comprises:
(a) Extracellular vesicles comprising an ASO, wherein the ASO comprises a contiguous nucleotide sequence of 10 to 30 nucleotides in length that is complementary to a nucleic acid sequence within a STAT6 transcript;
(b) Sucrose at a concentration of about 5%;
(c) Sodium chloride at a concentration of about 50 mM;
(d) Potassium phosphate monobasic at a concentration of about 5 mM;
(e) Sodium phosphate dibasic at a concentration of about 15 mM,
(f) wherein the pH of the composition is about 7.2.

In some aspects, the composition comprises:
(a) Extracellular vesicles comprising an ASO, wherein the ASO comprises a contiguous nucleotide sequence of 10 to 30 nucleotides in length that is complementary to a nucleic acid sequence within a STAT6 transcript;
(b) Sucrose at a concentration of about 5%;
(c) Sodium chloride at a concentration of about 100 mM;
(d) Potassium phosphate monobasic at a concentration of about 5 mM;
(e) Sodium phosphate dibasic at a concentration of about 15 mM,
(f) wherein the pH of the composition is about 7.2.

In some aspects, the composition comprises:
(a) Extracellular vesicles comprising an ASO, wherein the ASO comprises a contiguous nucleotide sequence of 10 to 30 nucleotides in length that is complementary to a nucleic acid sequence within a STAT6 transcript;
(b) Sucrose at a concentration of about 5%;
(c) Sodium chloride at a concentration of about 150 mM;
(d) Potassium phosphate monobasic at a concentration of about 5 mM;
(e) Sodium phosphate dibasic at a concentration of about 15 mM,
(f) wherein the pH of the composition is about 7.2.

In some aspects, the composition comprises:
(a) Extracellular vesicles comprising an ASO, wherein the ASO comprises a contiguous nucleotide sequence of 10 to 30 nucleotides in length that is complementary to a nucleic acid sequence within a STAT6 transcript;
(b) Sucrose at a concentration of about 2.5%;
(c) Sodium chloride at a concentration of about 50 mM;
(d) Potassium phosphate monobasic at a concentration of about 5 mM;
(e) Sodium phosphate dibasic at a concentration of about 15 mM,
(f) wherein the pH of the composition is about 7.2.

In some aspects, the ASO comprises a nucleic acid sequence selected from SEQ ID NOs: 91-93.

In certain aspects, the composition comprises:
(a) Extracellular vesicles comprising an ASO, wherein the ASO comprises a nucleic acid sequence selected from SEQ ID NOs: 91-193;
(b) Sucrose at a concentration of about 146 mM;
(c) Sodium chloride at a concentration of about 50 mM;
(d) Potassium phosphate monobasic at a concentration of about 5 mM;
(e) Sodium phosphate dibasic at a concentration of about 15 mM,
(f) wherein the pH of the composition is about 7.2.

In some aspects, the composition comprises:
(a) Extracellular vesicles comprising an ASO, wherein the ASO comprises a nucleic acid sequence selected from SEQ ID NOs: 91-193;
(b) Sucrose at a concentration of about 5%;
(c) Sodium chloride at a concentration of about 50 mM;
(d) Potassium phosphate monobasic at a concentration of about 5 mM;
(e) Sodium phosphate dibasic at a concentration of about 15 mM,
(f) wherein the pH of the composition is about 7.2.

In some aspects, the composition comprises:
(a) Extracellular vesicles comprising an ASO, wherein the ASO comprises a nucleic acid sequence selected from SEQ ID NOs: 91-193;
(b) Sucrose at a concentration of about 4.5%;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
(f) wherein the pH of the composition is about 7.2.

In some aspects, the composition comprises:
(a) Extracellular vesicles comprising an ASO, wherein the ASO comprises a nucleic acid sequence selected from SEQ ID NOs: 91-193;
(b) Sucrose at a concentration of about 4%;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
(f) wherein the pH of the composition is about 7.2.

In some aspects, the composition comprises:
(a) Extracellular vesicles comprising an ASO, wherein the ASO comprises a nucleic acid sequence selected from SEQ ID NOs: 91-193;
(b) Sucrose at a concentration of about 3.5%;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
(f) wherein the pH of the composition is about 7.2.

In some aspects, the composition comprises:
(a) Extracellular vesicles comprising an ASO, wherein the ASO comprises a nucleic acid sequence selected from SEQ ID NOs: 91-193;
(b) Sucrose at a concentration of about 3%;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
(f) wherein the pH of the composition is about 7.2.

In some aspects, the composition comprises:
(a) Extracellular vesicles comprising an ASO, wherein the ASO comprises a nucleic acid sequence selected from SEQ ID NOs: 91-193;
(b) Sucrose at a concentration of about 2.5%;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
(f) wherein the pH of the composition is about 7.2.

In certain aspects, the composition comprises:
(a) Extracellular vesicles comprising an ASO, wherein the ASO comprises the nucleic acid sequence GAAAGGTTCCGTCGGGC (SEQ ID NO: 144);
(b) Sucrose at a concentration of about 146 mM;
(c) Sodium chloride at a concentration of about 50 mM;
(d) Potassium phosphate monobasic at a concentration of about 5 mM;
(e) Sodium phosphate dibasic at a concentration of about 15 mM,
(f) wherein the pH of the composition is about 7.2.

In some aspects, the composition comprises:
(a) Extracellular vesicles comprising an ASO, wherein the ASO comprises the nucleic acid sequence GAAAGGTTCCGTCGGGC (SEQ ID NO: 144);
(b) Sucrose at a concentration of about 5%;
(c) Sodium chloride at a concentration of about 50 mM;
(d) Potassium phosphate monobasic at a concentration of about 5 mM;
(e) Sodium phosphate dibasic at a concentration of about 15 mM,
(f) wherein the pH of the composition is about 7.2.

In certain aspects, the composition comprises:
(a) Extracellular vesicles comprising an ASO, wherein the ASO comprises the nucleic acid sequence CTGAGTCGCTGAAGCGG (SEQ ID NO: 145);
(b) Sucrose at a concentration of about 146 mM;
(c) Sodium chloride at a concentration of about 50 mM;
(d) Potassium phosphate monobasic at a concentration of about 5 mM;
(e) Sodium phosphate dibasic at a concentration of about 15 mM,
(f) wherein the pH of the composition is about 7.2.

In some aspects, the composition comprises:
(a) Extracellular vesicles comprising an ASO, wherein the ASO comprises the nucleic acid sequence CTGAGTCGCTGAAGCGG (SEQ ID NO: 145);
(b) Sucrose at a concentration of about 5%;
(c) Sodium chloride at a concentration of about 50 mM;
(d) Potassium phosphate monobasic at a concentration of about 5 mM;
(e) Sodium phosphate dibasic at a concentration of about 15 mM,
(f) wherein the pH of the composition is about 7.2.

In certain aspects, the composition comprises:
(a) Extracellular vesicles comprising an ASO, wherein the ASO comprises the nucleic acid sequence GCCCTTGTACTTTTGCATAG (SEQ ID NO: 193);
(b) Sucrose at a concentration of about 146 mM;
(c) Sodium chloride at a concentration of about 50 mM;
(d) Potassium phosphate monobasic at a concentration of about 5 mM;
(e) Sodium phosphate dibasic at a concentration of about 15 mM,
(f) wherein the pH of the composition is about 7.2.

In some aspects, the composition comprises:
(a) Extracellular vesicles comprising an ASO, wherein the ASO comprises the nucleic acid sequence GCCCTTGTACTTTTGCATAG (SEQ ID NO: 193);
(b) Sucrose at a concentration of about 5%;
(c) Sodium chloride at a concentration of about 50 mM;
(d) Potassium phosphate monobasic at a concentration of about 5 mM;
(e) Sodium phosphate dibasic at a concentration of about 15 mM,
(f) wherein the pH of the composition is about 7.2.

In certain aspects, the composition comprises:
(a) Extracellular vesicles comprising an ASO, wherein the ASO comprises the nucleic acid sequence GCAAGATCCCGGATTCGGTC (SEQ ID NO: 185);
(b) Sucrose at a concentration of about 146 mM;
(c) Sodium chloride at a concentration of about 50 mM;
(d) Potassium phosphate monobasic at a concentration of about 5 mM;
(e) Sodium phosphate dibasic at a concentration of about 15 mM,
(f) wherein the pH of the composition is about 7.2.

In some aspects, the composition comprises:
(a) Extracellular vesicles comprising an ASO, wherein the ASO comprises the nucleic acid sequence GCAAGATCCCGGATTCGGTC (SEQ ID NO: 185);
(b) Sucrose at a concentration of about 5%;
(c) Sodium chloride at a concentration of about 50 mM;
(d) Potassium phosphate monobasic at a concentration of about 5 mM;
(e) Sodium phosphate dibasic at a concentration of about 15 mM,
(f) wherein the pH of the composition is about 7.2.

In some aspects, the composition is lyophilized.

### III. Extracellular Vesicles, e.g., Exosomes

Disclosed herein are modified EVs, e.g., exosomes, capable of regulating the immune system of a subject. The EVs, e.g., exosomes, useful in the present disclosure have been engineered to produce at least one exogenous biologically active moiety. In some aspects, an EV (e.g., exosome) comprises two exogenous biologically active moieties. In some aspects, an EV (e.g., exosome) comprises three exogenous biologically active moieties. In other aspects, an EV (e.g., exosome) comprises four exogenous biologically active moieties. In further aspects, an EV (e.g., exosome) comprises five or more exogenous biologically active moieties. In some aspects, an EV (e.g., exosome) comprises 6, 7, 8, 9, 10, 11, 12, 13, 14, 15, 16, 17, 18, 19, 20 or more exogenous biologically active moieties.

As described supra, EVs, e.g., exosomes, described herein are extracellular vesicles with a diameter between about 20-300 nm. In certain aspects, an EV, e.g., exosome, of the present disclosure has a diameter between about 20-290 nm, 20-280 nm, 20-270 nm, 20-260 nm, 20-250 nm, 20-240 nm, 20-230 nm, 20-220 nm, 20-210 nm, 20-200 nm, 20-190 nm, 20-180 nm, 20-170 nm, 20-160 nm, 20-150 nm, 20-140 nm, 20-130 nm, 20-120 nm, 20-110 nm, 20-100 nm, 20-90 nm, 20-80 nm, 20-70 nm, 20-60 nm, 20-50 nm, 20-40 nm, 20-30 nm, 30-300 nm, 30-290 nm, 30-280 nm, 30-270 nm, 30-260 nm, 30-250 nm, 30-240 nm, 30-230 nm, 30-220 nm, 30-210 nm, 30-200 nm, 30-190 nm, 30-180 nm, 30-170 nm, 30-160 nm, 30-150 nm, 30-140 nm, 30-130 nm, 30-120 nm, 30-110 nm, 30-100 nm, 30-90 nm, 30-80 nm, 30-70 nm, 30-60 nm, 30-50 nm, 30-40 nm, 40-300 nm, 40-290 nm, 40-280 nm, 40-270 nm, 40-260 nm, 40-250 nm, 40-240 nm, 40-230 nm, 40-220 nm, 40-210 nm, 40-200 nm, 40-190 nm, 40-180 nm, 40-170 nm, 40-160 nm, 40-150 nm, 40-140 nm, 40-130 nm, 40-120 nm, 40-110 nm, 40-100 nm, 40-90 nm, 40-80 nm, 40-70 nm, 40-60 nm, 40-50 nm, 50-300 nm, 50-290 nm, 50-280 nm, 50-270 nm, 50-260 nm, 50-250 nm, 50-240 nm, 50-230 nm, 50-220 nm, 50-210 nm, 50-200 nm, 50-190 nm, 50-180 nm, 50-170 nm, 50-160 nm, 50-150 nm, 50-140 nm, 50-130 nm, 50-120 nm, 50-110 nm, 50-100 nm, 50-90 nm, 50-80 nm, 50-70 nm, 50-60 nm, 60-300 nm, 60-290 nm, 60-280 nm, 60-270 nm, 60-260 nm, 60-250 nm, 60-240 nm, 60-230 nm, 60-220 nm, 60-210 nm, 60-200 nm, 60-190 nm, 60-180 nm, 60-170 nm, 60-160 nm, 60-150 nm, 60-140 nm, 60-130 nm, 60-120 nm, 60-110 nm, 60-100 nm, 60-90 nm, 60-80 nm, 60-70 nm, 70-300 nm, 70-290 nm, 70-280 nm, 70-270 nm, 70-260 nm, 70-250 nm, 70-240 nm, 70-230 nm, 70-220 nm, 70-210 nm, 70-200 nm, 70-190 nm, 70-180 nm, 70-170 nm, 70-160 nm, 70-150 nm, 70-140 nm, 70-130 nm, 70-120 nm, 70-110 nm, 70-100 nm, 70-90 nm, 70-80 nm, 80-300 nm, 80-290 nm, 80-280 nm, 80-270 nm, 80-260 nm, 80-250 nm, 80-240 nm, 80-230 nm, 80-220 nm, 80-210 nm, 80-200 nm, 80-190 nm, 80-180 nm, 80-170 nm, 80-160 nm, 80-150 nm, 80-140 nm, 80-130 nm, 80-120 nm, 80-110 nm, 80-100 nm, 80-90 nm, 90-300 nm, 90-290 nm, 90-280 nm, 90-270 nm, 90-260 nm, 90-250 nm, 90-240 nm, 90-230 nm, 90-220 nm, 90-210 nm, 90-200 nm, 90-190 nm, 90-180 nm, 90-170 nm, 90-160 nm, 90-150 nm, 90-140 nm, 90-130 nm, 90-120 nm, 90-110 nm, 90-100 nm, 100-300 nm, 110-290 nm, 120-280 nm, 130-270 nm, 140-260 nm, 150-250 nm, 160-240 nm, 170-230 nm, 180-220 nm, or 190-210 nm. The size of the EV, e.g., exosome, described herein can be measured according to methods described, infra.

In some aspects, an EV, e.g., exosome, of the present disclosure comprises a bi-lipid membrane (“EV, e.g., exosome, membrane”), comprising an interior surface and an exterior surface. In certain aspects, the interior surface faces the inner core (i.e., lumen) of the EV, e.g., exosome. In certain aspects, the exterior surface can be in contact with the endosome, the multivesicular bodies, or the membrane/cytoplasm of a producer cell or a target cell

In some aspects, the EV, e.g., exosome, membrane comprises lipids and fatty acids. In some aspects, the EV, e.g., exosome, membrane comprises phospholipids, glycolipids, fatty acids, sphingolipids, phosphoglycerides, sterols, cholesterols, and phosphatidylserines.

In some aspects, the EV, e.g., exosome, membrane comprises an inner leaflet and an outer leaflet. The composition of the inner and outer leaflet can be determined by transbilayer distribution assays known in the art, see, e.g., Kuypers et al., Biohim Biophys Acta 1985 819:170. In some aspects, the composition of the outer leaflet is between approximately 70-90% choline phospholipids, between approximately 0-15% acidic phospholipids, and between approximately 5-30% phosphatidylethanolamine. In some aspects, the composition of the inner leaflet is between approximately 15-40% choline phospholipids, between approximately 10-50% acidic phospholipids, and between approximately 30-60% phosphatidylethanolamine.

In some aspects, the EV, e.g., exosome, membrane comprises one or more polysaccharide, such as glycan.

In some aspects, the EV, e.g., exosome, membrane further comprises one or more scaffold moieties, which are capable of anchoring the multiple exogenous biologically active moieties to the EV, e.g., exosome, (e.g., either on the luminal surface or on the exterior surface). In some aspects, the scaffold moieties anchor or link at least one of the multiple exogenous biologically active moieties to the EV. In some aspects, the scaffold moieties anchor or link each of the multiple (e.g., at least two) exogenous biologically active moieties to the EV. In certain aspects, scaffold moieties are polypeptides (“exosome proteins”). In other aspects, scaffold moieties are non-polypeptide moieties. In some aspects, exosome proteins include various membrane proteins, such as transmembrane proteins, integral proteins and peripheral proteins, enriched on the exosome membranes. They can include various CD proteins, transporters, integrins, lectins, and cadherins. In certain aspects, a scaffold moiety (e.g., exosome protein) comprises Scaffold X. In further aspects, a scaffold moiety (e.g., exosome protein) comprises more than one Scaffold X moiety.

In some aspects, an EV, e.g., exosome, disclosed herein is capable of delivering one or more payload (e.g., a biologically active moiety) to a target. Accordingly, in certain aspects, an EV (e.g., exosome) comprises one, two, three, four, five or more different payloads. The payload is an agent that acts on a target (e.g., a target cell) that is contacted with the EV. Contacting can occur in vitro or in a subject. Non-limiting examples of payloads that can be introduced into an EV include agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, or siRNA), RNA binding proteins such as MS2, amino acids (e.g., amino acids comprising a detectable moiety or a toxin that disrupt translation), polypeptides (e.g., enzymes), lipids, carbohydrates, and small molecules (e.g., small molecule drugs and toxins). In some aspects, a payload comprises an exogenous biologically active moiety (e.g., those disclosed herein).

IIIA. Payload, e.g., Biologically Active Moiety

In some aspects, the payload comprises a biologically active moiety. In some aspects, the payload comprises a therapeutic molecule. In certain aspects a therapeutic molecule comprises an antigen, which is capable of inducing an immune response in a subject. In certain aspects, the antigen comprises a tumor antigen. Non-limiting examples of tumor antigens include: alpha-fetoprotein (AFP), carcinoembryonic antigen (CEA), epithelial tumor antigen (ETA), mucin 1 (MUC1), Tn-MUC1, mucin 16 (MUC16), tyrosinase, melanoma-associated antigen (MAGE), tumor protein p53 (p53), CD4, CD8, CD45, CD47, CD80, CD86, programmed death ligand 1 (PD-L1), programmed death ligand 2 (PD-L2), NY-ESO-1, PSMA, TAG-72, HER2, GD2, cMET, EGFR, Mesothelin, VEGFR, alpha-folate receptor, CE7R, IL-3, Cancer-testis antigen (CTA), MART-1 gp100, TNF-related apoptosis-inducing ligand, Brachyury (preferentially expressed antigen in melanoma (PRAME)), or combinations thereof. In further aspects, an antigen can comprise a neoantigen. As used herein, the term “neoantigen,” refers to antigens encoded by tumor-specific mutated genes. In some aspects, the antigen is derived from a bacterium, a virus, fungus, protozoa, or any combination thereof. In some aspects, the antigen is derived from an oncogenic virus. In further aspects, the antigen is derived from a group comprising: a Human Gamma herpes virus 4 (Epstein Barr virus), influenza A virus, influenza B virus, cytomegalovirus, Staphylococcus aureus, Mycobacterium tuberculosis, Chlamydia trachomatis, HIV-1, HIV-2, corona viruses (e.g., MERS-CoV and SARS CoV), filoviruses (e.g., Marburg and Ebola), Streptococcus pyogenes, Streptococcus pneumoniae, Plasmodia species (e.g., vivax and falciparum), Chikungunya virus, Human Papilloma virus (HPV), Hepatitis B, Hepatitis C, human herpes virus 8, herpes simplex virus 2 (HSV2), Klebsiella sp., Pseudomonas aeruginosa, Enterococcus sp., Proteus sp., Enterobacter sp., Actinobacter sp., coagulase-negative staphylococci (CoNS), Mycoplasma sp., or combinations thereof.

In some aspects, the antigen is derived from Mycobacterium tuberculosis to induce cellular and/or humoral immune response. In some aspects, the antigen comprises one or more epitopes of Mycobacterium tuberculosis (TB antigen). Various antigens are associated with Mycobacterium tuberculosis infection, including ESAT-6, TB10.4, CFP10, Rv2031 (hspX), Rv2654c (TB7.7), and Rv1038c (EsxJ). See, e.g., Lindestam et al., J. Immunol. 188(10):5020-31 (2012), which is incorporated herein in its entirety.

In some aspects, an antigen comprises a self-antigen. As used herein, the term “self-antigen” refers to an antigen that is expressed by a host cell or tissue.

In some aspects, the therapeutic molecule comprises an antibody or antigen-binding fragment thereof. In some aspects, the therapeutic molecule comprises at least 2, at least 3, at least 4, or at least 5 antibodies or antigen-binding fragments thereof. In some aspects, the biologically active moiety is an antibody that targets cell surface proteins. In some aspects, the antibody or antigen-binding fragment thereof comprises a scFv, scFab, scFab-Fc, nanobody, or any combination thereof. In some aspects, the antibody or antigen-binding fragment thereof comprises an agonist antibody, blocking antibody, a targeting antibody, a fragment thereof, or a combination thereof. In some aspects, the agonist antibody is a CD40L agonist. In some aspects, the blocking antibody binds a target protein selected from programmed death 1 (PD-1), programmed death ligand 1 (PD-L1), cytotoxic T-lymphocyte-associated protein 4, and any combination thereof. In some aspects, the targeting antibody binds a CD3 and/or CD19. In some aspects, the EV, e.g., exosome, comprises an anti-IL12 antibody or an antigen binding fragment thereof and an anti-CD40L antibody or antigen binding fragment thereof. In some aspects, the EV, e.g, exosome comprises an BITES, e.g., an anti-CD3 antibody and an anti-cancer agent, e.g., an anti-CD19 antibody.

In some aspects, the therapeutic molecule comprises a fragment of an antibody e.g., scFv, (scFv)₂, Fab, Fab′, and F(ab′)₂, F(ab1)₂, Fv, dAb, or Fd targeting antigens including CD33, ICAM4, CD40, CDLEC9A, DEC205, and TfR, and any combination thereof.

In some aspects, therapeutic molecule is a blood clotting factor, including FVIII.

In some aspects, the therapeutic molecule is a targeting peptide, including cysteine knot peptides.

In some aspects, the therapeutic molecule is an enzyme including Cas9 and zinc finger nucleases, CD39, CD73, and lysosomal acid glucosylceramidase.

In some aspects, the therapeutic molecule is a protein dimerization system, including FRB-FKBP.

As described supra, EVs, e.g., exosomes, of the present disclosure can comprise an adjuvant. In some aspects, EVs (e.g., exosome) disclosed herein comprises one, two, three, four, five or more different adjuvants. As used herein, the term “adjuvant” refers to any substance that enhances the therapeutic effect of the payload (e.g., increasing an immune response to the antigen). Accordingly, EVs, e.g., exosomes, described herein are capable of increasing an immune response to an antigen by at least about 5%, at least about 10%, at least about 20%, at least about 30%, at least about 40%, at least about 50%, at least about 60%, at least about 70%, at least about 80%, at least about 90%, at least about 100% or more, compared to a reference (e.g., corresponding EV without the adjuvant or a non-EV delivery vehicle comprising an antigen and adjuvant). Non-limiting examples of adjuvants include: Stimulator of Interferon Genes (STING) agonist, a toll-like receptor (TLR) agonist, an inflammatory mediator, and combinations thereof.

In some aspects, an adjuvant induces the activation of a cytosolic pattern recognition receptor. Non-limiting examples of cytosolic pattern recognition receptor includes: stimulator of interferon genes (STING), retinoic acid-inducible gene I (RIG-1), Melanoma Differentiation-Associated protein 5 (MDAS), Nucleotide-binding oligomerization domain, Leucine rich Repeat and Pyrin domain containing (NLRP), inflammsomes, or combinations thereof. In certain aspects, an adjuvant is a STING agonist. Stimulator of Interferon Genes (STING) is a cytosolic sensor of cyclic dinucleotides that is typically produced by bacteria. Upon activation, it leads to the production of type I interferons and initiates an immune response. In certain aspects, the STING agonist comprises a cyclic dinucleotide STING agonist or a non-cyclic dinucleotide STING agonist.

Cyclic purine dinucleotides such as, but not limited to, cGMP, cyclic di-GMP (c-di-GMP), cAMP, cyclic di-AMP (c-di-AMP), cyclic-GMP-AMP (cGAMP), cyclic di-IMP (c-di-IMP), cyclic AMP-IMP (cAIMP), and any analogue thereof, are known to stimulate or enhance an immune or inflammation response in a patient. The CDNs can have 2′2′, 2′3′, 2′5′, 3′3′, or 3′5′ bonds linking the cyclic dinucleotides, or any combination thereof.

Cyclic purine dinucleotides can be modified via standard organic chemistry techniques to produce analogues of purine dinucleotides. Suitable purine dinucleotides include, but are not limited to, adenine, guanine, inosine, hypoxanthine, xanthine, isoguanine, or any other appropriate purine dinucleotide known in the art. The cyclic dinucleotides can be modified analogues. Any suitable modification known in the art can be used, including, but not limited to, phosphorothioate, biphosphorothioate, fluorinate, and difluorinate modifications.

Non cyclic dinucleotide agonists can also be used, such as 5,6-Dimethylxanthenone-4-acetic acid (DMXAA), or any other non-cyclic dinucleotide agonist known in the art.

Non-limiting examples of STING agonists that can be used with the present disclosure include: DMXAA, STING agonist-1, ML RR-S2 CDA, ML RR-S2c-di-GMP, ML-RR-S2 cGAMP, 2′3′-c-di-AM(PS)2, 2′3′-cGAMP, 2′3′-cGAMPdFHS, 3′3′-cGAMP, 3′3′-cGAMPdFSH, cAIMP, cAIM(PS)2, 3′3′-cAIMP, 3′3′-cAIMPdFSH, 2′2′-cGAMP, 2′3′-cGAM(PS)2, 3′3′-cGAMP, and combinations thereof. Non-limiting examples of the STING agonists can be found at U.S. Pat. No. 9,695,212, WO 2014/189805 A1, WO 2014/179335 A1, WO 2018/100558 A1, U.S. Pat. No. 10,011,630 B2, WO 2017/027646 A1, WO 2017/161349 A1, and WO 2016/096174 A1, each of which is incorporated by reference in its entirety.

In some aspects, the STING agonist useful for the present disclosure comprises a compound or a pharmaceutically acceptable salt thereof disclosed in WO 2016/096174, WO 2016/096174A1, WO 2014/093936, WO 2014/189805, WO 2015/077354, the content of which is incorporated herein by reference in its entirety. See also Cell reports 11, 1018-1030 (2015).

In some aspects, the STING agonist useful for the present disclosure comprises c-di-AMP, c-di-GMP, c-di-IMP, c-AMP-GMP, c-AMP-IMP, and c-GMP-IMP, described in WO 2013/185052 and Sci. Transl. Med. 283,283ra52 (2015), which are incorporated herein by reference in their entireties.

In some aspects, the STING agonist useful for the present disclosure comprises a compound or a pharmaceutically acceptable salt thereof disclosed in WO 2014/189806, WO 2015/185565, WO 2014/179760, WO 2014/179335, WO 2015/017652, WO 2016/096577, WO 2016/120305, WO 2016/145102, WO 2017/027646, WO 2017/075477, WO 2017/027645, WO 2018/100558, WO 2017/175147, or WO 2017/175156, each content of which is incorporated herein by reference in its entirety.

In some aspects, the STING agonist useful for the present disclosure is CL606, CL611, CL602, CL655, CL604, CL609, CL614, CL656, CL647, CL626, CL629, CL603, CL632, CL633, CL659, or a pharmaceutically acceptable salt thereof. In some aspects, the STING agonist useful for the present disclosure is CL606 or a pharmaceutically acceptable salt thereof. In some aspects, the STING agonist useful for the present disclosure is CL611 or a pharmaceutically acceptable salt thereof. In some aspects, the STING agonist useful for the present disclosure is CL602 or a pharmaceutically acceptable salt thereof. In some aspects, the STING agonist useful for the present disclosure is CL655 or a pharmaceutically acceptable salt thereof. In some aspects, the STING agonist useful for the present disclosure is CL604 or a pharmaceutically acceptable salt thereof. In some aspects, the STING agonist useful for the present disclosure is CL609 or a pharmaceutically acceptable salt thereof. In some aspects, the STING agonist useful for the present disclosure is CL614 or a pharmaceutically acceptable salt thereof. In some aspects, the STING agonist useful for the present disclosure is CL647 or a pharmaceutically acceptable salt thereof. In some aspects, the STING agonist useful for the present disclosure is CL626 or a pharmaceutically acceptable salt thereof. In some aspects, the STING agonist useful for the present disclosure is CL629 or a pharmaceutically acceptable salt thereof. In some aspects, the STING agonist useful for the present disclosure is CL603 or a pharmaceutically acceptable salt thereof. In some aspects, the STING agonist useful for the present disclosure is CL632 or a pharmaceutically acceptable salt thereof. In some aspects, the STING agonist useful for the present disclosure is CL633 or a pharmaceutically acceptable salt thereof. In some aspects, the STING agonist useful for the present disclosure is CL659 or a pharmaceutically acceptable salt thereof.

In some aspects, the STING agonist useful for the present disclosure is CL656 or a pharmaceutically acceptable salt thereof. In some aspects, the STING agonist useful for the present disclosure is isomer A of CL656 or a pharmaceutically acceptable salt thereof. In some aspects, the STING agonist useful for the present disclosure is isomer B of CL656 or a pharmaceutically acceptable salt thereof. In some aspects, the STING agonist useful for the present disclosure is isomer C of CL656 or a pharmaceutically acceptable salt thereof. In some aspects, the STING agonist useful for the present disclosure is isomer D of CL656 or a pharmaceutically acceptable salt thereof.

The STING agonists of the present disclosure can also be modified to increase or control encapsulation of the agonist in an extracellular vesicle or exosome. This modification can include the addition of a lipid binding tag by treating the agonist with a chemical or enzyme, or by physically or chemically altering the polarity or charge of the STING agonist. The STING agonist can be modified by a single treatment, or by a combination of treatments, e.g., adding a lipid binding tag only, or adding a lipid binding tag and altering the polarity. The previous example is meant to be a non-limiting illustrative instance. It is contemplated that any combination of modifications can be practiced in the present disclosure. The modification can increase or control encapsulation of the agonist in the exosome by between 2-fold and 10,000 fold, between 10-fold and 1,000 fold, or between 100-fold and 500-fold compared to encapsulation of an unmodified agonist. The modification can increase encapsulation of the agonist in the exosome by at least 2-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, 6000-fold, 7000-fold, 8000-fold, 9000-fold, or 10,000-fold compared to encapsulation of an unmodified agonist.

In some aspects, one or more exogenous biologically active moieties, e.g., an adjuvant, is a TLR agonist. Non-limiting examples of TLR agonists include: TLR2 agonist (e.g., lipoteichoic acid, atypical LPS, MALP-2 and MALP-404, OspA, porin, LcrV, lipomannan, GPI anchor, lysophosphatidylserine, lipophosphoglycan (LPG), glycophosphatidylinositol (GPI), zymosan, hsp60, gH/gL glycoprotein, hemagglutinin), a TLR3 agonist (e.g., double-stranded RNA, e.g., poly(I.C)), a TLR4 agonist (e.g., lipopolysaccharides (LPS), lipoteichoic acid, β-defensin 2, fibronectin EDA, HMGB1, snapin, tenascin C), a TLR5 agonist (e.g., flagellin), a TLR6 agonist, a TLR7/8 agonist (e.g., single-stranded RNA, CpG-A, Poly G10, Poly G3, Resiquimod), a TLR9 agonist (e.g., unmethylated CpG DNA), and combinations thereof. Non-limiting examples of TLR agonists can be found at WO2008115319A2, US20130202707A1, US20120219615A1, US20100029585A1, WO2009030996A1, WO2009088401A2, and WO2011044246A1, each of which are incorporated by reference in its entirety.

In some aspects, one or more exogenous biologically active moieties, e.g., an adjuvant, is an inflammatory mediator.

In some aspects, an EV, e.g., exosome, of the present disclosure can comprise one or more exogenous biologically active moieties, e.g., immune modulators. In certain aspects, the one or more immune modulators are expressed in combination with other active biological molecules, e.g., those disclosed herein. In some aspects, the one or more immune modulators can be expressed on the surface (e.g., exterior surface or luminal surface) or in the lumen of the EV, e.g., exosome. Accordingly, in certain aspects, the one or more immune modulators are linked to a scaffold moiety (e.g., Scaffold X) on the exterior surface of the EV, e.g., exosome or on the luminal surface of the EV, e.g., exosome. In further aspects, the one or more immune modulators are in the lumen of the exosome (i.e., not linked to Scaffold X). In certain aspects, the immune modulator is a polynucleotide. In some of these aspects, the polynucleotide includes, but is not limited to, an mRNA, a miRNA, an siRNA, an antisense RNA, an shRNA, a lncRNA, and a dsDNA. In some aspects, the immune modulator is a protein, a peptide, a glycolipid, or a glycoprotein.

In some aspects, an EV (e.g., exosome) formulated for the present composition comprises IL-12. In some aspects, an EV (e.g., exosome) formulated for the present composition comprises CD40L. In some aspects, an EV (e.g., exosome) formulated for the present composition comprises FLT3L. In some aspects, an EV (e.g., exosome) formulated for the present composition comprises IL-12 and CD40L. In other aspects, an EV (e.g., exosome) comprises IL-12, CD40L, and FLT3L. In some aspects, the IL-12 comprises an amino acid sequence at least about 80%, at least about 85%, at least about 90%, at least about 95%, at least about 96%, at least about 97%, at least about 98%, or at least about 99% identical to the amino acid sequence set forth in SEQ ID NO: 11, 12, or 13 (Table 1A). In certain aspects, the IL-12 comprises the amino acid sequence set forth in SEQ ID NO: 11. In certain aspects, the IL-12 comprises the amino acid sequence set forth in SEQ ID NO: 12. In some aspects, the IL-12 comprises an amino acid sequence at least about 80%, at least about 85%, at least about 90%, at least about 95%, at least about 96%, at least about 97%, at least about 98%, or at least about 99% identical to the amino acid sequence set forth in SEQ ID NO: 13. In certain aspects, the IL-12 comprises the amino acid sequence set forth in SEQ ID NO: 13.

TABLE 1A
IL-12 Amino Acid Sequences.

| | |
|---|---|
| Human IL- | MCPARSLLLVATLVLLDHLSLARNLPVATPDPGMFPCLHHSQNLLRAVSNMLQKAR |
| 12A | QTLEFYPCTSEEIDHEDITKDKTSTVEACLPLELTKNESCLNSRETSFITNGSCLA |
| (p35)(signal | SRKTSFMMALCLSSIYEDLKMYQVEFKTMNAKLLMDPKRQIFLDQNMLAVIDELMQ |
| peptide) | ALNFNSETVPQKSSLEEPDFYKTKIKLCILLHAFRIRAVTIDRVMSYLNAS |
| SEQ ID NO: | |
| 11 | |
| Human IL- | MCHQQLVISWFSLVFLASPLVAIWELKKDVYVVELDWYPDAPGEMVVLTCDTPEED |
| 12B (p40) | GITWTLDQSSEVLGSGKTLTIQVKEFGDAGQYTCHKGGEVLSHSLLLLHKKEDGIW |
| (signal | STDILKDQKEPKNKTFLRCEAKNYSGRFTCWWLTTISTDLTFSVKSSRGSSDPQGV |
| peptide) | TCGAATLSAERVRGDNKEYEYSVECQEDSACPAAEESLPIEVMVDAVHKLKYENYT |
| SEQ ID NO: | SSFFIRDIIKPDPPKNLQLKPLKNSRQVEVSWEYPDTWSTPHSYFSLTFCVQVQGK |
| 12 | SKREKKDRVFTDKTSATVICRKNASISVRAQDRYYSSSWSEWASVPCS |
| IL-12 Fusion | MCHQQLVISWFSLVFLASPLVAIWELKKDVYVVELDWYPDAPGEMVVLTCDTPEED |
| (signal | GITWTLDQSSEVLGSGKTLTIQVKEFGDAGQYTCHKGGEVLSHSLLLLHKKEDGIW |
| peptide-p40- | STDILKDQKEPKNKTFLRCEAKNYSGRFTCWWLTTISTDLTFSVKSSRGSSDPQGV |
| linker-p35) | TCGAATLSAERVRGDNKEYEYSVECQEDSACPAAEESLPIEVMVDAVHKLKYENYT |
| SEQ ID NO: | SSFFIRDIIKPDPPKNLQLKPLKNSRQVEVSWEYPDTWSTPHSYFSLTFCVQVQGK |
| 13 | SKREKKDRVFTDKTSATVICRKNASISVRAQDRYYSSSWSEWASVPCSGGSGGGSG |
| | GGGSGGGGSGGGSGGRNLPVATPDPGMFPCLHHSQNLLRAVSNMLQKARQTLEFYP |
| | CTSEEIDHEDITKDKTSTVEACLPLELTKNESCLNSRETSFITNGSCLASRKTSFM |
| | MALCLSSIYEDLKMYQVEFKTMNAKLLMDPKRQIFLDQNMLAVIDELMQALNENSE |
| | TVPQKSSLEEPDFYKTKIKLCILLHAFRIRAVTIDRVMSYLNAS |

In some aspects, the IL-12 moiety comprises a p35 polypeptide or a fragment thereof. In some aspects, the IL-12 comprises an amino acid sequence having at least about 50%, at least about 60%, at least about 70%, at least about 80%, at least about 85%, at least about 90%, at least about 95%, at least about 96%, at least about 97%, at least about 98%, at least about 99%, or about 100% sequence identity to SEQ ID NO: 11. In some aspects, the IL-12 comprises an amino acid sequence having at least about 90% sequence identity to SEQ ID NO: 11. In some aspects, the IL-12 comprises an amino acid sequence having at least about 95% sequence identity to SEQ ID NO: 11. In some aspects, the IL-12 comprises an amino acid sequence having at least about 96% sequence identity to SEQ ID NO: 11. In some aspects, the IL-12 comprises an amino acid sequence having at least about 97% sequence identity to SEQ ID NO: 11. In some aspects, the IL-12 comprises an amino acid sequence having at least about 98% sequence identity to SEQ ID NO: 11. In some aspects, the IL-12 comprises an amino acid sequence having at least about 99% sequence identity to SEQ ID NO: 11. In some aspects, the IL-12 comprises the amino acid sequence set forth in SEQ ID NO: 11. In some aspects, the IL-12 moiety lacks a signal peptide (see Table 1A).

In some aspects, the IL-12 moiety comprises a p40 polypeptide or a fragment thereof. In some aspects, the IL-12 comprises an amino acid sequence having at least about 50%, at least about 60%, at least about 70%, at least about 80%, at least about 85%, at least about 90%, at least about 95%, at least about 96%, at least about 97%, at least about 98%, at least about 99%, or about 100% sequence identity to SEQ ID NO: 12. In some aspects, the IL-12 comprises an amino acid sequence having at least about 90% sequence identity to SEQ ID NO: 12. In some aspects, the IL-12 comprises an amino acid sequence having at least about 95% sequence identity to SEQ ID NO: 12. In some aspects, the IL-12 comprises an amino acid sequence having at least about 96% sequence identity to SEQ ID NO: 12. In some aspects, the IL-12 comprises an amino acid sequence having at least about 97% sequence identity to SEQ ID NO: 12. In some aspects, the IL-12 comprises an amino acid sequence having at least about 98% sequence identity to SEQ ID NO: 12. In some aspects, the IL-12 comprises an amino acid sequence having at least about 99% sequence identity to SEQ ID NO: 12. In some aspects, the IL-12 comprises the amino acid sequence set forth in SEQ ID NO: 3. In some aspects, the IL-12 moiety lacks a signal peptide (see Table 1A).

In some aspects, the IL-12 moiety comprises a p35 polypeptide or a fragment thereof and a p40 polypeptide or a fragment thereof. In some aspects, IL-12 moiety comprises a single polypeptide, wherein the p35 polypeptide or a fragment thereof is linked to the p40 polypeptide or a fragment thereof. In some aspects, the p35 polypeptide or a fragment thereof is linked to the p40 polypeptide or a fragment thereof by a linker. In some aspects, the linker is a peptide linker. In some aspects, the linker comprises one or more amino acids. In some aspects, the linker comprises a Gly-Ser (GS) linker. In some aspects, the GS linker comprises (G4S)ₙ, wherein n is an integer between 1 and 10. In some aspects, the GS linker comprises (G3S)ₙ, wherein n is an integer between 1 and 10.

In certain aspects, the IL-12 moiety comprises an amino acid sequence having at least about 50%, at least about 60%, at least about 70%, at least about 80%, at least about 85%, at least about 90%, at least about 95%, at least about 96%, at least about 97%, at least about 98%, at least about 99%, or about 100% sequence identity to SEQ ID NO: 13. In some aspects, the IL-12 comprises an amino acid sequence having at least about 90% sequence identity to SEQ ID NO: 13. In some aspects, the IL-12 comprises an amino acid sequence having at least about 95% sequence identity to SEQ ID NO: 13. In some aspects, the IL-12 comprises an amino acid sequence having at least about 96% sequence identity to SEQ ID NO: 13. In some aspects, the IL-12 comprises an amino acid sequence having at least about 97% sequence identity to SEQ ID NO: 13. In some aspects, the IL-12 comprises an amino acid sequence having at least about 98% sequence identity to SEQ ID NO: 13. In some aspects, the IL-12 comprises an amino acid sequence having at least about 99% sequence identity to SEQ ID NO: 13. In some aspects, the IL-12 comprises the amino acid sequence set forth in SEQ ID NO: 13. In some aspects, the IL-12 consists of the amino acid sequence set forth in SEQ ID NO: 13. In some aspects, the IL-12 consists essentially of the amino acid sequence set forth in SEQ ID NO: 13. In some aspects, the IL-12 moiety lacks a signal peptide (see Table RA).

In some aspects, an EV (e.g., exosome) formulated for the present composition comprises an antisense oligonucleotide (ASO) which comprises a contiguous nucleotide sequence of 10 to 30 nucleotides in length that is complementary to a nucleic acid sequence within a STAT6 transcript (SEQ ID NO: 23; Table 1B). In some aspects, the ASO comprises a nucleic acid sequence selected from SEQ ID NOs: 91-193. In some aspects, the ASO comprises the nucleic acid sequence GAAAGGTTCCGTCGGGC (SEQ ID NO: 144). In some aspects, the ASO comprises the nucleic acid sequence CTGAGTCGCTGAAGCGG (SEQ ID NO: 145). In some aspects, the ASO comprises the nucleic acid sequence GCCCTTGTACTTTTGCATAG (SEQ ID NO: 193). In some aspects, the ASO comprises the nucleic acid sequence GCAAGATCCCGGATTCGGTC (SEQ ID NO: 185).

TABLE 1B
STAT6 mRNA and Protein Sequences
STAT6 mRNA Sequence

GGGGCAGCCACTGCTTACACTGAAGAGGGAGGACGGGAGAGGAGTGTGTGTGTGTGTGTGTGTGTGTGTGTGTATG
TATGTGTGTGCTTTATCTTATTTTTCTTTTTGGTGGTGGTGGTGGAAGGGGGGAGGTGCTAGCAGGGCCAGCCTTG
AACTCGCTGGACAGAGCTACAGACCTATGGGGCCTGGAAGTGCCCGCTGAGAAAGGGAGAAGACAGCAGAGGGGTT
GCCGAGGCAACCTCCAAGTCCCAGATCATGTCTCTGTGGGGTCTGGTCTCCAAGATGCCCCCAGAAAAAGTGCAGC
GGCTCTATGTCGACTTTCCCCAACACCTGCGGCATCTTCTGGGTGACTGGCTGGAGAGCCAGCCCTGGGAGTTCCT
GGTCGGCTCCGACGCCTTCTGCTGCAACTTGGCTAGTGCCCTACTTTCAGACACTGTCCAGCACCTTCAGGCCTCG
GTGGGAGAGCAGGGGGAGGGGAGCACCATCTTGCAACACATCAGCACCCTTGAGAGCATATATCAGAGGGACCCCC
TGAAGCTGGTGGCCACTTTCAGACAAATACTTCAAGGAGAGAAAAAAGCTGTTATGGAACAGTTCCGCCACTTGCC
AATGCCTTTCCACTGGAAGCAGGAAGAACTCAAGTTTAAGACAGGCTTGCGGAGGCTGCAGCACCGAGTAGGGGAG
ATCCACCTTCTCCGAGAAGCCCTGCAGAAGGGGGCTGAGGCTGGCCAAGTGTCTCTGCACAGCTTGATAGAAACTC
CTGCTAATGGGACTGGGCCAAGTGAGGCCCTGGCCATGCTACTGCAGGAGACCACTGGAGAGCTAGAGGCAGCCAA
AGCCCTAGTGCTGAAGAGGATCCAGATTTGGAAACGGCAGCAGCAGCTGGCAGGGAATGGCGCACCGTTTGAGGAG
AGCCTGGCCCCACTCCAGGAGAGGTGTGAAAGCCTGGTGGACATTTATTCCCAGCTACAGCAGGAGGTAGGGGCGG
CTGGTGGGGAGCTTGAGCCCAAGACCCGGGCATCGCTGACTGGCCGGCTGGATGAAGTCCTGAGAACCCTCGTCAC
CAGTTGCTTCCTGGTGGAGAAGCAGCCCCCCCAGGTACTGAAGACTCAGACCAAGTTCCAGGCTGGAGTTCGATTC
CTGTTGGGCTTGAGGTTCCTGGGGGCCCCAGCCAAGCCTCCGCTGGTCAGGGCCGACATGGTGACAGAGAAGCAGG
CGCGGGAGCTGAGTGTGCCTCAGGGTCCTGGGGCTGGAGCAGAAAGCACTGGAGAAATCATCAACAACACTGTGCC
CTTGGAGAACAGCATTCCTGGGAACTGCTGCTCTGCCCTGTTCAAGAACCTGCTTCTCAAGAAGATCAAGCGGTGT
GAGCGGAAGGGCACTGAGTCTGTCACAGAGGAGAAGTGCGCTGTGCTCTTCTCTGCCAGCTTCACACTTGGCCCCG
GCAAACTCCCCATCCAGCTCCAGGCCCTGTCTCTGCCCCTGGTGGTCATCGTCCATGGCAACCAAGACAACAATGC
CAAAGCCACTATCCTGTGGGACAATGCCTTCTCTGAGATGGACCGCGTGCCCTTTGTGGTGGCTGAGCGGGTGCCC
TGGGAGAAGATGTGTGAAACTCTGAACCTGAAGTTCATGGCTGAGGTGGGGACCAACCGGGGGCTGCTCCCAGAGC
ACTTCCTCTTCCTGGCCCAGAAGATCTTCAATGACAACAGCCTCAGTATGGAGGCCTTCCAGCACCGTTCTGTGTC
CTGGTCGCAGTTCAACAAGGAGATCCTGCTGGGCCGTGGCTTCACCTTTTGGCAGTGGTTTGATGGTGTCCTGGAC
CTCACCAAACGCTGTCTCCGGAGCTACTGGTCTGACCGGCTGATCATTGGCTTCATCAGCAAACAGTACGTTACTA
GCCTTCTTCTCAATGAGCCCGACGGAACCTTTCTCCTCCGCTTCAGCGACTCAGAGATTGGGGGCATCACCATTGC
CCATGTCATCCGGGGCCAGGATGGCTCTCCACAGATAGAGAACATCCAGCCATTCTCTGCCAAAGACCTGTCCATT
CGCTCACTGGGGGACCGAATCCGGGATCTTGCTCAGCTCAAAAATCTCTATCCCAAGAAGCCCAAGGATGAGGCTT
TCCGGAGCCACTACAAGCCTGAACAGATGGGTAAGGATGGCAGGGGTTATGTCCCAGCTACCATCAAGATGACCGT
GGAAAGGGACCAACCACTTCCTACCCCAGAGCTCCAGATGCCTACCATGGTGCCTTCTTATGACCTTGGAATGGCC
CCTGATTCCTCCATGAGCATGCAGCTTGGCCCAGATATGGTGCCCCAGGTGTACCCACCACACTCTCACTCCATCC
CCCCGTATCAAGGCCTCTCCCCAGAAGAATCAGTCAACGTGTTGTCAGCCTTCCAGGAGCCTCACCTGCAGATGCC
CCCCAGCCTGGGCCAGATGAGCCTGCCCTTTGACCAGCCTCACCCCCAGGGCCTGCTGCCGTGCCAGCCTCAGGAG
CATGCTGTGTCCAGCCCTGACCCCCTGCTCTGCTCAGATGTGACCATGGTGGAAGACAGCTGCCTGAGCCAGCCAG
TGACAGCGTTTCCTCAGGGCACTTGGATTGGTGAAGACATATTCCCTCCTCTGCTGCCTCCCACTGAACAGGACCT
CACTAAGCTTCTCCTGGAGGGGCAAGGGGAGTCGGGGGGAGGGTCCTTGGGGGCACAGCCCCTCCTGCAGCCCTCC
CACTATGGGCAATCTGGGATCTCAATGTCCCACATGGACCTAAGGGCCAACCCCAGTTGGTGATCCCAGCTGGAGG
GAGAACCCAAAGAGACAGCTCTTCTACTACCCCCACAGACCTGCTCTGGACACTTGCTCATGCCCTGCCAAGCAGC
AGATGGGGAGGGTGCCCTCCTATCCCCACCTACTCCTGGGTCAGGAGGAAAAGACTAACAGGAGAATGCACAGTGG
GTGGAGCCAATCCACTCCTTCCTTTCTATCATTCCCCTGCCCACCTCCTTCCAGCACTGACTGGAAGGGAAGTTCA
GGCTCTGAGACACACCCCAACATGCCTGCACCTGCAGCGCGCACACGCACGCACACACACATACAGAGCTCTCTGA
GGGTGATGGGGCTGAGCAGGAGGGGGGCTGGGTAAGAGCACAGGTTAGGGCATGGAAGGCTTCTCCGCCCATTCTG
ACCCAGGGCCTAGGACGGATAGGCAGGAACATACAGACACATTTACACTAGAGGCCAGGGATAGAGGATATTGGGT
CTCAGCCCTAGGGGAATGGGAAGCAGCTCAAGGGACCCTGGGTGGGAGCATAGGAGGGGTCTGGACATGTGGTTAC
TAGTACAGGTTTTGCCCTGATTAAAAAATCTCCCAAAGCCCCAAATTCCTGTTAGCCAGGTGGAGGCTTCTGATAC
GTGTATGAGACTATGCAAAAGTACAAGGGCTGAGATTCTTCGTGTATAGCTGTGTGAACGTGTATGTACCTAGGAT
ATGTTAAATGTATAGCTGGCACCTTAGTTGCATGACCACATAGAACATGTGTCTATCTGCTTTTGCCTACGTGACA
ACACAAATTTGGGAGGGTGAGACACTGCACAGAAGACAGCAGCAAGTGTGCTGGCCTCTCTGACATATGCTAACCC
CCAAATACTCTGAATTTGGAGTCTGACTGTGCCCAAGTGGGTCCAAGTGGCTGTGACATCTACGTATGGCTCCACA
CCTCCAATGCTGCCTGGGAGCCAGGGTGAGAGTCTGGGTCCAGGCCTGGCCATGTGGCCCTCCAGTGTATGAGAGG
GCCCTGCCTGCTGCATCTTTTCTGTTGCCCCATCCACCGCCAGCTTCCCTTCACTCCCCTATCCCATTCTCCCTCT
CAAGGCAGGGGTCATAGATCCTAAGCCATAAAATAAATTTTATTCCAAAATAACAAAATAAATAATCTACTGTACA
CAATCTGAAAA (SEQ ID NO: 23)

IIIB. Scaffold Moieties, e.g., Scaffold X or Scaffold Y

In some aspects, EVs, e.g., exosomes, of the present disclosure comprise a membrane modified in its composition. For example, their membrane compositions can be modified by changing the protein, lipid, or glycan content of the membrane.

In some aspects, the surface-engineered EVs, e.g., exosomes, are generated by chemical and/or physical methods, such as PEG-induced fusion and/or ultrasonic fusion. In other aspects, the surface-engineered EVs, e.g., exosomes, are generated by genetic engineering. EVs, e.g., exosomes, produced from a genetically-modified producer cell or a progeny of the genetically-modified cell can contain modified membrane compositions. In some aspects, surface-engineered EVs, e.g., exosomes, have scaffold moiety (e.g., exosome protein, e.g., Scaffold X) at a higher or lower density (e.g., higher number) or include a variant or a fragment of the scaffold moiety.

For example, surface (e.g., Scaffold X)-engineered EVs, can be produced from a cell (e.g., HEK293 cells) transformed with an exogenous sequence encoding a scaffold moiety (e.g., exosome proteins, e.g., Scaffold X) or a variant or a fragment thereof. EVs including scaffold moiety expressed from the exogenous sequence can include modified membrane compositions.

Various modifications or fragments of the scaffold moiety can be used for the aspects of the present disclosure. For example, scaffold moiety modified to have enhanced affinity to a binding agent can be used for generating surface-engineered EV that can be purified using the binding agent. Scaffold moieties modified to be more effectively targeted to EVs and/or membranes can be used. Scaffold moieties modified to comprise a minimal fragment required for specific and effective targeting to exosome membranes can be also used.

Non-limiting examples of Scaffold moieties include: prostaglandin F2 receptor negative regulator (PTGFRN); basigin (BSG); immunoglobulin superfamily member 2 (IGSF2); immunoglobulin superfamily member 3 (IGSF3); immunoglobulin superfamily member 8 (IGSF8); integrin beta-1 (ITGB1); integrin alpha-4 (ITGA4); 4F2 cell-surface antigen heavy chain (SLC3A2); and a class of ATP transporter proteins (ATP1A1, ATP1A2, ATP1A3, ATP1A4, ATP1B3, ATP2B1, ATP2B2, ATP2B3, ATP2B). In certain aspects, Scaffold moieties is a whole protein. In other aspects, Scaffold moieties is a protein fragment (e.g., functional fragment).

In other aspects, the scaffold moiety useful for the present disclose, a first scaffold moiety, a second scaffold moiety, and/or a third scaffold moiety, includes a conventional exosome protein, including, but not limiting, tetraspanin molecules (e.g., CD63, CD81, CD9 and others), lysosome-associated membrane protein 2 (LAMP2 and LAMP2B), platelet-derived growth factor receptor (PDGFR), GPI anchor proteins, lactadherin and fragments thereof, peptides that have affinity to any of these proteins or fragments thereof, or any combination thereof.

In some aspects, the surface (e.g., Scaffold X)-engineered EVs described herein demonstrate superior characteristics compared to EVs known in the art. For example, surface (e.g., Scaffold X)-engineered EVs contain modified proteins more highly enriched on their surface than naturally occurring EVs or the EVs produced using conventional exosome proteins. Moreover, the surface (e.g., Scaffold X)-engineered EVs of the present disclosure can have greater, more specific, or more controlled biological activity compared to naturally occurring EVs or the EVs produced using conventional exosome proteins.

In some aspects the Scaffold X comprises Prostaglandin F2 receptor negative regulator (the PTGFRN polypeptide). The PTGFRN protein can be also referred to as CD9 partner 1 (CD9P-1), Glu-Trp-Ile EWI motif-containing protein F (EWI-F), Prostaglandin F2-alpha receptor regulatory protein, Prostaglandin F2-alpha receptor-associated protein, or CD315. The full length amino acid sequence of the human PTGFRN protein (Uniprot Accession No. Q9P2B2) is shown at Table 2 as SEQ ID NO: 1. The PTGFRN polypeptide contains a signal peptide (amino acids 1 to 25 of SEQ ID NO: 1), the extracellular domain (amino acids 26 to 832 of SEQ ID NO: 1), a transmembrane domain (amino acids 833 to 853 of SEQ ID NO: 1), and a cytoplasmic domain (amino acids 854 to 879 of SEQ ID NO: 1). The mature PTGFRN polypeptide consists of SEQ ID NO: 1 without the signal peptide, i.e., amino acids 26 to 879 of SEQ ID NO: 1. In some aspects, a PTGFRN polypeptide fragment useful for the present disclosure comprises a transmembrane domain of the PTGFRN polypeptide. In other aspects, a PTGFRN polypeptide fragment useful for the present disclosure comprises the transmembrane domain of the PTGFRN polypeptide and (i) at least five, at least 10, at least 15, at least 20, at least 25, at least 30, at least 40, at least 50, at least 70, at least 80, at least 90, at least 100, at least 110, at least 120, at least 130, at least 140, at least 150 amino acids at the N terminus of the transmembrane domain, (ii) at least five, at least 10, at least 15, at least 20, or at least 25 amino acids at the C terminus of the transmembrane domain, or both (i) and (ii).

In some aspects, the fragments of PTGFRN polypeptide lack one or more functional or structural domains, such as IgV.

In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 26 to 879 of SEQ ID NO: 1. In other aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 9. In other aspects, the Scaffold X comprises the amino acid sequence of SEQ ID NO: 9, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the Scaffold X comprises the amino acid sequence of SEQ ID NO: 9 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 9.

In other aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 2, 3, 4, 5, 6, or 7. In other aspects, the Scaffold X comprises the amino acid sequence of SEQ ID NO: 2, 3, 4, 5, 6, or 7, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the Scaffold X comprises the amino acid sequence of SEQ ID NO: 2, 3, 4, 5, 6, or 7 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 2, 3, 4, 5, 6, or 7.

TABLE 2
Exemplary Scaffold X Protein Sequences

| SEQ | | |
|---|---|---|
| ID | | |
| NO. | Protein | Sequence |
| 1. | Full | MGRLASRPLLLALLSLALCRGRVVRVPTATLVRVVGTELVIPCNVSDYDGPSEQNFDWSFSSL |
| | length | GSSFVELASTWEVGFPAQLYQERLQRGEILLRRTANDAVELHIKNVQPSDQGHYKCSTPSTDA |
| | PTGFRN | TVQGNYEDTVQVKVLADSLHVGPSARPPPSLSLREGEPFELRCTAASASPLHTHLALLWEVHR |
| | Protein | GPARRSVLALTHEGRFHPGLGYEQRYHSGDVRLDTVGSDAYRLSVSRALSADQGSYRCIVSEW |
| | | IAEQGNWQEIQEKAVEVATVVIQPSVLRAAVPKNVSVAEGKELDLTCNITTDRADDVRPEVTW |
| | | SFSRMPDSTLPGSRVLARLDRDSLVHSSPHVALSHVDARSYHLLVRDVSKENSGYYYCHVSLW |
| | | APGHNRSWHKVAEAVSSPAGVGVTWLEPDYQVYLNASKVPGFADDPTELACRVVDTKSGEANV |
| | | RFTVSWYYRMNRRSDNVVTSELLAVMDGDWTLKYGERSKQRAQDGDFIFSKEHTDTFNFRIQR |
| | | TTEEDRGNYYCVVSAWTKQRNNSWVKSKDVFSKPVNIFWALEDSVLVVKARQPKPFFAAGNTF |
| | | EMTCKVSSKNIKSPRYSVLIMAEKPVGDLSSPNETKYIISLDQDSVVKLENWTDASRVDGVVL |
| | | EKVQEDEFRYRMYQTQVSDAGLYRCMVTAWSPVRGSLWREAATSLSNPIEIDFQTSGPIFNAS |
| | | VHSDTPSVIRGDLIKLFCIITVEGAALDPDDMAFDVSWFAVHSFGLDKAPVLLSSLDRKGIVT |
| | | TSRRDWKSDLSLERVSVLEFLLQVHGSEDQDFGNYYCSVTPWVKSPTGSWQKEAEIHSKPVFI |
| | | TVKMDVLNAFKYPLLIGVGLSTVIGLLSCLIGYCSSHWCCKKEVQETRRERRRLMSMEMD |
| 2. | PTGFRN | PSARPPPSLS LREGEPFELR CTAASASPLH THLALLWEVH RGPARRSVLA |
| | Protein | LTHEGRFHPG LGYEQRYHSG DVRLDTVGSD AYRLSVSRAL SADQGSYRCI |
| | Fragment | VSEWIAEQGN WQEIQEKAVE VATVVIQPSV LRAAVPKNVS VAEGKELDLT |
| | #1 | CNITTDRADD VRPEVTWSFS RMPDSTLPGS RVLARLDRDS LVHSSPHVAL |
| | | SHVDARSYHL LVRDVSKENS GYYYCHVSLW APGHNRSWHK VAEAVSSPAG |
| | | VGVTWLEPDY QVYLNASKVP GFADDPTELA CRVVDTKSGE ANVRFTVSWY |
| | | YRMNRRSDNV VTSELLAVMD GDWTLKYGER SKQRAQDGDF IFSKEHTDTF |
| | | NFRIQRTTEE DRGNYYCVVS AWTKQRNNSW VKSKDVFSKP VNIFWALEDS |
| | | VLVVKARQPK PFFAAGNTFE MTCKVSSKNI KSPRYSVLIM AEKPVGDLSS |
| | | PNETKYIISL DQDSVVKLEN WTDASRVDGV VLEKVQEDEF RYRMYQTQVS |
| | | DAGLYRCMVT AWSPVRGSLW REAATSLSNP IEIDFQTSGP IFNASVHSDT |
| | | PSVIRGDLIK LFCIITVEGA ALDPDDMAFD VSWFAVHSFG LDKAPVLLSS |
| | | LDRKGIVTTS RRDWKSDLSL ERVSVLEFLL QVHGSEDQDF GNYYCSVTPW |
| | | VKSPTGSWQK EAEIHSKPVF ITVKMDVLNA FKYPLLIGVG LSTVIGLLSC |
| | | LIGYCSSHWC CKKEVQETRR ERRRLMSMEM D |
| 3. | PTGFRN | VATVVIQPSV LRAAVPKNVS VAEGKELDLT CNITTDRADD VRPEVTWSFS |
| | Protein | RMPDSTLPGS RVLARLDRDS LVHSSPHVAL SHVDARSYHL LVRDVSKENS |
| | Fragment | GYYYCHVSLW APGHNRSWHK VAEAVSSPAG VGVTWLEPDY QVYLNASKVP |
| | #2 | GFADDPTELA CRVVDTKSGE ANVRFTVSWY YRMNRRSDNV VTSELLAVMD |
| | | GDWTLKYGER SKQRAQDGDF IFSKEHTDTF NFRIQRTTEE DRGNYYCVVS |
| | | AWTKQRNNSW VKSKDVFSKP VNIFWALEDS VLVVKARQPK PFFAAGNTFE |
| | | MTCKVSSKNI KSPRYSVLIM AEKPVGDLSS PNETKYIISL DQDSVVKLEN |
| | | WTDASRVDGV VLEKVQEDEF RYRMYQTQVS DAGLYRCMVT AWSPVRGSLW |
| | | REAATSLSNP IEIDFQTSGP IFNASVHSDT PSVIRGDLIK LFCIITVEGA |
| | | ALDPDDMAFD VSWFAVHSFG LDKAPVLLSS LDRKGIVTTS RRDWKSDLSL |
| | | ERVSVLEFLL QVHGSEDQDF GNYYCSVTPW VKSPTGSWQK EAEIHSKPVF |
| | | ITVKMDVLNA FKYPLLIGVG LSTVIGLLSC LIGYCSSHWC CKKEVQETRR |
| | | ERRRLMSMEM D |
| 4. | PTGFRN | SPAGVGVTWL EPDYQVYLNA SKVPGFADDP TELACRVVDT KSGEANVRFT |
| | Protein | VSWYYRMNRR SDNVVTSELL AVMDGDWTLK YGERSKQRAQ DGDFIFSKEH |
| | Fragment | TDTFNFRIQR TTEEDRGNYY CVVSAWTKQR NNSWVKSKDV FSKPVNIFWA |
| | #3 | LEDSVLVVKA RQPKPFFAAG NTFEMTCKVS SKNIKSPRYS VLIMAEKPVG |
| | | DLSSPNETKY IISLDQDSVV KLENWTDASR VDGVVLEKVQ EDEFRYRMYQ |
| | | TQVSDAGLYR CMVTAWSPVR GSLWREAATS LSNPIEIDFQ TSGPIFNASV |
| | | HSDTPSVIRG DLIKLFCIIT VEGAALDPDD MAFDVSWFAV HSFGLDKAPV |
| | | LLSSLDRKGI VTTSRRDWKS DLSLERVSVL EFLLQVHGSE DQDFGNYYCS |
| | | VTPWVKSPTG SWQKEAEIHS KPVFITVKMD VLNAFKYPLL IGVGLSTVIG |
| | | LLSCLIGYCS SHWCCKKEVQ ETRRERRRLM SMEMD |
| 5. | PTGFRN | KPVNIFWALE DSVLVVKARQ PKPFFAAGNT FEMTCKVSSK NIKSPRYSVL |
| | Protein | IMAEKPVGDL SSPNETKYII SLDQDSVVKL ENWTDASRVD GVVLEKVQED |
| | Fragment | EFRYRMYQTQ VSDAGLYRCM VTAWSPVRGS LWREAATSLS NPIEIDFQTS |
| | #4 | GPIFNASVHS DTPSVIRGDL IKLFCIITVE GAALDPDDMA FDVSWFAVHS |
| | | FGLDKAPVLL SSLDRKGIVT TSRRDWKSDL SLERVSVLEF LLQVHGSEDQ |
| | | DFGNYYCSVT PWVKSPTGSW QKEAEIHSKP VFITVKMDVL NAFKYPLLIG |
| | | VGLSTVIGLL SCLIGYCSSH WCCKKEVQET RRERRRLMSM EMD |
| 6. | PTGFRN | VRGSLWREAA TSLSNPIEID FQTSGPIFNA SVHSDTPSVI RGDLIKLFCI |
| | Protein | ITVEGAALDP DDMAFDVSWF AVHSFGLDKA PVLLSSLDRK GIVTTSRRDW |
| | Fragment | KSDLSLERVS VLEFLLQVHG SEDQDFGNYY CSVTPWVKSP TGSWQKEAEI |
| | #5 | HSKPVFITVK MDVLNAFKYP LLIGVGLSTV IGLLSCLIGY CSSHWCCKKE |
| | | VQETRRERRR LMSMEMD |
| 7. | PTGFRN | SKPVFITVKM DVLNAFKYPL LIGVGLSTVI GLLSCLIGYC SSHWCCKKEV |
| | Protein | QETRRERRRL MSMEMD |
| | Fragment | |
| | #6 | |
| 8. | PTGFRN | MGRLASRPLL LALLSLALCR |
| | Protein- | |
| | Signal | |
| | Peptide | |
| 9. | PTGFRN | GPIFNASVHS DTPSVIRGDL IKLFCIITVE GAALDPDDMA FDVSWFAVHS |
| | Protein | FGLDKAPVLL SSLDRKGIVT TSRRDWKSDL SLERVSVLEF LLQVHGSEDQ |
| | Fragment | DFGNYYCSVT PWVKSPTGSW QKEAEIHSKP VFITVKMDVL NAFKYPLLIG |
| | #7 | VGLSTVIGLL SCLIGYCSSH WCCKKEVQET RRERRRLMSM EMD |

Non-limiting examples of other Scaffold X proteins can be found at U.S. Pat. No. 10,195,290 B1 and 10,561,740 B2, each of which is incorporated by reference in its entirety.

In some aspects, the scaffold moiety expressed in an EV is not fused to the payload, e.g., a biologically active moiety, e.g., a STING agonist.

In some aspects, Scaffold X can be used to link any moiety to the luminal surface and on the exterior surface of the EV, e.g., exosome, at the same time. For example, the PTGFRN polypeptide can be used to link an antigen, an adjuvant, and/or an immune modulator inside the lumen (e.g., on the luminal surface) in addition to the exterior surface of the EV, e.g., exosome. Therefore, in certain aspects, Scaffold X can be used for dual purposes, e.g., an antigen on the luminal surface and an adjuvant or immune modulator on the exterior surface of the EV, e.g., exosome, an antigen on the exterior surface of the EV, e.g., exosome, and the adjuvant or immune modulator on the luminal surface, an adjuvant on the luminal surface and an immune modulator on the exterior surface of the EV, e.g., exosome, or an immune modulator on the luminal surface and an adjuvant on the exterior surface of the EV, e.g., exosome.

In some aspects, the scaffold protein comprises a Scaffold Y. Non-limiting examples of Scaffold Y proteins that can be used in the compositions and methods disclosed herein include those Scaffold Y proteins disclosed, for example, in International Publication No. WO/2019/099942 or WO 2020/101740, each of which is incorporated herein by reference in its entirety. In some aspects, the Scaffold Y protein is selected from myristoylated alanine rich Protein Kinase C substrate (“the MARCKS protein”); myristoylated alanine rich Protein Kinase C substrate like 1 (“the MARCKSL1 protein”); brain acid soluble protein 1 (“the BASP1 protein”). In some aspects, a Scaffold Y protein can be a whole protein or a fragment thereof (e.g., functional fragment, e.g., the smallest fragment that is capable of anchoring a moiety on the luminal surface of the EVs, e.g., exosomes). In some aspects, a Scaffold Y can anchor a moiety (e.g., a STING agonist and/or an IL-12 moiety) to the lumen of the EVs, e.g., exosomes.

IIIC. Anchoring Moieties

In some aspects, one or more payloads can be linked to an anchoring moiety. In some aspects, anchoring moieties that can be used to link a payload to the exterior surface and/or luminal surface of the EV (e.g., exosome) comprises: a sterol (e.g., cholesterol), GM1, a lipid (e.g., fatty acid), a vitamin, a small molecule, a peptide, or a combination thereof.

In some aspects, the anchoring moiety is a lipid. A lipid anchoring moiety can be any lipid known in the art, e.g., palmitic acid or glycosylphosphatidylinositols. In some aspects, the lipid is a fatty acid, phosphatide, phospholipid (e.g., phosphatidyl choline, phosphatidyl serine, or phosphatidyl ethanolamine), or analogue thereof (e.g. phophatidylcholine, lecithin, phosphatidylethanolamine, cephalin, or phosphatidylserine or analogue or portion thereof, such as a partially hydrolyzed portion thereof).

Generally, anchoring moieties are chemically attached. However, an anchoring moiety can be attached to a payload enzymatically.

Some types of membrane anchors that can be used to practice the methods of the present disclosure are presented in the following table:

| Modification | Modifying Group |
|---|---|
| S-Palmitoylation | 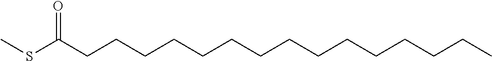 [SMILES: CCCCCCCCCCCCCCCC(=O)SC] |
| N-Palmitoylation | 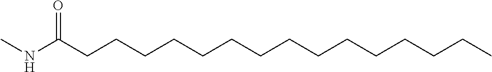 [SMILES: CCCCCCCCCCCCCCCC(=O)NC] |
| N-Myristoylation | 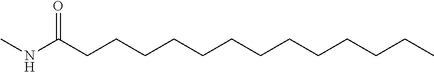 [SMILES: CCCCCCCCCCCCCC(=O)NC] |
| O-Acylation | 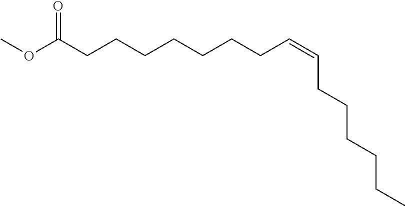 [SMILES: CCCCCC/C=C\CCCCCCCC(=O)OC] |
| Farnesylation | 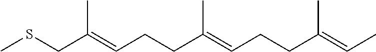 [SMILES: C/C=C(\C)CC/C=C(\C)CC/C=C(\C)CSC] |
| Geranylgeranylation | 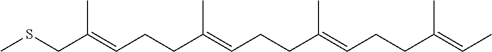 [SMILES: C/C=C(\C)CC/C=C(\C)CC/C=C(\C)CC/C=C(\C)CSC] |
| Cholesterol | 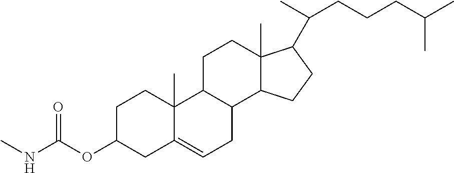 [SMILES: CNC(=O)OC1CCC2(C)C(=CCC3C2CCC2(C)C(C(C)CCCC(C)C)CCC32)C1] |

In some aspects, an anchoring moiety of the present disclosure can comprise two or more types of anchoring moieties disclosed herein. For example, in some aspects, an anchoring moiety can comprise two lipids, e.g., a phospholipids and a fatty acid, or two phospholipids, or two fatty acids, or a lipid and a vitamin, or cholesterol and a vitamin.

In some aspects, the anchoring moiety useful for the present disclosure comprises a sterol, steroid, hopanoid, hydroxysteroid, secosteroid, or analog thereof with lipophilic properties. In some aspects, the anchoring moiety comprises a sterol, such as a phytosterol, mycosterol, or zoosterol. Exemplary zoosterols include cholesterol and 24S-hydroxycholesterol; exemplary phytosterols include ergosterol (mycosterol), campesterol, sitosterol, and stigmasterol. In some aspects, the sterol is selected from ergosterol, 7-dehydrocholesterol, cholesterol, 24S-hydroxycholesterol, lanosterol, cycloartenol, fucosterol, saringosterol, campesterol, β-sitosterol, sitostanol, coprostanol, avenasterol, or stigmasterol. Sterols may be found either as free sterols, acylated (sterol esters), alkylated (steryl alkyl ethers), sulfated (sterol sulfate), or linked to a glycoside moiety (steryl glycosides), which can be itself acylated (acylated sterol glycosides). In some aspects, the anchoring moiety is a cholesterol.

In some aspects, the anchoring moiety comprises a steroid. In some aspects, the steroid is selected from dihydrotestosterone, uvaol, hecigenin, diosgenin, progesterone, or cortisol.

In some aspects, the anchoring moiety is a fatty acid. In some aspects, the fatty acid is a short-chain, medium-chain, or long-chain fatty acid. In some aspects, the fatty acid is a saturated fatty acid. In some aspects, the fatty acid is an unsaturated fatty acid. In some aspects, the fatty acid is a monounsaturated fatty acid. In some aspects, the fatty acid is a polyunsaturated fatty acid, such as an omega-3 or omega-6 fatty acid.

In some aspects, the anchoring moiety comprises a phospholipid. Phospholipids are a class of lipids that are a major component of all cell membranes. They can form lipid bilayers because of their amphiphilic characteristic. The structure of the phospholipid molecule generally consists of two hydrophobic fatty acid “tails” and a hydrophilic “head” consisting of a phosphate group. For example, a phospholipid can be a lipid according to the following formula:

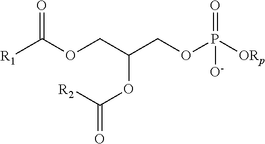

in which Rₚ represents a phospholipid moiety and R₁ and R₂ represent fatty acid moieties with or without unsaturation that may be the same or different.

In some aspects, a payload is linked to an anchoring moiety disclosed herein via a linker combination, which can comprise any combination of cleavable and/or non-cleavable linkers. Not to be bound by any one theory, one of the functions of a linker combination is to provide the optimal spacing between the anchoring moiety and the payload.

IIID. Linkers

As described supra, extracellular vesicles (EVs) of the present disclosure (e.g., exosomes and nanovesicles) can comprises one or more linkers that link one or more exogenous biologically active moieties disclosed herein to the EVs (e.g., to the exterior surface or on the luminal surface). In some aspects, the one or more exogenous biologically active moieties are linked to the EVs directly or via one or more scaffold moieties (e.g., Scaffold X). For example, in certain aspects, one or more exogenous biologically active moieties are linked to the exterior surface of an exosome via Scaffold X. In further aspects, one or more exogenous biologically active moieties are linked to the luminal surface of an exosome via Scaffold X. The linker can be any chemical moiety known in the art.

As used herein, the term “linker” refers to a peptide or polypeptide sequence (e.g., a synthetic peptide or polypeptide sequence) or to a non-polypeptide, e.g., an alkyl chain. In some aspects, two or more linkers can be linked in tandem. When multiple linkers are present, each of the linkers can be the same or different. Generally, linkers provide flexibility or prevent/ameliorate steric hindrances. Linkers are not typically cleaved; however in certain aspects, such cleavage can be desirable. Accordingly, in some aspects, a linker can comprise one or more protease-cleavable sites, which can be located within the sequence of the linker or flanking the linker at either end of the linker sequence.

In some aspects, the linker is a peptide linker. In some aspects, the peptide linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids.

In some aspects, the peptide linker is synthetic, i.e., non-naturally occurring. In one aspect, a peptide linker includes peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in one aspect the peptide linker can comprise non-naturally occurring polypeptides which are modified forms of naturally occurring polypeptides (e.g., comprising a mutation such as an addition, substitution or deletion).

Linkers can be susceptible to cleavage (“cleavable linker”) thereby facilitating release of the exogenous biologically active moiety.

In some aspects, the linker is a “reduction-sensitive linker.” In some aspects, the reduction-sensitive linker contains a disulfide bond. In some aspects, the linker is an “acid labile linker.” In some aspects, the acid labile linker contains hydrazone. Suitable acid labile linkers also include, for example, a cis-aconitic linker, a hydrazide linker, a thiocarbamoyl linker, or any combination thereof.

In some aspects, the ASO is associated with the EV, e.g., exosome, by way of a linker. In some aspects, the linker comprises acrylic phosphoramidite (e.g., ACRYDITE™) adenylation, azide (NHS Ester), digoxigenin (NHS Ester), cholesterol-TEG, I-LINKER™, an amino modifier (e.g., amino modifier C6, amino modifier C12, amino modifier C6 dT, or Uni-Link™ amino modifier), alkyne, 5′ Hexynyl, 5-Octadiynyl dU, biotinylation (e.g., biotin, biotin (Azide), biotin dT, biotin-TEG, dual biotin, PC biotin, or desthiobiotin), thiol modification (thiol modifier C3 S-S, dithiol or thiol modifier C6 S-S), or any combination thereof.

In some aspects, the linker comprises a terpene such as nerolidol, farnesol, limonene, linalool, geraniol, carvone, fenchone, or menthol; a lipid such as palmitic acid or myristic acid; cholesterol; oleyl; retinyl; cholesteryl residues; cholic acid; adamantane acetic acid; 1-pyrene butyric acid; dihydrotestosterone; 1,3-Bis-O(hexadecyl)glycerol; geranyloxyhexyl group; hexadecylglycerol; borneol; 1,3-propanediol; heptadecyl group; 03-(oleoyl)lithocholic acid; 03-(oleoyl)cholenic acid; dimethoxytrityl; phenoxazine, a maleimide moiety, a glucorinidase type, a CL2A-SN38 type, folic acid; a carbohydrate; vitamin A; vitamin E; vitamin K, or any combination thereof. In certain aspects, the ASO comprises a cholesterol tag, and the cholesterol tag associates with the membrane of the EV, e.g., exosome. In some aspects, the linker comprises a non-cleavable linker.

In some aspects, the linker comprises tetraethylene glycol (TEG), hexaethylene glycol (HEG), polyethylene glycol (PEG), succinimide, or any combination thereof. In some aspects, the linker comprises a spacer unit to link the biologically active molecule to the linker.

In some aspects, one or more linkers comprise smaller units (e.g., HEG, TEG, glycerol, C2 to C12 alkyl, and the like) linked together. In one aspect, the linkage is an ester linkage (e.g., phosphodiester or phosphorothioate ester) or other linkage.

In some aspects, the linker comprises a polyethylene glycol (PEG) characterized by a formula R³—(O—CH₂—CH₂)ₙ— or R³—(O—CH₂—CH₂)ₙ—O— with R³ being hydrogen, methyl or ethyl and n having a value from 2 to 200. In some aspects, the linker comprises a spacer, wherein the spacer is PEG.

In some aspects, the PEG linker is an oligo-ethylene glycol, e.g., diethylene glycol, triethylene glycol, tetra ethylene glycol (TEG), pentaethylene glycol, or a hexaethylene glycol (HEG) linker.

### IV. Methods of Treatment

Administration of the presently disclosed pharmaceutical compositions for treating a plurality of diseases or conditions where administration of EVs are of beneficial effect to a subject, is further contemplated. In some aspects, the methods of treating a disease or a condition in a subject disclosed herein comprise administering to the subject the pharmaceutical composition.

In some aspects, the present disclosure provides a composition which can be administered by a parenteral, topical, intravenous, oral, subcutaneous, intra-arterial, intradermal, transdermal, rectal, intracranial, intraperitoneal, intranasal, intratumoral, intramuscular route, or as an inhalant. In some aspects, the pharmaceutical composition comprising EVs is administered intravenously, e.g. by injection. For transmucosal or transdermal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of nasal sprays or suppositories. For transdermal administration, the modified exosomes are formulated into ointments, salves, gels, or creams as generally known in the art.

In some aspects, the EVs are administered intravenously to the circulatory system of the subject. In some aspects, the EVs are infused in suitable liquid and administered into a vein of the subject. In some aspects, the EVs are administered intra-arterialy to the circulatory system of the subject. In some aspects, the EVs are infused in suitable liquid and administered into an artery of the subject. In some aspects, the EVs are administered to the subject by intrathecal administration. In some aspects, the EVs are administered via an injection into the spinal canal, or into the subarachnoid space so that it reaches the cerebrospinal fluid (CSF). In some aspects, the EVs are administered intratumorally into one or more tumors of the subject. In some aspects, the EVs are administered to the subject by intranasal administration. In some aspects, the EVs can be insufflated through the nose in a form of either topical administration or systemic administration. In certain aspects, the EVs are administered as nasal spray.

In some aspects, the EVs are administered to the subject by intraperitoneal administration. In some aspects, the EVs are infused in suitable liquid and injected into the peritoneum of the subject. In some aspects, the intraperitoneal administration results in distribution of the EVs to the lymphatics. In some aspects, the intraperitoneal administration results in distribution of the EVs to the thymus, spleen, and/or bone marrow. In some aspects, the intraperitoneal administration results in distribution of the EVs to one or more lymph nodes. In some aspects, the intraperitoneal administration results in distribution of the EVs to one or more of the cervical lymph node, the inguinal lymph node, the mediastinal lymph node, or the sternal lymph node. In some aspects, the intraperitoneal administration results in distribution of the EVs to the pancreas.

In some aspects, the EVs, e.g., exosomes, are administered to the subject by periocular administration. In some aspects, the s are injected into the periocular tissues. Periocular drug administration includes the routes of subconjunctival, anterior sub-Tenon's, posterior sub-Tenon's, and retrobulbar administration.

In some aspects, the treatment is prophylactic. In some aspects, the EVs for the present disclosure are used to induce an immune response. In some aspects, the EVs for the present disclosure are used to vaccinate a subject.

In some aspects, the disease or condition is a cancer, a fibrosis, a hemophilia, diabetes, a growth factor deficiency, an eye disease, a Pompe disease, a lysosomal storage disorder, mucovicidosis, cystic fibrosis, Duchenne and Becker muscular dystrophy, transthyretin amyloidosis, hemophilia A, hemophilia B, adenosine-deaminase deficiency, Leber's congenital amaurosis, X-linked adrenoleukodystrophy, metachromatic leukodystrophy, OTC deficiency, glycogen storage disease 1A, Criggler-Najjar syndrome, primary hyperoxaluria type 1, acute intermittent porphyria, phenylketonuria, familial hypercholesterolemia, mucopolysaccharidosis type VI, α1 antitrypsin deficiency, and a hypercholesterolemia.

In some aspects, the disease or disorder is a graft-versus-host disease (GvHD). In some aspects, the disease or disorder that can be treated with the present disclosure is an autoimmune disease. Non-limiting examples of autoimmune diseases include: multiple sclerosis, peripheral neuritis, Sjogren's syndrome, rheumatoid arthritis, alopecia, autoimmune pancreatitis, Behcet's disease, Bullous pemphigoid, Celiac disease, Devic's disease (neuromyelitis optica), Glomerulonephritis, IgA nephropathy, assorted vasculitides, scleroderma, diabetes, arteritis, vitiligo, ulcerative colitis, irritable bowel syndrome, psoriasis, uveitis, systemic lupus erythematosus, and combinations thereof.

In some aspects, the disease or disorder is an infectious disease. In certain aspects, the disease or disorder is an oncogenic virus. In some aspects, infectious diseases that can be treated with the present disclosure includes, but not limited to, Human Gamma herpes virus 4 (Epstein Barr virus), influenza A virus, influenza B virus, cytomegalovirus, Staphylococcus aureus, Mycobacterium tuberculosis, Chlamydia trachomatis, HIV-1, HIV-2, corona viruses (e.g., MERS-CoV and SARS CoV), filoviruses (e.g., Marburg and Ebola), Streptococcus pyogenes, Streptococcus pneumoniae, Plasmodia species (e.g., vivax and falciparum), Chikungunya virus, Human Papilloma virus (HPV), Hepatitis B, Hepatitis C, human herpes virus 8, herpes simplex virus 2 (HSV2), Klebsiella sp., Pseudomonas aeruginosa, Enterococcus sp., Proteus sp., Enterobacter sp., Actinobacter sp., coagulase-negative staphylococci (CoNS), Mycoplasma sp., or combinations thereof.

In some aspects, the cancer is bladder cancer, cervical cancer, renal cell cancer, testicular cancer, colorectal cancer, lung cancer, head and neck cancer, ovarian, lymphoma, liver cancer, glioblastoma, melanoma, myeloma, leukemia, pancreatic cancer, or combinations thereof.

In certain aspects, the cancer is associated with increased expression of a STAT6 protein. Non-limiting examples of cancers that can be treated with the present disclosure include a colorectal cancer, lung cancer (e.g., non-small cell lung cancer (NSCLC)), pancreatic cancer (e.g., pancreatic ductal adenocarcinoma (PDAC)), leukemia, uterine cancer, ovarian cancer, bladder cancer, bile duct cancer, gastric cancer, or any combination thereof. In some aspects, the cancer is selected from colon adenocarcinoma, rectum adenocarcinoma, pancreatic adenocarcinoma, pancreatic ductal adenocarcinoma (PDAC), ovarian serous cystadenocarcinoma, acute myelid leukemia, testicular cancer (e.g., testicular germ cell tumors, seminoma, non-seminoma and choriocarcinoma), lung adenocarcinoma, brain lower grade glioma, glioblastoma multiforme, uveal melanoma, thyroid carcinoma, uterine corpus endometrial carcinoma, uterine carcinosarcoma, pheochromocytoma, paraganglioma, and any combination thereof. In certain aspects, the cancer is a myeloid-rich cancer. In some aspects, the cancer comprises a liver cancer. In some aspects, the cancer comprises hepatocellular cancer (HCC). In some aspects, the cancer comprises pancreatic ductal adenocarcinoma (PDAC), in some aspects, the cancer comprises colorectal carcinoma (CRC). In some aspects, the cancer comprises ovarian cancer. In some aspects, the cancer comprises leptomeningeal cancer.

When administered to a subject with a cancer, in certain aspects, EVs of the present disclosure can up-regulate an immune response and enhance the tumor targeting of the subject's immune system. In some aspects, the cancer being treated is characterized by infiltration of leukocytes (T-cells, B-cells, macrophages, dendritic cells, monocytes) into the tumor microenvironment, or so-called “hot tumors” or “inflammatory tumors”. In some aspects, the cancer being treated is characterized by low levels or undetectable levels of leukocyte infiltration into the tumor microenvironment, or so-called “cold tumors” or “non-inflammatory tumors”. In some aspects, an EV is administered in an amount and for a time sufficient to convert a “cold tumor” into a “hot tumor”, i.e., said administering results in the infiltration of leukocytes (such as T-cells) into the tumor microenvironment. In certain aspects, cancer comprises bladder cancer, cervical cancer, renal cell cancer, testicular cancer, colorectal cancer, lung cancer, head and neck cancer, and ovarian, lymphoma, liver cancer, glioblastoma, melanoma, myeloma, leukemia, pancreatic cancers, or combinations thereof. In other term, “distal tumor” or “distant tumor” refers to a tumor that has spread from the original (or primary) tumor to distant organs or distant tissues, e.g., lymph nodes. In some aspects, the EVs of the disclosure treats a tumor after the metastatic spread.

V. Methods of Producing Engineered Exosomes

EVs, e.g., exosomes, of the present disclosure can be produced from a cell grown in vitro or a body fluid of a subject. When exosomes are produced from in vitro cell culture, various producer cells can be used.

In some aspects, the producer cell can be a mammalian cell line, a plant cell line, an insect cell line, a fungi cell line, or a prokaryotic cell line. In certain aspects, the producer cell is a mammalian cell line. Non-limiting examples of mammalian cell lines include: a human embryonic kidney (HEK) cell line, a Chinese hamster ovary (CHO) cell line, an HT-1080 cell line, a HeLa cell line, a PERC-6 cell line, a CEVEC cell line, a fibroblast cell line, an amniocyte cell line, an epithelial cell line, a mesenchymal stem cell (MSC) cell line, and combinations thereof. In certain aspects, the mammalian cell line comprises HEK-293 cells, BJ human foreskin fibroblast cells, fHDF fibroblast cells, AGE.HN® neuronal precursor cells, CAP® amniocyte cells, adipose mesenchymal stem cells, RPTEC/TERT1 cells, or combinations thereof. In some aspects, the producer cell is a primary cell. In certain aspects, the primary cell can be a primary mammalian cell, a primary plant cell, a primary insect cell, a primary fungi cell, or a primary prokaryotic cell.

In some aspects, the producer cell is not an immune cell, such an antigen presenting cell, a T cell, a B cell, a natural killer cell (NK cell), a macrophage, a T helper cell, or a regulatory T cell (Treg cell). In other aspects, the producer cell is not an antigen presenting cell (e.g., dendritic cells, macrophages, B cells, mast cells, neutrophils, Kupffer-Browicz cell, or a cell derived from any such cells).

The producer cell can be genetically modified to comprise one or more exogenous sequences (e.g., encoding one or more exogenous biologically active moieties disclosed herein, e.g., an immune modulator, e.g., IL-12, or an ASO, e.g., STAT6 ASO) to produce exosomes described herein. The genetically-modified producer cell can contain the exogenous sequences by transient or stable transformation. The exogenous sequences can be transformed as a plasmid. The exogenous sequences can be stably integrated into a genomic sequence of the producer cell, at a targeted site or in a random site. In some aspects, a stable cell line is generated for production of EVs disclosed herein, e.g., exosomes.

The exogenous sequences can be inserted into a genomic sequence of the producer cell, located within, upstream (5′-end) or downstream (3′-end) of an endogenous sequence encoding an exosome protein. Various methods known in the art can be used for the introduction of the exogenous sequences into the producer cell. For example, cells modified using various gene editing methods (e.g., methods using a homologous recombination, transposon-mediated system, loxP-Cre system, CRISPR/Cas9 or TALEN) are within the scope of the present disclosure.

The exogenous sequences can comprise a sequence encoding a scaffold moiety disclosed herein or a fragment or variant thereof. An extra copy of the sequence encoding a scaffold moiety can be introduced to produce an exosome described herein (e.g., having a higher density of a scaffold moiety or expressing multiple different scaffold moieties on the surface or on the luminal surface of the EV, e.g., exosome). Exogenous sequences encoding a modification or a fragment of a scaffold moiety can be introduced to produce a lumen-engineered and/or surface-engineered exosome containing the modification or the fragment of the scaffold moiety.

In some aspects, a producer cell can be modified, e.g., transfected, with one or more vectors encoding one or more scaffold moieties linked to exogenous biologically active moieties described herein.

VI. Methods of Producing EVs with Payload

VIA. Methods for Loading Paylod

Payloads, e.g., biologically active moieties, e.g., STING agonists, can be encapsulated in EVs, e.g., exosomes, via any appropriate technique known in the art. It is contemplated that all known manners of loading biomolecules into EVs, e.g., exosomes, are deemed suitable for use herein. Such techniques include passive diffusion, electroporation, chemical or polymeric transfection, viral transduction, mechanical membrane disruption or mechanical shear, or any combination thereof. The payloads, e.g., biologically active moieties, e.g., STING agonists, and an EV, e.g., exosome, can be incubated in an appropriate buffer during encapsulation.

In one aspect, a biologically active moiety, e.g., a STING agonist, is encapsulated by an EV, e.g., exosome, by passive diffusion. The biologically active moiety, e.g., a STING agonist, and the EV, e.g., exosome, can be mixed together and incubated for a time period sufficient for the biologically active moiety, e.g., STING agonist, to diffuse through the vesicle lipid bilayer, thereby becoming encapsulated in the EV, e.g., exosome. The biologically active moiety, e.g., a STING agonist, and the EV, e.g., exosome, can be incubated together for between about 1 to 30 hours, 2 to 24 hours, 4 to 18 hours, 6 to 16 hours, 8 to 14 hours, 10 to 12 hours, 6 to 12 hours, 12 to 20 hours, 14 to 18 hours, or 20 to 30 hours. The STING agonist and the EV, e.g., exosome, can be incubated together for about 2 hours, 4 hours, 6, hours, 8, hours, 10, hours, 12 hours, 14 hours, 16 hours, 18 hours, 20 hours, 22 hours, 24 hours, 26 hours, or 30 hours.

The buffer conditions of the solution of EVs, e.g., exosomes, can also be altered to increase or control encapsulation of the biologically active moiety, e.g., STING agonist. In one aspect, the buffer can be a phosphate buffered saline (PBS) with sucrose. Additional buffer modifications can also be used, such as shear protectants, viscosity modifiers, and/or solutes that affect vesicle structural properties. Excipients can also be added to improve the efficiency of the biologically active moiety, e.g., STING agonist, encapsulation such as membrane softening materials and molecular crowding agents. Other modifications to the buffer can include specific pH ranges and/or concentrations of salts, organic solvents, small molecules, detergents, zwitterions, amino acids, polymers, and/or any combination of the above including multiple concentrations.

The temperature of the solution of EVs, e.g., exosomes, and the biologically active moieties, e.g., STING agonists, during incubation can be changed to increase or control encapsulation of the biologically active moieties. The temperature can be room temperature. The temperature can be between about 15° C. to 900 C, 15-30° C., 30-50° C., 50-90° C. In some aspects, the temperature is about 150 C, 20° C., 350 C, 30° C., 350 C, 37° C., 400 C, 45° C., 500 C, 55° C., 600 C, 65° C., 70° C., 75° C., 800 C, 85° C., or 90° C.

The concentration of the biologically active moiety, e.g., STING agonist, during the incubation of the biologically active moiety with the EVs, e.g., exosomes, can also be altered to increase or control encapsulation of the biologically active moiety, e.g., STING agonist. For example, the concentration of a biologically active moiety, e.g., a STING agonist, can be between at least 0.01 mM and 100 mM STING agonist. The concentration of a biologically active moiety, e.g., a STING agonist, can be at least 0.01-1 mM, 1-10 mM, 10-50 mM, or 50-100 mM. The concentration of the agonist can be at least 0.01 mM, 0.02 mM, 0.03 mM, 0.04 mM, 0.05 mM, 0.06 mM, 0.07 mM, 0.08 mM, 0.09 mM, 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 15 mM, 20 mM 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. In some aspects, the concentration of a biologically active moiety, e.g., a STING agonist, is at least about 1 mM.

In some aspects, biologically active moieties, e.g., STING agonists, are incubated in the mixture at a concentration of at least about 500 μM, at least about 600 μM, at least about 700 μM, at least about 800 μM, at least about 900 μM, at least about 1000 μM, at least about 1100 μM, at least about 1200 μM, at least about 1300 μM, at least about 1400 μM, at least about 1500 μM, at least about 1600 μM, at least about 1700 μM, at least about 1800 μM, at least about 1900 μM, or at least about 2000 μM. In some aspects, the CDNs are incubated in the mixture at a concentration of at least about 2500 μM, at least about 2600 μM, at least about 2700 μM, at least about 2800 μM, at least about 2900 μM, at least about 3000 μM, at least about 3100 μM, at least about 3200 μM, at least about 3300 μM, at least about 3400 μM, at least about 3500 μM, at least about 3600 μM, at least about 3700 μM, at least about 3800 μM, at least about 3900 μM, or at least about 3000 μM.

In some aspects, the biologically active moieties, e.g., STING agonists, are incubated in the mixture at a concentration of between about 500 μM and about 100 mM, between about 500 μM and about 90 mM, between about 500 μM and about 80 mM, between about 500 μM and about 70 mM, between about 500 μM and about 60 mM, between about 500 μM and about 50 mM, between about 500 μM and about 40 mM, between about 500 μM and about 30 mM, between about 500 μM and about 20 mM, between about 500 μM and about 10 mM, or between about 500 μM and about 1 mM. In some aspects, the biologically active moieties, e.g., STING agonists, are incubated in the mixture between about 500 μM and about 10 mM, between about 500 μM and about 9 mM, between about 500 μM and about 8 mM, between about 500 μM and about 7 mM, between about 500 μM and about 6 mM, between about 500 μM and about 5 mM, between about 500 μM and about 4 mM, between about 500 μM and about 3 mM, between about 500 μM and about 2 mM, or between about 500 μM and about 1 mM.

The number of extracellular particles incubated with the biologically active moieties, e.g., STING agonists, can also be altered to increase or control encapsulation of the biologically active moieties, e.g., STING agonists. The number of purified EV, e.g., exosome, particles can be between at least about 10⁶ to at least about 10²⁰ total particles of purified vesicles. The number of purified particles can be between about 10⁸ to 10¹8, 10¹⁰ to 10¹⁶, 10⁸ to 10¹⁴, or 10¹⁰ to 10¹² total particles of purified vesicles. The number of purified particles can be at least about 10⁶, 10⁸, 10¹⁰, 10¹², 10¹⁴, 10¹⁶, 1018, or 10²⁰ total particles of purified vesicles.

VIB. Methods for EV Purification

The EVs, e.g., exosomes, prepared for the present disclosure can be isolated from the producer cells. It is contemplated that all known manners of isolation of EVs, e.g., exosomes, are deemed suitable for use herein. For example, physical properties of EVs, e.g., exosomes, can be employed to separate them from a medium or other source material, including separation on the basis of electrical charge (e.g., electrophoretic separation), size (e.g., filtration, molecular sieving, etc), density (e.g., regular or gradient centrifugation), Svedberg constant (e.g., sedimentation with or without external force, etc). Alternatively or additionally, isolation can be based on one or more biological properties, and include methods that can employ surface markers (e.g., for precipitation, reversible binding to solid phase, FACS separation, specific ligand binding, non-specific ligand binding, etc.). In yet further contemplated methods, the EVs, e.g., exosomes, can also be fused using chemical and/or physical methods, including PEG-induced fusion and/or ultrasonic fusion.

Prior to the formulation, the EVs, e.g., exosomes, can also be purified after incubation with the biologically active moieties, e.g., STING agonists, to remove free, unencapsulated biologically active moieties, e.g., STING agonists, from the composition. All manners of previously disclosed methods are also deemed suitable for use herein, including separation on the basis of physical or biological properties of EVs, e.g., exosomes.

Isolation, purification, and enrichment can be done in a general and non-selective manner (typically including serial centrifugation). Alternatively, isolation, purification, and enrichment can be done in a more specific and selective manner (e.g., using producer cell-specific surface markers). For example, specific surface markers can be used in immunoprecipitation, FACS sorting, affinity purification, bead-bound ligands for magnetic separation etc.

In some aspects, size exclusion chromatography can be utilized to isolate or purify the EVs, e.g., exosomes. Size exclusion chromatography techniques are known in the art. Exemplary, non-limiting techniques are provided herein. In some aspects, a void volume fraction is isolated and comprises the EVs, e.g., exosomes, of interest. In some aspects, for example, density gradient centrifugation can be utilized to further isolate the EVs, e.g., exosomes. Still further, in some aspects, it can be desirable to further separate the producer cell-derived EVs, e.g., exosomes, from EVs of other origin. For example, the producer cell-derived EVs, e.g., exosomes, can be separated from non-producer cell-derived EVs, e.g., exosomes, by immunosorbent capture using an antigen antibody specific for the producer cell.

In some aspects, the isolation of EVs, e.g., exosomes, can involve size exclusion chromatography or ion chromatography, such as anion exchange, cation exchange, or mixed mode chromatography. In some aspects, the isolation of EVs, e.g., exosomes, can involve desalting, dialysis, tangential flow filtration, ultrafiltration, or diafiltration, or any combination thereof 0. In some aspects, the isolation of EVs, e.g., exosomes, can involve combinations of methods that include, but are not limited to, differential centrifugation, size-based membrane filtration, concentration and/or rate zonal centrifugation. In some aspects, the isolation of EVs, e.g., exosomes, can involve one or more centrifugation steps. The centrifugation can be performed at about 50,000 to 150,000×g. The centrifugation can be performed at about 50,000×g, 75,000×g, 100,000×g, 125,000×g, or 150,000×g.

The EVs, e.g., exosomes, prepared for the present disclosure can be isolated via a multimodal chromatography. The methods of the present disclosure are useful for preparing a composition comprising extracellular vesicles associated with biologically active moieties, e.g., STING agonists, e.g., one or more cyclic dinucleotides (CDNs, e.g., STING agonists), comprising incubating the extracellular vesicles with the biologically active moieties, e.g., STING agonists, e.g., one or more cyclic dinucleotides (CDNs) in a mixture, and separating the extracellular vesicles using a multimodal chromatography.

The multimodal chromatography can be used to remove free biologically active moieties, e.g., STING agonists, from an EV preparation. It is contemplated that all known manners of isolation of EVs, e.g., exosomes, are deemed suitable for use herein. For example, physical properties of EVs, e.g., exosomes, can be employed to separate them from a medium or other source material, including separation on the basis of electrical charge (e.g., electrophoretic separation), size (e.g., filtration, molecular sieving, etc), density (e.g., regular or gradient centrifugation), Svedberg constant (e.g., sedimentation with or without external force, etc). Alternatively, or additionally, isolation can be based on one or more biological properties, and include methods that can employ surface markers (e.g., for precipitation, reversible binding to solid phase, FACS separation, specific ligand binding, non-specific ligand binding, etc.). In yet further contemplated methods, the EVs, e.g., exosomes, can also be fused using chemical and/or physical methods, including PEG-induced fusion and/or ultrasonic fusion. In some aspects, the multimodal column is selected from a group comprising CaptoCore 700, Capto MMC, or Capto MMC ImpRes. In some aspects, the multimodal column is Captocore 700. In some aspects, the multimodal column is Captocore MMC. In some aspects, the multimodal column is Capto MMC ImpRes.

All of the references cited above, as well as all references cited herein, are incorporated herein by reference in their entireties.

The following examples are offered by way of illustration and not by way of limitation.

### EXAMPLES

The disclosure is further illustrated by the following examples. The examples are provided for illustrative purposes only, and are not to be construed as limiting the scope or content of the disclosure in any way. The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); Green & Sambrook et al., Molecular Cloning: A Laboratory Manual, 4th Edition (Cold Spring Harbor Laboratory Press, 2012); Colowick & Kaplan, Methods In Enzymology (Academic Press); Remington: The Science and Practice of Pharmacy, 22nd Edition (Pharmaceutical Press, 2012); Sundberg & Carey, Advanced Organic Chemistry: Parts A and B, 5th Edition (Springer, 2007).

### Example 1: Evaluation of Stability of Native and Protein X Exosomes

The stability of native exosomes and Protein X-containing exosomes under different pH conditions was evaluated. The evaluation approach chosen to evaluate stability was to monitor size and surface charge via dynamic light scattering and zeta-potential analysis. Materials

Native human embryonic kidney (HEK) cell exosomes were engineered and prepared at approximately 5E12 in MilliQ ultra pure water. Protein X extracellular vesicles (CB-101), were engineered and prepared at approximately 5E12 P/mL in phosphate buffered saline. Gibco phosphate buffered saline pH 7.2 without calcium or magnesium was purchased (PN 20012027, Thermo Fisher Scientific, Waltham, MA). Sterile microcentrifuge tubes (1.5 mL, low retention), were purchased (PN 3451, Thermo Fisher Scientific, Waltham, MA). Additional materials included: citric acid (PN C2404, Sigma-Aldrich, St. Louis, MO), sodium citrate dihydrate (PN BP327, Fisher Scientific Waltham, MA), and carbonate-bicarbonate buffer (PN C3041, Sigma-Aldrich, St. Louis, MO). Phosphate

The two solids (83.01g Na₂HPO₄/2=41.51 g and 12.29g KH₂PO₄/2=6.15 g) were added to a beaker and dissolved in 400 mL of Milli-Q water. The pH was adjusted to 7.4 if necessary with 1M HCl or 1M NaOH. The solution was brought up to 500 mL in a graduated cylinder and filter using a 0.2 m filter flask. In a biosafety cabinet, the solution was transferred into 50 mL centrifuge tubes and store at 4° C. The KH₂PO₄ (Cat #3248-01, Lot #0000163254) and Na₂HPO₄ 7H₂O (Cat #3817-01, Lot #0000201905) were purchased from JT Baker, (Fisher Scientific Waltham, MA).

Dynamic light scattering (DLS)

DLS was used to measure EV aggregation, i.e., DLS is a stability indicating assay. Each sample was prepared and run according to the following method.

Samples were run on a Malvern Zetasizer Nano ZS dynamic light scattering instrument (DLS), model ZEN3600, serial number MAL1036117 (Malvern, Worcestershire, UK) and analyzed with Malvern Zetasizer software version 7.13 (Malvern Panalytical). Malvern DLS micro-cuvettes and cap kit (PN ZEN0040) and zeta potential cuvettes (PN DTS1070) were purchased (Malvern, Worcestershire, UK). Exosomes were diluted from 1E11 P/mL to 1E12 P/mL in Gibco PBS 1×pH 7.2. Samples were measured using low volume Malvern cuvettes (PN ZEN0040), and equilibrated for 4 minutes at 25° C. prior to measuring three times. For each EV sample, 20 μL of sample was diluted into 180 μL of filtered Gibco PBS in a micro-cuvette, mixed by repeat pipetting, and measured.

The following NIST polystyrene size standards from Polysciences Inc. were used in the experiments described herein:

Polysciences Inc. NIST size standards

| Cat# | Lot# | Size (nm) |
|---|---|---|
| 64005 | 704405 | 51.7 ± 0.7 |
| 64010-15 | A758988 | 99.9 ± 1.3 |
| 64014-15 | 712842 | 243.9 ± 0.0 |

### Appearance Testing

Each sample was inspected against a matte white and matte black background for at least 5 seconds against each surface. The tube was gently agitated by hand to stir up potential sediment and inspected for visible particles using a Maglite Mini LED flashlight and a 5x magnifying glass. The pH was measured using Oakton Cole-Parmer pH Spear waterproof pocket pH Tester (Cole-Parmer, Vernon Hills, IL).

### Study Design

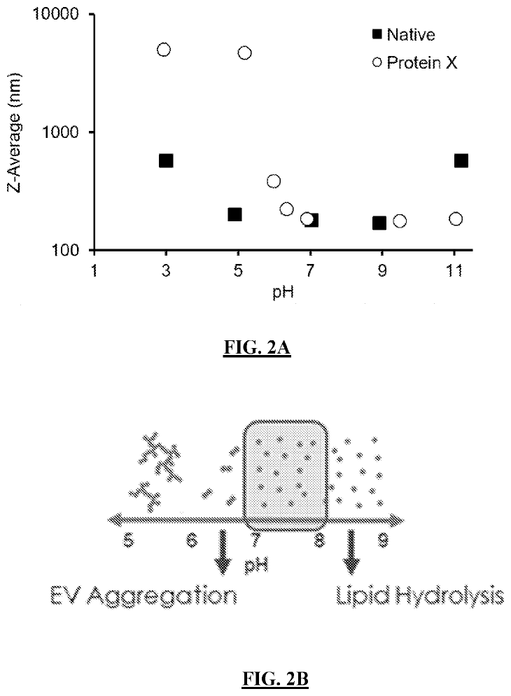

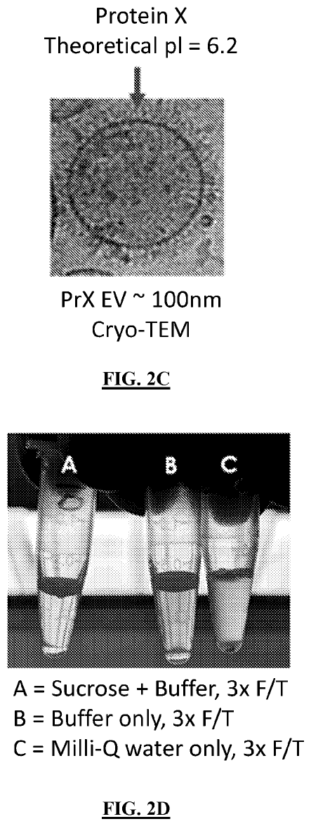

The study design looked at pH values 3, 5, 7, 9, and 11. Measurements were taken immediately following pH adjustments. Concentrated solutions of native and protein X exosomes were prepared in either in MilliQ water, or PBS, and diluted with MilliQ water with the appropriate buffer, and/or adjusted to the correct pH at ambient lab temperature prior to the start of the study (FIG. 2D). For acidic pHs, citrate buffer was used, for neutral pHs, phosphate buffer was used, and for basic pHs, sodium carbonate buffer was used. The target osmolality was 300 mOsm/kg. For the DLS and zeta measurements, 20 μL of EVs at 1E11 to 1E12 P/mL were used. Resulting data for the native and Protein X exosomes are set forth, and in Tables 2, 3, and 4, below.

TABLE 3
Native Exosomes

| | | | | | | | | | Std Dev |
|---|---|---|---|---|---|---|---|---|---|
| | | Sample | T | ZP | Mob | Cond | | Average | (mV) |
| Record | Type | Name | ° C. | mV | μmcm/Vs | mS/cm | pH | (mV) | (mV) |
| 1 | Zeta | pH 3.01 | 25 | 9.05 | 0.7091 | 6.95 | 3.01 | 9.5 | 0.67 |
| | | sample 1 1 | | | | | | | |
| 2 | Zeta | pH 3.01 | 25 | 10 | 0.7848 | 7.25 | | | |
| | | sample 1 2 | | | | | | | |
| 3 | Zeta | pH 4.95 | 25 | −16.5 | −1.291 | 3.35 | 4.95 | −17.5 | 1.41 |
| | | sample 1 1 | | | | | | | |
| 4 | Zeta | pH 4.95 | 25 | −18.5 | −1.452 | 3.88 | | | |
| | | sample 1 2 | | | | | | | |
| 7 | Zeta | pH 7.07 | 25 | −31.8 | −2.496 | 2.16 | 7.07 | −32.7 | 1.27 |
| | | sample 1 1 | | | | | | | |
| 8 | Zeta | pH 7.07 | 25 | −33.6 | −2.637 | 2.4 | | | |
| | | sample 1 2 | | | | | | | |
| 9 | Zeta | pH 8.94 | 25 | −23.1 | −1.814 | 1.56 | 8.94 | −26.6 | 4.88 |
| | | sample 1 1 | | | | | | | |
| 10 | Zeta | pH 8.94 | 25 | −30 | −2.352 | 1.74 | | | |
| | | sample 1 2 | | | | | | | |
| 11 | Zeta | pH 11.21 | 25 | −5.92 | −0.4641 | 3.71 | 11.21 | −8.4 | 3.52 |
| | | sample 1 1 | | | | | | | |
| 12 | Zeta | pH 11.21 | 25 | −10.9 | −0.8535 | 4.35 | | | |
| | | sample 1 2 | | | | | | | |
| 13 | Zeta | pH 3.01 | 25 | 8.2 | 0.6428 | 6.69 | | | |
| | | sample 2 1 | | | | | | | |
| 14 | Zeta | pH 3.01 | 25 | 9.07 | 0.7111 | 6.82 | | | |
| | | sample 2 2 | | | | | | | |

TABLE 4
Protein X Exosomes

| | | | | | | | | | Std |
|---|---|---|---|---|---|---|---|---|---|
| | | Sample | T | ZP | Mobility | Cond | | Average | Dev |
| Record | Type | Name | ° C. | mV | μmcm/Vs | mS/cm | pH | (mV) | (mV) |
| 1 | Zeta | pH 3.15 | 25 | 6.87 | 0.5386 | 11.2 | | | |
| | | proteinX | | | | | | | |
| | | sample 1 1 | | | | | | | |
| 2 | Zeta | pH 3.15 | 25 | 6.36 | 0.4988 | 11.6 | | | |
| | | proteinX | | | | | | | |
| | | sample 1 2 | | | | | | | |
| 3 | Zeta | pH 2.95 | 25 | 10.4 | 0.8167 | 8.39 | 2.95 | 10.1 | 0.45 |
| | | proteinX | | | | | | | |
| | | sample 2 1 | | | | | | | |
| 4 | Zeta | pH 2.95 | 25 | 9.76 | 0.765 | 8.57 | | | |
| | | ProteinX | | | | | | | |
| | | sample 2 2 | | | | | | | |
| 7 | Zeta | pH 5.20 | 25 | −10.3 | −0.8057 | 8.98 | 5.2 | −10.8 | 0.64 |
| | | ProteinX | | | | | | | |
| | | sample 1 1 | | | | | | | |
| 8 | Zeta | pH 5.20 | 25 | −11.2 | −0.8793 | 9.41 | | | |
| | | ProteinX | | | | | | | |
| | | sample 1 2 | | | | | | | |
| 11 | Zeta | pH 6.95 | 25 | −13 | −1.022 | 5.8 | 6.95 | −13.8 | 1.06 |
| | | Protein X | | | | | | | |
| | | sample 1 1 | | | | | | | |
| 12 | Zeta | pH 6.95 | 25 | −14.5 | −1.135 | 5.91 | | | |
| | | Protein X | | | | | | | |
| | | sample 1 2 | | | | | | | |
| 15 | Zeta | pH 9.51 | 25 | −11.3 | −0.888 | 12.9 | 9.51 | −12.3 | 1.41 |
| | | Protein X | | | | | | | |
| | | sample 1 1 | | | | | | | |
| 16 | Zeta | pH 9.51 | 25 | −13.3 | −1.041 | 13.4 | | | |
| | | Protein X | | | | | | | |
| | | sample 1 2 | | | | | | | |
| 19 | Zeta | pH 11.08 | 25 | −15.9 | −1.243 | 6.26 | 11.08 | −17.0 | 1.56 |
| | | Protein X | | | | | | | |
| | | sample 1 1 | | | | | | | |
| 20 | Zeta | pH 11.08 | 25 | −18.1 | −1.416 | 6.57 | | | |
| | | Protein X | | | | | | | |
| | | sample 1 2 | | | | | | | |
| 33 | Zeta | pH 6.01 | 25 | −13.1 | −1.028 | 5.47 | 6.01 | −13.7 | 0.78 |
| | | Protein X | | | | | | | |
| | | sample 1 1 | | | | | | | |
| 34 | Zeta | pH 6.01 | 25 | −14.2 | −1.113 | 5.55 | | | |
| | | Protein X | | | | | | | |
| | | sample 1 2 | | | | | | | |
| 39 | Zeta | pH 6.38 | 25 | −13.4 | −1.051 | 5.49 | 6.38 | −14.3 | 1.20 |
| | | Protein X | | | | | | | |
| | | sample 1 1 | | | | | | | |
| 40 | Zeta | pH 6.38 | 25 | −15.1 | −1.182 | 5.57 | | | |
| | | Protein X | | | | | | | |
| | | sample 1 2 | | | | | | | |

These data were generated by dialyzing off the salts, then dispersing in the relevant buffer at the desired pH. It was observed that the presence of PBS protects the exosomes from aggregation over a broader pH range. The results shown in the above tables are presented as FIG. 2A.

TABLE 5
Average pH and Zeta Potential of Native and Protein X Exosomes

| Native | | PrX | |
|---|---|---|---|
| pH | Zeta | pH | Zeta |
| 3.01 | 9.5 | 2.95 | 10.1 |
| 4.95 | −17.5 | 5.2 | −10.8 |
| 7.07 | −32.7 | 6.95 | −13.8 |
| 8.94 | −26.6 | 9.51 | −12.3 |
| 11.21 | −8.4 | 11.08 | −17.0 |
| | | 6.01 | −13.7 |
| | | 6.38 | −14.3 |

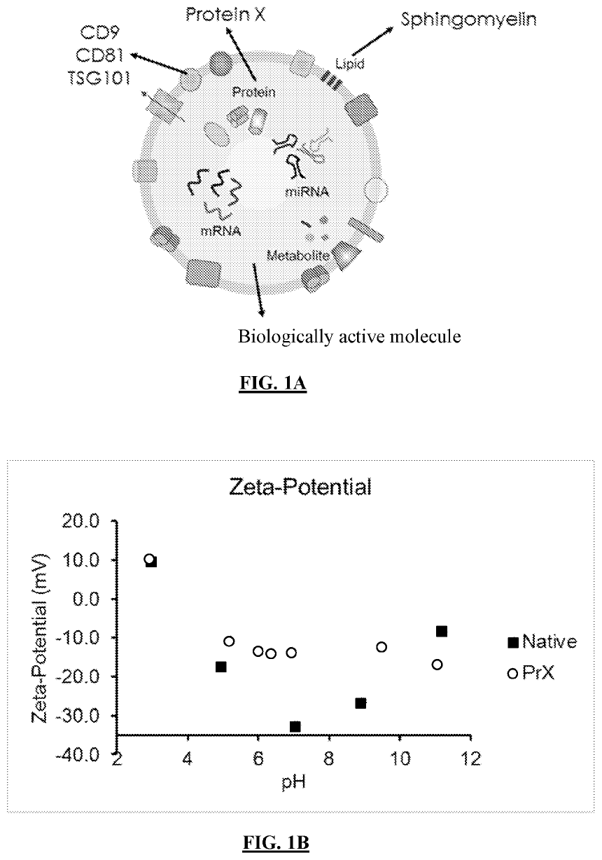

The results shown in Table 5 are also presented graphically as FIG. 1B.

### Example 2: Formulation of Buffer A

Exosomal Buffer A, for the storage and administration of EVs was prepared as described below. The EVs formulated for Buffer A is engineered to express a PTGFRN protein.

For pH adjustment, 50 mL of 1N sodium hydroxide and 50 mL of 1N hydrochloric acid were prepared. Water for injection (0.8 L) was added to a 1 L beaker with stir bar. While stirring using a stir plate (not heat) the following ingredients were added: (a) 50.00 g Sucrose, (b) 0.70 g Potassium Phosphate, Monobasic, (c) 4.00 g Sodium Phosphate, Dibasic, 7-Hydrate, and (d) 2.90 g Sodium Chloride. The pH was not adjusted until all of the powder had dissolved. If powder did not dissolve, the mixing speed was increased. Then, the pH was adjusted to between 7.1 and 7.3. If the pH was >7.3, 1N hydrochloric acid was used for adjustment. If the pH was <7.1, 1N sodium hydroxide was used for adjustment The solution was poured into a 1 L graduated cylinder. Water for injection was added to bring the total volume up to 1 L. The pH and conductivity was recorded. The target pH was between 7.1 and 7.3. The target conductivity is between 6.5+/−10% and 8.0+/−10% mS/cm. The solution was then filter sterilized through a 0.2 μm bottle top filter (cat #567-0020, Thermo Fisher Scientific, Waltham, MA). The final formulation for Buffer A is set forth in Table 6, below.

TABLE 6
Buffer A Formulation
Buffer A
Density: 1.02 kg/L

| | Concentration | | Concentration |
|---|---|---|---|
| Description | g/L | | mMol |
| Sucrose | 50 | g/L | 146.07 |
| Potassium Phosphate, Monobasic | 0.70 | g/L | 5.14 |
| Sodium Phosphate, Dibasic, 7-Hydrate | 4.00 | g/L | 14.92 |
| Sodium Chloride | 2.90 | g/L | 49.62 |

| | |
|---|---|
| WFI | As needed |

### Example 3: Formulation of Buffer B

Exosomal Buffer B, for the storage and administration of EVs was prepared as described below. The EVs formulated for Buffer B is engineered to express a PTGFRN protein and further comprise CL656.

For pH adjustment, 50 mL of 1N sodium hydroxide and 50 mL of 1N hydrochloric acid were prepared. Water for injection (0.8 L) was added to a 1 L beaker with stir bar. While stirring using a stir plate (not heat) the following ingredients were added: (a) 50.00 g Sucrose, (b) 2.10 g Potassium Phosphate, Monobasic, (c) 7.26 g Sodium Phosphate, Dibasic, 7-Hydrate, and (d) 2.34 g Sodium Chloride. The pH was not adjusted until all of the powder had dissolved. If powder did not dissolve, the mixing speed was increased. Then, the pH was adjusted to between 7.1 and 7.3. If the pH was >7.3, 1N hydrochloric acid was used for adjustment. If the pH was <7.1, 1N sodium hydroxide was used for adjustment The solution was poured into a 1 L graduated cylinder. Water for injection was added to bring the total volume up to 1 L. The pH and conductivity was recorded. The target pH was between 7.1 and 7.3. The target conductivity is between 6.5 and 8.0 mS/cm. The solution was then filter sterilized through a 0.2 μm bottle top filter (cat #567-0020, Thermo Fisher Scientific, Waltham, MA). The final formulation for Buffer B is set forth in Table 7, below.

TABLE 7
Buffer B Formulation
Buffer B
Density: 1.02 kg/L

| | Concentration | | Concentration |
|---|---|---|---|
| Description | g/L | | mMol |
| Sucrose | 50 | g/L | 146.07 |
| Potassium Phosphate, Monobasic | 2.10 | g/L | 15.44 |
| Sodium Phosphate, Dibasic, 7-Hydrate | 7.26 | g/L | 27.08 |
| Sodium Chloride | 2.34 | g/L | 40.04 |

| | |
|---|---|
| WFI | As needed |

### Example 4: Formulation of Buffer C

Exosomal Buffer C was prepared according to the formula set forth in Table 8, below. The EVs were engineered to express a PTGFRN protein fused to 11 L-12.

TABLE 8
Buffer C Formulation

| | Reagent | FW | mol/L | g/L |
|---|---|---|---|---|
| 0.032M Sodium | Sodium Phosphate | 268.07 | 0.0317 | 8.50 |
| Phosphate Dibasic, | dibasic | | | |
| 0.011M Potassium | heptahydrate | | | |
| Phosphate | Potassium | 136.09 | 0.0110 | 1.50 |
| Monobasic, 0.04M | Phosphate | | | |
| Sodium Chloride, | Monobasic | | | |
| 0.146M sucrose, | Sodium Chloride | 58.44 | 0.040 | 2.34 |
| pH 7.2 | Sucrose | 342.3 | 0.146 | 50 |

| | | |
|---|---|---|
| Density = | pH = | Conductivity = |
| 1.022 g/mL | 7.2 ± 0.2 | 8.7 ± 0.9 mS/cm |

### Example 5: Distribution of STING Agonist Concentration in Exosomes and Supernatants Over Time

The distribution of biologically active moieties, i.e., STING agonists, in exosomes and supernatants was investigated in an equilibration study.

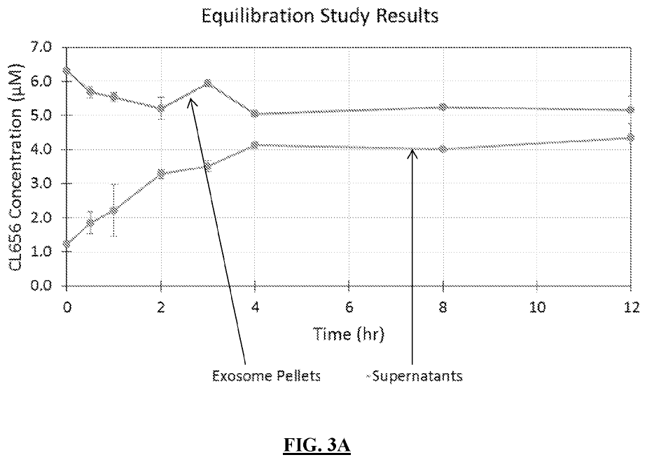

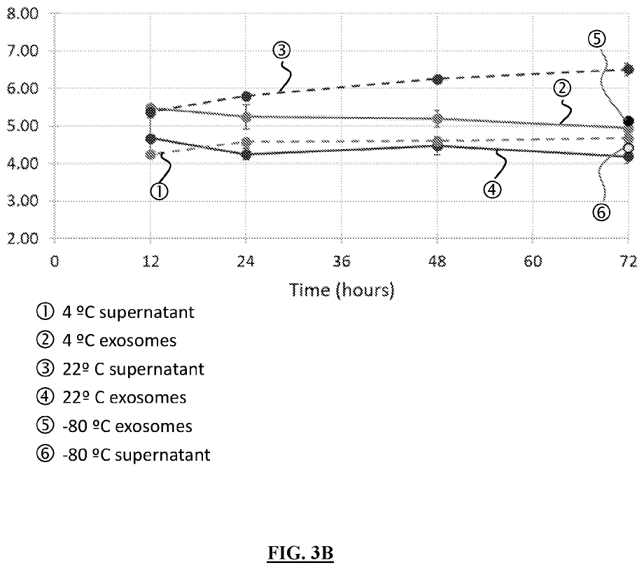

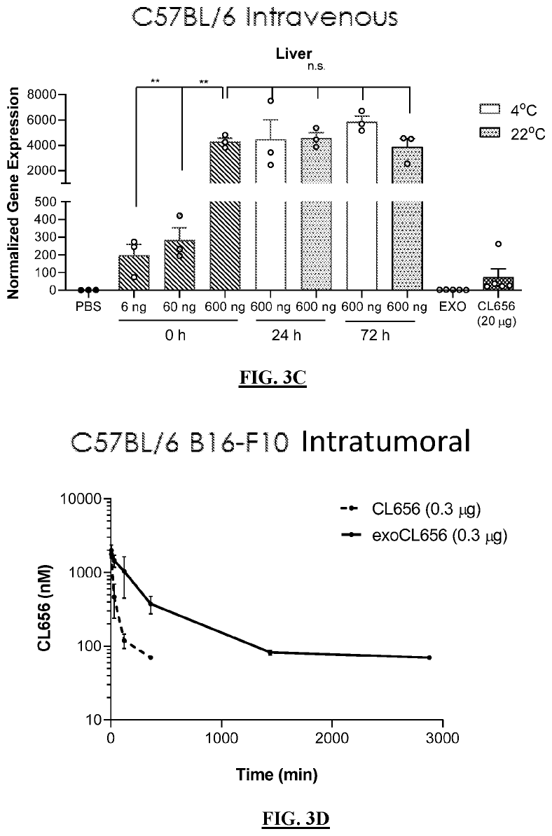

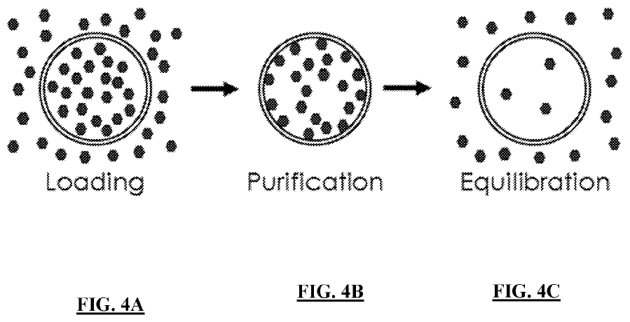

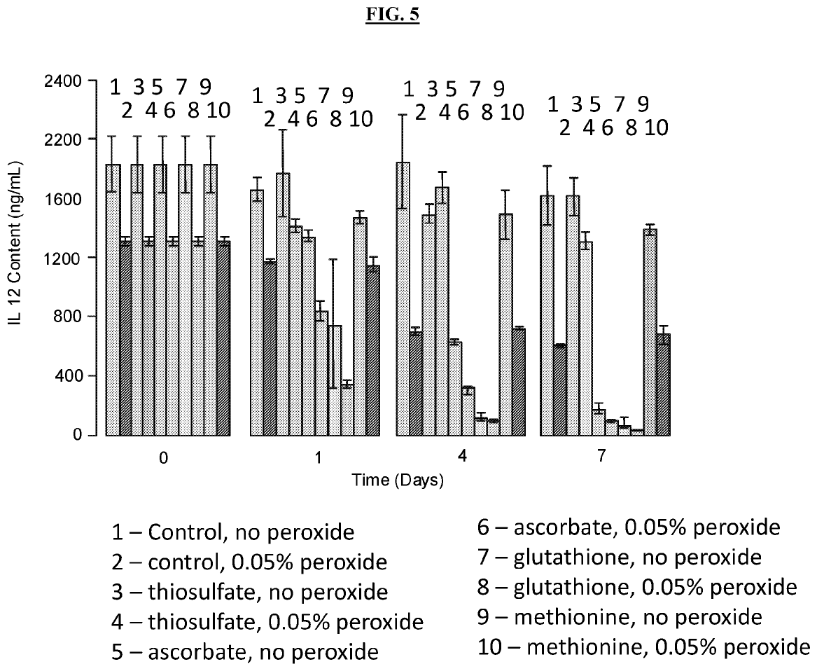

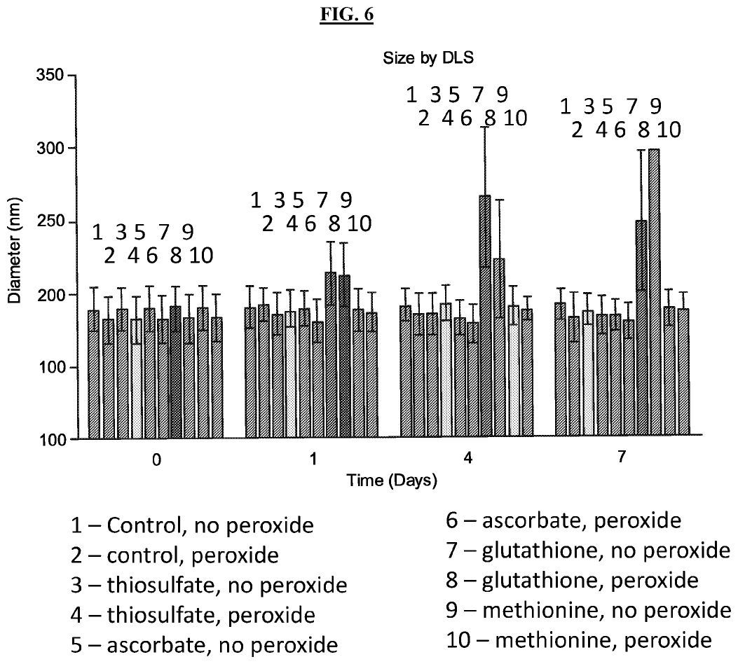

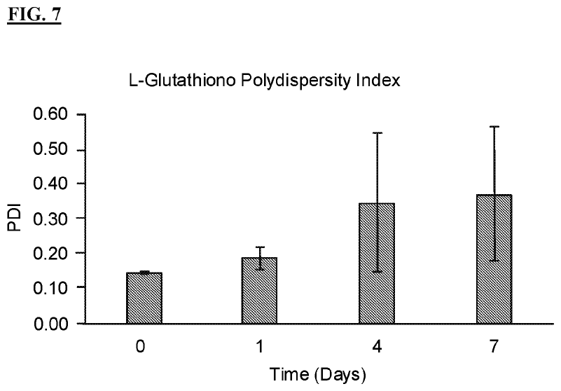

When present in exosomes, biologically active moieties (FIG. 1A), e.g., STING agonists, can passively diffuse out of exosomes and into the supernatant over time (FIGS. 4A-4C). The distribution of STING agonist in exosomes and supernatants was ascertained by calculating the concentration of STING agonist over time. Briefly, exosomes were loaded with STING agonists and stored in Buffer B. The exosome-containing liquid buffer formulations were either frozen at −80° C., semi-frozen at 4° C., or stored at 22° C., for a period of 72 hours. After 12 hours of storage in Buffer B at the temperatures described, the concentration of the STING agonists in exosomes was calculated and compared to the concentration of STING agonist in the supernatants. The results are set forth in FIGS. 3A and 3B. As shown, when stored at −80° C. for 72 hours, the final concentration of STING agonist in exosomes was about 5 μM and the final concentration of STING agonist in the supernatant was about 4.3 μM. When stored at 4° C. for 72 hours, the final concentration of STING agonist in exosomes was about 4.5 μM and the final concentration of STING agonist in the supernatant was about 4.4 μM. When stored at 22° C. for 72 hours, the final concentration of STING agonist in exosomes was about 4.1 μM and the final concentration of STING agonist in the supernatant was about 6.5 μM. The data graphed at FIGS. 3A and 3B are set forth in Tables 9, 10, and 11 below.

This example demonstrates that the storage temperature does not significantly affect the concentration of STING agonist in exosomes in comparison to the concentration of STING agonist in supernatants.

TABLE 9
Equilibrium Study Data

| Sample | | Time Point | Concentration |
|---|---|---|---|
| ID | Sample Type | (hr) | (μM) |
| 1 | Exosome Pellet | 0 | 6.35 |
| 2 | Exosome Pellet | 0 | 6.29 |
| 3 | Exosome Pellet | 0.5 | 5.56 |
| 4 | Exosome Pellet | 0.5 | 5.80 |
| 5 | Exosome Pellet | 1 | 5.44 |
| 6 | Exosome Pellet | 1 | 5.63 |
| 7 | Exosome Pellet | 2 | 5.44 |
| 8 | Exosome Pellet | 2 | 4.98 |
| 9 | Exosome Pellet | 3 | 5.87 |
| 10 | Exosome Pellet | 3 | 6.02 |
| 11 | Exosome Pellet | 4 | 4.99 |
| 12 | Exosome Pellet | 4 | 5.09 |
| 13 | Exosome Pellet | 8 | 5.29 |
| 14 | Exosome Pellet | 8 | 5.18 |
| 15 | Exosome Pellet | 12 | 4.87 |
| 16 | Exosome Pellet | 12 | 5.45 |
| 17 | Supernatant | 0 | 1.23 |
| 18 | Supernatant | 0 | 1.21 |
| 19 | Supernatant | 0.5 | 2.08 |
| 20 | Supernatant | 0.5 | 1.62 |
| 21 | Supernatant | 1 | 1.69 |
| 22 | Supernatant | 1 | 2.76 |
| 23 | Supernatant | 2 | 3.19 |
| 24 | Supernatant | 2 | 3.38 |
| 25 | Supernatant | 3 | 3.40 |
| 26 | Supernatant | 3 | 3.63 |
| 27 | Supernatant | 4 | 4.05 |
| 28 | Supernatant | 4 | 4.19 |
| 29 | Supernatant | 8 | 4.03 |
| 30 | Supernatant | 8 | 4.00 |
| 31 | Supernatant | 12 | 4.44 |
| 32 | Supernatant | 12 | 4.23 |

TABLE 10
Equilibrium Study Data - Exosome Pellets

Exosome Pellets

| Time Point | Concentration (μM) | Error (μM) |
|---|---|---|
| 0 | 6.32 | 0.04 |
| 0.5 | 5.68 | 0.17 |
| 1 | 5.53 | 0.13 |
| 2 | 5.21 | 0.32 |
| 3 | 5.94 | 0.11 |
| 4 | 5.04 | 0.07 |
| 8 | 5.24 | 0.08 |
| 12 | 5.16 | 0.41 |

TABLE 11
Equilibrium Study Data - Supernatants

Supernatants

| Time Point | Concentration (μM) | Error (μM) |
|---|---|---|
| 0 | 1.22 | 0.01 |
| 0.5 | 1.85 | 0.32 |
| 1 | 2.22 | 0.76 |
| 2 | 3.28 | 0.13 |
| 3 | 3.51 | 0.16 |
| 4 | 4.12 | 0.10 |
| 8 | 4.01 | 0.02 |
| 12 | 4.34 | 0.15 |

### Example 6: Effect of STING Agonist in Buffer B on Gene Expression

The effect of administration of exosomes containing STING agonist CL656 on gene expression in C57BL/6 liver was investigated.

After thawing, exosomes in buffer B composition containing STING agonist CL656 was stored at 4° C. and 22° C. for 24 h or 72 h as described in Example 5. PBS, CL656 (20 μg), exosomes, or exosomes in buffer B containing STING agonist CL656 that incubated as described above (600 ng of CL656 per mouse) were administered into C57BL/6 mice intravenously. As a control, exosomes in buffer B composition containing STING agonist CL656 that were stored at −80° C. (6 ng, 60 ng, and 600 ng per mouse) were administered (considered as 0 h). After 4 hours, mice were euthanized and liver was collected. Total RNA was isolated from liver by using RNeasy Lipid Mini Kit (Qiagen) and expression level of IFNβ mRNA was determined by RT-qPCR. Relative expression was normalized against housekeeping gene RPS13. FIG. 3C provides the results from the liver study. As shown, 1) dose dependent induction of IFNβ mRNA was observed at 0 h samples, 2) level of IFNβ mRNA was similar across all exosomes in buffer B composition containing STING agonist CL656 (600 ng per mouse injection) at any storage conditions, 3) exosomes without STING agonist did not induce the IFNβ mRNA and CL656 (20 μg) induced low level of IFNβ mRNA.

These results demonstrate that the administration of a STING agonist contained in Buffer B which had been frozen, thawed, and administered to subjects, is effective at inducing IFNβ gene expression in liver tissue without any significant differences.

### Example 7: Effect of STING Agonist and Exosome-Encapsulated STING Agonist on Intratumoral Concentration

The intratumoral concentration of STING agonist in Buffer B over time was investigated.

Briefly, B16-F10 melanoma cells were subcutaneously transplanted into C57BL/6 mice. When dermal tumors were visible, each animal received an intratumoral dose of either free STING agonist (0.3 μg of CL656) or exosome-encapsulated STING agonist (0.3 μg of CL656). Intratumoral concentration of free STING agonist and exosome-encapsulated STING agonist was measured at 5, 30, 120, 360, 1440, and 2880 minutes after injection. The results are provided in FIG. 3D. As shown, the intratumoral concentration of free STING agonist fell significantly after administration (dashed line), and was not detectable after about 360 minutes. Further shown in FIG. 3D, the concentration of exosome-encapsulated STING agonist fell slowly to about 100 nM at 1440 minutes. Thereafter, the concentration of exosome-encapsulated STING agonist was steady at about 100 nM until the last measurement was taken at 2880 minutes.

This example demonstrates that administration of a composition containing an exosome-encapsulated STING agonist, as compared to free STING agonist, has a stabilizing effect on intratumoral concentration of the STING agonist.

### Example 8: Exemplary Composition 02 Formulation Development

Formulation Development Overview

The STING-agonist-loaded exosome (exoSTING) Composition 02 (C-02) drug product formulation development occurred concurrently with formulation development of the intermediate-1 containing the purified exosomes. The objective for concurrent formulation development was: a) a recognition that the stability of the purified exosome active ingredient in the drug product was likely to inform the selection of stabilizing excipients as opposed to the stability of the agonist active ingredient and b) having the purified exosome formulation the same as the C-02 drug product would allow for ease in manufacturing since these excipients would be incorporated into the drug product. Test methods used for the studies were similar, although not identical to release test methods. Final qualified test methods were used for a final formulation stress conditions stability study. Studies were performed with C-02 drug product (containing intermediate-1 and intermediate-02) unless otherwise stated.

An initial drug product stress study was performed and included mechanical, oxidative and pH stress conditions. The study was performed using 15 mM potassium phosphate monobasic, 27 mM sodium phosphate dibasic buffer, 100 mM sodium chloride, 5% sucrose (w/v), pH 7.2. The study demonstrated acceptable stability of the drug product formulation for all but extreme mechanical stress such as vigorous agitation. The data indicated that PBS formulations containing 5% (w/v) sucrose provided protection to stress conditions.

A broad pH range study (pH 3.0 to 11.0), performed in different buffers using intermediate-01, identified the pH range between 6.5 and 9.0 as being acceptable (by dynamic light scattering (DLS)) and confirmed the suitability of phosphate buffer. A 9 month stability study for intermediate-01 using DLS as well as additional analytical assessments, confirmed stability at −80° C. in PBS containing 5% (w/v) sucrose.

The final formulation composition for both intermediate-01 and C-02 drug product was modified to achieve tonicity suitable for parenteral administration. Due to the desired sucrose levels and tonicity target, the sodium chloride concentration of the PBS formulation was reduced. To improve the buffering capacity of the formulation, the phosphate concentration of PBS was increased. Further, a mixture of sodium and potassium phosphate buffer was chosen over sodium phosphate alone.

A formulation composition selected was 15 mM potassium phosphate monobasic, 27 mM sodium phosphate dibasic, 40 mM sodium chloride, 5% (w/v) sucrose, pH 7.2. This formulation is used for intermediate-01, C-02 drug product, and FB-01 diluent for C-02. The final formulation buffer is referred to as formulation buffer 01 (FB-01).

An assessment of C-02 drug product stability in the final formulation demonstrates that drug product is stable to freeze-thaw cycling and mechanical inversion. Degradation occurs only under extreme stress conditions.

Evaluation of Cryoprotectants on Drug Product Freeze-Thaw Stability

Cryoprotectants, including sucrose, trehalose and D-sorbitol were evaluated at different concentrations in a drug product development lot formulated in 310 mM sodium phosphate dibasic, 90 mM potassium phosphate monobasic buffer, pH 7.4. This study used a development lot of PTGFRN-overexpressing exosomes and a cyclic dinucleotide STING agonist. The cryoprotectants were evaluated at 1.0, 2.5, 5.0, 7.5, and 10.0% (w/v). Theoretical osmolality for all of the sucrose and trehalose formulations in the phosphate buffer was calculated as approximately 300 mOsm/kg. For the D-sorbitol formulations, the osmolality of the 1% to 5% (w/v) formulations was also approximately 300 mOsm/kg while the 7.5 and 10.0% D-sorbitol formulations were calculated at approximately 400 and 600 mOsm/kg respectively. All formulations were evaluated after 0, 3, and 10 freeze-thaw cycles by appearance and size by dynamic light scattering (DLS).

The results for appearance testing for the two controls (PBS only or water) showed more substantial appearance changes associated with increasing freeze-thaw cycles compared to the formulations containing the cryoprotectants. The changes included increases in turbidity and color (PBS) or visible particulates with sedimentation (water). Both sucrose and D-sorbitol formulations showed acceptable appearance results at several concentrations. Higher concentrations of trehalose were required to prevent changes in appearance compared to sucrose or D-sorbitol.

Results for DLS testing showed that increases in size and polydispersity for the control formulations (PBS only, water only) were observed upon multiple freeze-thaw cycles. In general, no significant changes were observed in size or polydispersity after 3 and 10 freeze-thaws when higher concentrations of cryoprotectant (≥2.5% (w/v)) were used.

Based on the appearance and DLS results in this freeze-thaw study, both sucrose and D-sorbitol are acceptable cryoprotectants when present at ≥2.5% (w/v). 5% (w/v) sucrose provided sufficient cryoprotection and was chosen for future formulation studies, including in the initial stress studies described below.

Stress Studies in Phosphate Buffered Saline with Sucrose—Initial Study

Stress studies were performed using the selected cryoprotectant (5% (w/v) sucrose) and phosphate buffer containing sodium chloride to confirm stability under different conditions of mechanical stress and after oxidation. The formulation for these studies was 43 mM phosphate buffer, 100 mM sodium chloride, 5% (w/v) sucrose, pH 7.2.

For the mechanical stress study, samples were evaluated at ambient temperature using varying levels of mechanical stress (agitation, mixing) for different durations. Samples were evaluated by appearance, pH, osmolality, DLS, and nanoparticle tracking analysis (NTA). Size and polydispersity index did not change for any of the conditions tested relative to the control (no agitation). Only manual vigorous agitation resulted in appearance test changes; foaming was observed for this condition. Acceptable stability of the drug product formulation was observed for all conditions except extreme mechanical stress such as vigorous agitation. Stability of the formulation after multiple gentle inversions was also observed. The pH and osmolality of the test samples remained constant.

The effect of oxidative stress on drug product was evaluated by treatment of samples with different concentrations of hydrogen peroxide at 37° C. for 4 hours. After treatment, samples were quenched with 100 mM sodium ascorbate, and held at ambient temperature for 1 hour to neutralize the hydrogen peroxide prior to testing. This study used intermediate-01 loaded with STING agonist. Samples were analyzed by appearance, DLS, NTA, and for in vivo potency.

The drug product is stable and fully potent when subjected to oxidative stress under the conditions tested with a limited set of assays. There were no substantial changes observed at the different hydrogen peroxide concentration exposures and relative to the control (no hydrogen peroxide treatment) for appearance, size, or exosome concentration. Qualitatively, no substantial differences in potency in the spleen were observed as assessed by normalized gene expression. Although a trend of decreasing potency in the liver was observed with increasing hydrogen peroxide test concentration, substantial loss of potency in the ascorbic acid only group was also observed along with a relatively wide range of potency results inter- and intra-groups. Therefore, the potency was also similar in the liver between the test and control groups, except for the control group (PBS). The data suggest drug product is not sensitive to low levels of oxidative stress by the test methods employed.

A subsequent stress study was designed to further confirm stability of the C-02 drug product in the final formulation selected. In addition, an accelerated conditions study was performed for C-02 drug product in the final formulation (data not shown).

Selection of pH/Intermediate-01 Study

Selection of the pH target and range for drug product was focused on ensuring stability of the exosomes in the formulation, and therefore a study was conducted using intermediate-01. The study evaluated stability at pH values of 3, 5, 6, 6.5, 7, 9, and 11. Citric acid/sodium citrate dihydrate buffer was used to achieve pH values of 3 and 5. Potassium phosphate monobasic/sodium phosphate dibasic heptahydrate was used for pH values 6, 6.5, and 7, and carbonate buffer was used for pH values of 9 and 11. Samples were analyzed by DLS for changes in size or size distribution, and ζ-potential for surface charge. While the combination of these limited data support a pH range from 6.5 to 9.0, in practice, a narrower range (±0.5) was selected for the formulation around the pH 7.2 target. Based on the results, a target pH of 7.2 was selected based on acceptable DLS results and expected buffering capacity for phosphate buffer.

Intermediate-01 and C-02 Drug Product Formulation

The intermediate-01 pH study data supports the selection of a PBS formulation for intermediate-01. The cryoprotectant study for drug product identified 5% (w/v) sucrose for the formulation. A similar study for intermediate-01 also identified 5% (w/v) sucrose as a desired cryoprotectant (data not shown). The formulation composition for both intermediate-01 and drug product also maintains tonicity close to physiological fluids (approximately 300 mOsm/kg). Due to the desired sucrose levels and tonicity target, the salt concentration of the PBS formulation was reduced. To improve the buffering capacity of the formulation, the phosphate concentration of PBS was increased. Further, a mixture of sodium and potassium phosphate buffer was chosen.

Based on these considerations one formulation composition selected for C-02 drug product was 15 mM potassium phosphate monobasic, 27 mM sodium phosphate dibasic, 40 mM sodium chloride, 5% w/v sucrose, pH 7.2. The formulation is used for intermediate-01, C-02 drug product, and FB-01 diluent for C-02 drug product. The formulation buffer is referred to as FB-01.

Intermediate-01 Stability Study

A development stability study was performed to confirm the stability of intermediate-01 for long-term storage at −80° C. in the FB-01.

Stress Study in Final Drug Product Formulation—Follow-up Study

A follow-up stress study was performed in the final formulation composition to confirm stability of C-02 drug product under different conditions of mechanical stress and after oxidation. This study was performed using C-02 drug product in the final FB-01 formulation and was performed with additional analytical methods relative to the initial stress study.

The study included a hold control (at 25° C. for up to 24 hr), multiple freeze-thaw cycles (up to 10 cycles with freeze to −80° C. and thaw at ambient temperature), pH extremes (pH 5 and 9), oxidative stress (3% hydrogen peroxide exposure for up to 24 hr), and mechanical stress using mechanical end-over-end inversion for up to 24 hr.

The freeze-thaw cycle results show no change in product quality attributes through 10 freeze-thaw cycles.

### Example 9: Exemplary Composition 03 Formulation Development

The IL-12-loaded exosomes (exoIL-12) Composition 03 (C-03) drug product is composed of the active drug substance, exoIL-12, in a formulation buffer of 5 mM potassium phosphate monobasic, 15 mM sodium phosphate dibasic, 50 mM sodium chloride, 146 mM sucrose, at pH 7.2.

Formulation development for C-03 was guided by several principles: the drug product formulation is intended for use as an injectable solution; and that the formulation is optimized for frozen storage to minimize degradation from freeze/thaw operations. During the formulation development studies, initial analytical testing focused on the stability of exosomes in solution and potency. These assays included appearance, dynamic light scattering (DLS), IL-12 AlphaLISA, and the human embryonic kidney (HEK) cell-based reporter assay.

After purification, the exo-IL-12 formulation appears as a semi-transparent solution with no visible particulates. Color was determined by comparing exosome samples to European Pharmacopeia color standards in glass ampoules. exoIL-12 exosomes have been found to measure approximately 171 nm in diameter with a PDI value of <0.25.

Buffer and cryoprotectant investigations for C-03 led to the selection of a sodium/potassium phosphate buffer with 146 mM (5% w/v) sucrose, at pH 7.2 for CB-102 drug substance and C-03 drug product formulation to optimize quality and stability. A forced degradation study was performed to inform on potential stability-indicating assays.

Selection of Buffer

A pH stability study was conducted using at elevated temperatures (37° C. and 50° C.), with various buffers at pH 3, 5, 7, and 9.

Buffer-102 was initially buffer exchanged into milliQ water and adjusted to the desired pH by spiking in concentrated stocks of the appropriate buffer. After pH adjustment, samples were analyzed by appearance, dynamic light scattering (DLS) for changes in size and size distribution, and IL-12 AlphaLISA to quantitate free vs. total (free and exosome-associated), IL-12 content.

Selection of Cryoprotectant

A study was performed using exoIL-12 in phosphate buffer with and without 146 mM (5% w/v) sucrose, subjected to repeat freeze and thaw (F/T) cycles. The concentration of cryoprotectant was chosen to achieve physiological osmolality (approximately 290 mOsm/kg), with sufficient phosphate buffering capacity (concentration). ExoIL-12 in milliQ water (MQ) and PBS only were prepared as control samples and subjected to 3 freeze/thaw cycles (F/T). ExoIL-12 in phosphate buffer (as selected from a buffer selection study) with 146 mM sucrose was evaluated at 0, 1, 3, and 10 F/T. Collected samples were evaluated by appearance, DLS, and IL-12 AlphaLISA test methods.

Test samples containing sucrose remained stable with respect to size by DLS through 10 F/T cycles, measuring approximately 180 nm. Similarly, the distribution or polydispersity index (PDI), remained consistently low at 0.15 up to 3 F/T cycles, while increases to 0.2 after 10 F/T cycles. No color change or increase in turbidity were observed. Control exosomes dispersed in either milliQ water or PBS and subjected to 3 F/T cycles showed clear signs of aggregation and degradation. The milliQ control samples became turbid and changed to a slightly red/orange color, while the size of ≥800 nm by DLS clearly indicated aggregation. The size of PBS-only exosomes was slightly larger than test samples, measuring 187 nm and the PDI was similar to that of 10 F/T samples for the test groups containing sucrose. PBS-only exosomes displayed a noted change in turbidity by appearance indicating aggregation, but no change in color.

IL-12 AlphaLISA results indicate both associated and free IL-12 remain similar at approximately 2300 ng/mL and 1-3% IL-12, respectively, after 10 F/T in the presence of sucrose. A notable decrease in associated IL-12 was observed for both milliQ and PBS-only exosomes: decreasing 24% and 37%, respectively. The amount of free IL-12 increased slightly to 5% and 4%, respectively.

The osmolality of the final phosphate/sucrose buffer formulation is approximately 300 mOsm/kg.

Forced Degradation Study

A forced degradation study was conducted in accordance with ICH Q1A(R2) to assess drug product stability under stress conditions of freeze-thaw, pH extremes, oxidation, and mechanical stress.

Results of the freeze and thaw study generally showed no significant trends through ten F/T cycles. While the observed exosome size and PDI values were the largest after 10 F/T, the absolute differences were not significant enough to indicate a true impact on exosome stability. In addition, the other test results did not show significant trends up to 10 F/T. Conservatively, stability of C-03 is confirmed for up to five freeze and thaw cycles.

### Example 10: Exemplary Composition 04 Formulation Development

A drug product formulation for exosomes loaded with antisense oligomers will be developed. The C-03 drug product formulation will be used as the starting point. Phosphate buffer concentration (5 mM potassium phosphate monobasic and 15 mM sodium phosphate dibasic) and pH (pH 7.2) will remain unchanged relative to the C-03 drug product. Sodium chloride concentrations will tested at concentrations ranging from about 50 mM to about 150 mM, and sucrose concentrations will be tested at concentrations from about 2.5% to 5% (about 73 mM to about 146 mM). Conditions will be monitored according to the methods described in Examples 9 and 10, above. Various ASO-loaded exosome constructs will be analyzed, including exosomes loaded with ASOs targeting STAT6, as disclosed herein. In aqueous Tris EDTA buffer or pure water the ASO dissociates from the exosomes, and thus a combination of sodium chloride and sucrose is likely to play a role in retaining the loaded ASO.

It is to be appreciated that the Detailed Description section, and not the Summary and Abstract sections, is intended to be used to interpret the claims. The Summary and Abstract sections can set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

The claims in the instant application are different than those of the parent application or other related applications. The Applicant therefore rescinds any disclaimer of claim scope made in the parent application or any predecessor application in relation to the instant application. The Examiner is therefore advised that any such previous disclaimer and the cited references that it was made to avoid, can need to be revisited. Further, the Examiner is also reminded that any disclaimer made in the instant application should not be read into or against the parent application.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific aspects have been illustrated and described, the above specification is not restrictive. It will be appreciated that various changes can be made without departing from the spirit and scope of the disclosure(s).

1. A pharmaceutical composition comprising
(a) an extracellular vesicle;
(b) a saccharide;
(c) sodium chloride;
(d) a potassium phosphate; and
(e) a sodium phosphate,
wherein the composition is in a solution at a pH of 7.1 to 7.3.

2. The composition of claim 1, wherein the extracellular vesicle is an exosome.

3. The composition of claim 1, wherein the composition is capable of being stored for at least about 4 hours at a temperature of 4° C.

4-5. (canceled)

6. The composition of claim 1, wherein the composition has a pH of 7.2.

7. (canceled)

8. The composition of claim 1, wherein the composition has (i) reduced aggregates, (ii) improved stability of the EV, (iii) improved integrity of the EV architecture, and (iv) improved stability of engineered proteins contained on or in EVs.

9-10. (canceled)

11. The composition of claim 1, wherein the saccharide comprises lactose, glucose, sucrose, trehalose, and/or combinations thereof.

12-17. (canceled)

18. The composition of claim 1, wherein the composition has a conductivity between about 6 mS/cm+/−10% and about 10 mS/cm+/−10%.

19-21. (canceled)

22. The composition of claim 1, wherein the sodium chloride is present in the composition at a concentration of between about 10 mM and about 134 mM.

23-32. (canceled)

33. The composition of claim 1, wherein the potassium phosphate is present in the composition at a concentration of between about 1 mM to about 20 mM, between about 2 mM to about 19 mM, between about 3 mM to about 18 mM, between about 4 mM to about 17 mM, between about 5 mM to about 16 mM, or between about 5 mM to about 15 mM.

34-37. (canceled)

38. The composition of claim 1, wherein the potassium phosphate is potassium phosphate monobasic.

39. The composition of claim 1, wherein the sodium phosphate is present in the composition at a concentration of between about 10 mM to about 30, between about 11 mM to about 29 mM, between about 12 mM to about 28 mM, between about 13 mM to about 27 mM, or between about 14 mM to about 26 mM.

40-44. (canceled)

45. The composition of any one of claims 1 to 44, further comprising an anti-oxidant, wherein the anti-oxidant comprises D-methionine, L-methionine, ascorbic acid, erythorbic acid, Na ascorbate, thioglycerol, cysteine, acetylcysteine, cystine, dithioerythreitol, glutathione, tocopherols, butylated hydroxyanisole (BHA), butylated hydroxytoluene (BHT), sodium bisulphate, sodium dithionite, A-Tocopherol, γ-Tocopherol, propyl gallate, ascorbyl palmitate, sodium metabisulfite, thiourea, sodium thiosulfate, propyl gallate, and sodium thioglycolate.

46. (canceled)

47. The composition of claim 1, wherein the composition:
(i) is not lyophilized,
(ii) does not comprise a chelating agent,
(iii) does not comprise albumin, or
(iv) any combination of (i) to (iii).

48-49. (canceled)

50. The composition of claim 1, comprising
(1)
a. a sucrose at a concentration of about 2.5% to about 5% w/v,
b. sodium chloride at a concentration of about 50 mM to 150 mM,
c. a potassium phosphate monobasic at a concentration of about 5 mM, and
d. a sodium phosphate dibasic heptahydrate at a concentration of about 15 mM;
wherein the composition is in a solution at a pH of 7.2 and at a conductivity of 7.23 mS/cm+/−10%; or
(2)
a. a sucrose at a concentration of about 5% w/v,
b. sodium chloride at a concentration of about 40 mM,
c. a potassium phosphate monobasic at a concentration of about 15 mM, and
d. a sodium phosphate dibasic heptahydrate at a concentration of about 27 mM,
wherein the composition is in a solution at a pH of 7.2 and at a conductivity of 8.8 mS/cm+/−10%.

51-75. (canceled)

76. A method of preparing the pharmaceutical composition of claim 1 comprising combining:
(a) an extracellular vesicle;
(b) a saccharide;
(c) sodium chloride;
(d) a potassium phosphate; and
(e) a sodium phosphate.

77-82. (canceled)

83. A method of treating a disease or a condition in a subject in need thereof comprising administering to the subject the composition of claim 1.

84-89. (canceled)

90. The composition of claim 1, wherein the extracellular vesicle comprises:
(i) an antisense oligonucleotide (ASO),
(ii) a STING agonist, or
(iii) an IL-12 moiety.

91. The composition of claim 1, comprising:
(a) Extracellular vesicles comprising an ASO, wherein the ASO comprises a nucleic acid sequence selected from SEQ ID NOs: 91-193;
(b) Sucrose at a concentration of 2.5% to about 5%;
(c) Sodium chloride at a concentration of 50 mM to 150 mM;
(d) Potassium phosphate monobasic at a concentration of 5 mM; and
(e) Sodium phosphate dibasic at a concentration of 15 mM.

92-93. (canceled)

94. The composition of claim 1, comprising:
(a) Extracellular vesicles;
(b) Sucrose at a concentration of about 5%;
(c) Sodium chloride at a concentration of about 50 mM;
(d) Potassium phosphate monobasic at a concentration of about 5 mM;
(e) Sodium phosphate dibasic at a concentration of about 15 mM, and
(f) wherein the pH of the composition is about 7.2;
wherein the extracellular vesicles comprise an IL-12 moiety.

95-115. (canceled)

116. The composition of claim 91, wherein
(i) the ASO comprises the nucleic acid sequence set forth in SEQ ID NO: 144, 145, 193, or 185;
(ii) the ASO is associated with the extracellular vesicles by a linker, comprising cholesterol, tocopherol, a fatty acid, or any combination thereof; or
(iii) any combination of (i) and (ii).


SEQUENCE LISTING (193 sequences)

SEQ ID NO: 1 - protein, 879 aa, Homo sapiens
MGRLASRPLLLALLSLALCRGRVVRVPTATLVRVVGTELVIPCNVSDYDGPSEQNFDWSFSSLGSSFVELASTWEVGFPAQLYQERLQRGEILLRRTANDAVELHIKNVQPSDQGHYKCSTPSTDATVQGNYEDTVQVKVLADSLHVGPSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 2 - protein, 731 aa, Artificial Sequence
  PTGFRN Protein Fragment #1
PSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 3 - protein, 611 aa, Artificial Sequence
  PTGFRN Protein Fragment #2
VATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 4 - protein, 485 aa, Artificial Sequence
  PTGFRN Protein Fragment #3
SPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 5 - protein, 343 aa, Artificial Sequence
  PTGFRN Protein Fragment #4
KPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 6 - protein, 217 aa, Artificial Sequence
  PTGFRN Protein Fragment #5
VRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 7 - protein, 66 aa, Artificial Sequence
  PTGFRN Protein Fragment #6
SKPVFITVKMDVLNAFKYPLLIGVGLSTVIGLLSCLIGYCSSHWCCKKEVQETRRERRRLMSMEMD

SEQ ID NO: 8 - protein, 21 aa, Artificial Sequence
  PTGFRN Protein - Signal Peptide
MGRLASRPLLLALLSLALCRG

SEQ ID NO: 9 - protein, 193 aa, Artificial Sequence
  PTGFRN Protein Fragment #7
GPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 10-22 - skipped (13 entries)

SEQ ID NO: 23 - DNA, 3963 bp, Artificial Sequence
  STAT6
ggggcagccactgcttacactgaagagggaggacgggagaggagtgtgtgtgtgtgtgtgtgtgtgtgtgtgtatgtatgtgtgtgctttatcttatttttctttttggtggtggtggtggaaggggggaggtgctagcagggccagccttgaactcgctggacagagctacagacctatggggcctggaagtgcccgctgagaaagggagaagacagcagaggggttgccgaggcaacctccaagtcccagatcatgtctctgtggggtctggtctccaagatgcccccagaaaaagtgcagcggctctatgtcgactttccccaacacctgcggcatcttctgggtgactggctggagagccagccctgggagttcctggtcggctccgacgccttctgctgcaacttggctagtgccctactttcagacactgtccagcaccttcaggcctcggtgggagagcagggggaggggagcaccatcttgcaacacatcagcacccttgagagcatatatcagagggaccccctgaagctggtggccactttcagacaaatacttcaaggagagaaaaaagctgttatggaacagttccgccacttgccaatgcctttccactggaagcaggaagaactcaagtttaagacaggcttgcggaggctgcagcaccgagtaggggagatccaccttctccgagaagccctgcagaagggggctgaggctggccaagtgtctctgcacagcttgatagaaactcctgctaatgggactgggccaagtgaggccctggccatgctactgcaggagaccactggagagctagaggcagccaaagccctagtgctgaagaggatccagatttggaaacggcagcagcagctggcagggaatggcgcaccgtttgaggagagcctggccccactccaggagaggtgtgaaagcctggtggacatttattcccagctacagcaggaggtaggggcggctggtggggagcttgagcccaagacccgggcatcgctgactggccggctggatgaagtcctgagaaccctcgtcaccagttgcttcctggtggagaagcagcccccccaggtactgaagactcagaccaagttccaggctggagttcgattcctgttgggcttgaggttcctgggggccccagccaagcctccgctggtcagggccgacatggtgacagagaagcaggcgcgggagctgagtgtgcctcagggtcctggggctggagcagaaagcactggagaaatcatcaacaacactgtgcccttggagaacagcattcctgggaactgctgctctgccctgttcaagaacctgcttctcaagaagatcaagcggtgtgagcggaagggcactgagtctgtcacagaggagaagtgcgctgtgctcttctctgccagcttcacacttggccccggcaaactccccatccagctccaggccctgtctctgcccctggtggtcatcgtccatggcaaccaagacaacaatgccaaagccactatcctgtgggacaatgccttctctgagatggaccgcgtgccctttgtggtggctgagcgggtgccctgggagaagatgtgtgaaactctgaacctgaagttcatggctgaggtggggaccaaccgggggctgctcccagagcacttcctcttcctggcccagaagatcttcaatgacaacagcctcagtatggaggccttccagcaccgttctgtgtcctggtcgcagttcaacaaggagatcctgctgggccgtggcttcaccttttggcagtggtttgatggtgtcctggacctcaccaaacgctgtctccggagctactggtctgaccggctgatcattggcttcatcagcaaacagtacgttactagccttcttctcaatgagcccgacggaacctttctcctccgcttcagcgactcagagattgggggcatcaccattgcccatgtcatccggggccaggatggctctccacagatagagaacatccagccattctctgccaaagacctgtccattcgctcactgggggaccgaatccgggatcttgctcagctcaaaaatctctatcccaagaagcccaaggatgaggctttccggagccactacaagcctgaacagatgggtaaggatggcaggggttatgtcccagctaccatcaagatgaccgtggaaagggaccaaccacttcctaccccagagctccagatgcctaccatggtgccttcttatgaccttggaatggcccctgattcctccatgagcatgcagcttggcccagatatggtgccccaggtgtacccaccacactctcactccatccccccgtatcaaggcctctccccagaagaatcagtcaacgtgttgtcagccttccaggagcctcacctgcagatgccccccagcctgggccagatgagcctgccctttgaccagcctcacccccagggcctgctgccgtgccagcctcaggagcatgctgtgtccagccctgaccccctgctctgctcagatgtgaccatggtggaagacagctgcctgagccagccagtgacagcgtttcctcagggcacttggattggtgaagacatattccctcctctgctgcctcccactgaacaggacctcactaagcttctcctggaggggcaaggggagtcggggggagggtccttgggggcacagcccctcctgcagccctcccactatgggcaatctgggatctcaatgtcccacatggacctaagggccaaccccagttggtgatcccagctggagggagaacccaaagagacagctcttctactacccccacagacctgctctggacacttgctcatgccctgccaagcagcagatggggagggtgccctcctatccccacctactcctgggtcaggaggaaaagactaacaggagaatgcacagtgggtggagccaatccactccttcctttctatcattcccctgcccacctccttccagcactgactggaagggaagttcaggctctgagacacaccccaacatgcctgcacctgcagcgcgcacacgcacgcacacacacatacagagctctctgagggtgatggggctgagcaggaggggggctgggtaagagcacaggttagggcatggaaggcttctccgcccattctgacccagggcctaggacggataggcaggaacatacagacacatttacactagaggccagggatagaggatattgggtctcagccctaggggaatgggaagcagctcaagggaccctgggtgggagcataggaggggtctggacatgtggttactagtacaggttttgccctgattaaaaaatctcccaaagccccaaattcctgttagccaggtggaggcttctgatacgtgtatgagactatgcaaaagtacaagggctgagattcttcgtgtatagctgtgtgaacgtgtatgtacctaggatatgttaaatgtatagctggcaccttagttgcatgaccacatagaacatgtgtctatctgcttttgcctacgtgacaacacaaatttgggagggtgagacactgcacagaagacagcagcaagtgtgctggcctctctgacatatgctaacccccaaatactctgaatttggagtctgactgtgcccaagtgggtccaagtggctgtgacatctacgtatggctccacacctccaatgctgcctgggagccagggtgagagtctgggtccaggcctggccatgtggccctccagtgtatgagagggccctgcctgctgcatcttttctgttgccccatccaccgccagcttcccttcactcccctatcccattctccctctcaaggcaggggtcatagatcctaagccataaaataaattttattccaaaataacaaaataaataatctactgtacacaatctgaaaa

SEQ ID NO: 24-88 - skipped (65 entries)

SEQ ID NO: 89 - DNA, 20 bp, Artificial Sequence
  Antisense Oligonucleotide
tgagcgaatggacaggtctt

SEQ ID NO: 90 - skipped

SEQ ID NO: 91 - DNA, 15 bp, Artificial Sequence
  Synthetic Peptide
ctggtgacgagggtt

SEQ ID NO: 92 - DNA, 15 bp, Artificial Sequence
  Synthetic Peptide
cgctcacaccgcttg

SEQ ID NO: 93 - DNA, 15 bp, Artificial Sequence
  Synthetic Peptide
aggctagtaacgtac

SEQ ID NO: 94 - DNA, 15 bp, Artificial Sequence
  Synthetic Peptide
gaaggctagtaacgt

SEQ ID NO: 95 - DNA, 15 bp, Artificial Sequence
  Synthetic Peptide
ggttccgtcgggctc

SEQ ID NO: 96 - DNA, 15 bp, Artificial Sequence
  Synthetic Peptide
aggttccgtcgggct

SEQ ID NO: 97 - DNA, 15 bp, Artificial Sequence
  Synthetic Peptide
aaggttccgtcgggc

SEQ ID NO: 98 - DNA, 15 bp, Artificial Sequence
  Synthetic Peptide
aaaggttccgtcggg

SEQ ID NO: 99 - DNA, 15 bp, Artificial Sequence
  Synthetic Peptide
gaaaggttccgtcgg

SEQ ID NO: 100 - DNA, 15 bp, Artificial Sequence
  Synthetic Peptide
agaaaggttccgtcg

SEQ ID NO: 101 - DNA, 15 bp, Artificial Sequence
  Synthetic Peptide
agtcgctgaagcgga

SEQ ID NO: 102 - DNA, 15 bp, Artificial Sequence
  Synthetic Peptide
gagtcgctgaagcgg

SEQ ID NO: 103 - DNA, 15 bp, Artificial Sequence
  Synthetic Peptide
cggattcggtccccc

SEQ ID NO: 104 - DNA, 15 bp, Artificial Sequence
  Synthetic Peptide
ccggattcggtcccc

SEQ ID NO: 105 - DNA, 15 bp, Artificial Sequence
  Synthetic Peptide
cccggattcggtccc

SEQ ID NO: 106 - DNA, 15 bp, Artificial Sequence
  Synthetic Peptide
tcccggattcggtcc

SEQ ID NO: 107 - DNA, 15 bp, Artificial Sequence
  Synthetic Peptide
gatcccggattcggt

SEQ ID NO: 108 - DNA, 15 bp, Artificial Sequence
  Synthetic Peptide
agatcccggattcgg

SEQ ID NO: 109 - DNA, 15 bp, Artificial Sequence
  Synthetic Peptide
aagatcccggattcg

SEQ ID NO: 110 - DNA, 15 bp, Artificial Sequence
  Synthetic Peptide
tgatacggggggatg

SEQ ID NO: 111 - DNA, 15 bp, Artificial Sequence
  Synthetic Peptide
cgtgtgcgcgctgca

SEQ ID NO: 112 - DNA, 16 bp, Artificial Sequence
  Synthetic Peptide
actggtgacgagggtt

SEQ ID NO: 113 - DNA, 16 bp, Artificial Sequence
  Synthetic Peptide
aactggtgacgagggt

SEQ ID NO: 114 - DNA, 16 bp, Artificial Sequence
  Synthetic Peptide
ctcacaccgcttgatc

SEQ ID NO: 115 - DNA, 16 bp, Artificial Sequence
  Synthetic Peptide
cggtcagaccagtagc

SEQ ID NO: 116 - DNA, 16 bp, Artificial Sequence
  Synthetic Peptide
ctagtaacgtactgtt

SEQ ID NO: 117 - DNA, 16 bp, Artificial Sequence
  Synthetic Peptide
gctagtaacgtactgt

SEQ ID NO: 118 - DNA, 16 bp, Artificial Sequence
  Synthetic Peptide
ggctagtaacgtactg

SEQ ID NO: 119 - DNA, 16 bp, Artificial Sequence
  Synthetic Peptide
aggctagtaacgtact

SEQ ID NO: 120 - DNA, 16 bp, Artificial Sequence
  Synthetic Peptide
aaggctagtaacgtac

SEQ ID NO: 121 - DNA, 16 bp, Artificial Sequence
  Synthetic Peptide
aagaaggctagtaacg

SEQ ID NO: 122 - DNA, 16 bp, Artificial Sequence
  Synthetic Peptide
aaaggttccgtcgggc

SEQ ID NO: 123 - DNA, 16 bp, Artificial Sequence
  Synthetic Peptide
agaaaggttccgtcgg

SEQ ID NO: 124 - DNA, 16 bp, Artificial Sequence
  Synthetic Peptide
ttcggtcccccagtga

SEQ ID NO: 125 - DNA, 16 bp, Artificial Sequence
  Synthetic Peptide
cggattcggtccccca

SEQ ID NO: 126 - DNA, 16 bp, Artificial Sequence
  Synthetic Peptide
agatcccggattcggt

SEQ ID NO: 127 - DNA, 16 bp, Artificial Sequence
  Synthetic Peptide
agcaagatcccggatt

SEQ ID NO: 128 - DNA, 16 bp, Artificial Sequence
  Synthetic Peptide
tgatacggggggatgg

SEQ ID NO: 129 - DNA, 16 bp, Artificial Sequence
  Synthetic Peptide
ttgatacggggggatg

SEQ ID NO: 130 - DNA, 16 bp, Artificial Sequence
  Synthetic Peptide
agaggccttgatacgg

SEQ ID NO: 131 - DNA, 16 bp, Artificial Sequence
  Synthetic Peptide
gggttagcatatgtca

SEQ ID NO: 132 - DNA, 17 bp, Artificial Sequence
  Synthetic Peptide
tggatctcccctactcg

SEQ ID NO: 133 - DNA, 17 bp, Artificial Sequence
  Synthetic Peptide
actggtgacgagggttc

SEQ ID NO: 134 - DNA, 17 bp, Artificial Sequence
  Synthetic Peptide
gctcacaccgcttgatc

SEQ ID NO: 135 - DNA, 17 bp, Artificial Sequence
  Synthetic Peptide
cgctcacaccgcttgat

SEQ ID NO: 136 - DNA, 17 bp, Artificial Sequence
  Synthetic Peptide
tccgctcacaccgcttg

SEQ ID NO: 137 - DNA, 17 bp, Artificial Sequence
  Synthetic Peptide
ttccgctcacaccgctt

SEQ ID NO: 138 - DNA, 17 bp, Artificial Sequence
  Synthetic Peptide
accagtagctccggaga

SEQ ID NO: 139 - DNA, 17 bp, Artificial Sequence
  Synthetic Peptide
gccggtcagaccagtag

SEQ ID NO: 140 - DNA, 17 bp, Artificial Sequence
  Synthetic Peptide
agccggtcagaccagta

SEQ ID NO: 141 - DNA, 17 bp, Artificial Sequence
  Synthetic Peptide
agaaggctagtaacgta

SEQ ID NO: 142 - DNA, 17 bp, Artificial Sequence
  Synthetic Peptide
aaggttccgtcgggctc

SEQ ID NO: 143 - DNA, 17 bp, Artificial Sequence
  Synthetic Peptide
aaaggttccgtcgggct

SEQ ID NO: 144 - DNA, 17 bp, Artificial Sequence
  Synthetic Peptide
gaaaggttccgtcgggc

SEQ ID NO: 145 - DNA, 17 bp, Artificial Sequence
  Synthetic Peptide
ctgagtcgctgaagcgg

SEQ ID NO: 146 - DNA, 17 bp, Artificial Sequence
  Synthetic Peptide
tctgagtcgctgaagcg

SEQ ID NO: 147 - DNA, 17 bp, Artificial Sequence
  Synthetic Peptide
atcccggattcggtccc

SEQ ID NO: 148 - DNA, 17 bp, Artificial Sequence
  Synthetic Peptide
gatcccggattcggtcc

SEQ ID NO: 149 - DNA, 17 bp, Artificial Sequence
  Synthetic Peptide
agatcccggattcggtc

SEQ ID NO: 150 - DNA, 17 bp, Artificial Sequence
  Synthetic Peptide
aagatcccggattcggt

SEQ ID NO: 151 - DNA, 17 bp, Artificial Sequence
  Synthetic Peptide
gcaagatcccggattcg

SEQ ID NO: 152 - DNA, 17 bp, Artificial Sequence
  Synthetic Peptide
cggtcatcttgatggta

SEQ ID NO: 153 - DNA, 17 bp, Artificial Sequence
  Synthetic Peptide
tgatacggggggatgga

SEQ ID NO: 154 - DNA, 17 bp, Artificial Sequence
  Synthetic Peptide
ttgatacggggggatgg

SEQ ID NO: 155 - DNA, 17 bp, Artificial Sequence
  Synthetic Peptide
cttgatacggggggatg

SEQ ID NO: 156 - DNA, 17 bp, Artificial Sequence
  Synthetic Peptide
gaggccttgatacgggg

SEQ ID NO: 157 - DNA, 20 bp, Artificial Sequence
  Synthetic Peptide
gggggtccctctgatatatg

SEQ ID NO: 158 - DNA, 20 bp, Artificial Sequence
  Synthetic Peptide
gtggatctcccctactcggt

SEQ ID NO: 159 - DNA, 20 bp, Artificial Sequence
  Synthetic Peptide
agcccaacaggaatcgaact

SEQ ID NO: 160 - DNA, 20 bp, Artificial Sequence
  Synthetic Peptide
cgctcacaccgcttgatctt

SEQ ID NO: 161 - DNA, 20 bp, Artificial Sequence
  Synthetic Peptide
ccgctcacaccgcttgatct

SEQ ID NO: 162 - DNA, 20 bp, Artificial Sequence
  Synthetic Peptide
tccgctcacaccgcttgatc

SEQ ID NO: 163 - DNA, 20 bp, Artificial Sequence
  Synthetic Peptide
agtttgccggggccaagtgt

SEQ ID NO: 164 - DNA, 20 bp, Artificial Sequence
  Synthetic Peptide
aagggcacgcggtccatctc

SEQ ID NO: 165 - DNA, 20 bp, Artificial Sequence
  Synthetic Peptide
aaagggcacgcggtccatct

SEQ ID NO: 166 - DNA, 20 bp, Artificial Sequence
  Synthetic Peptide
caaagggcacgcggtccatc

SEQ ID NO: 167 - DNA, 20 bp, Artificial Sequence
  Synthetic Peptide
acaaagggcacgcggtccat

SEQ ID NO: 168 - DNA, 20 bp, Artificial Sequence
  Synthetic Peptide
ccggagacagcgtttggtga

SEQ ID NO: 169 - DNA, 20 bp, Artificial Sequence
  Synthetic Peptide
tccggagacagcgtttggtg

SEQ ID NO: 170 - DNA, 20 bp, Artificial Sequence
  Synthetic Peptide
agtagctccggagacagcgt

SEQ ID NO: 171 - DNA, 20 bp, Artificial Sequence
  Synthetic Peptide
cggtcagaccagtagctccg

SEQ ID NO: 172 - DNA, 20 bp, Artificial Sequence
  Synthetic Peptide
agccggtcagaccagtagct

SEQ ID NO: 173 - DNA, 20 bp, Artificial Sequence
  Synthetic Peptide
cagccggtcagaccagtagc

SEQ ID NO: 174 - DNA, 20 bp, Artificial Sequence
  Synthetic Peptide
gctagtaacgtactgtttgc

SEQ ID NO: 175 - DNA, 20 bp, Artificial Sequence
  Synthetic Peptide
aagaaggctagtaacgtact

SEQ ID NO: 176 - DNA, 20 bp, Artificial Sequence
  Synthetic Peptide
gaagaaggctagtaacgtac

SEQ ID NO: 177 - DNA, 20 bp, Artificial Sequence
  Synthetic Peptide
agaagaaggctagtaacgta

SEQ ID NO: 178 - DNA, 20 bp, Artificial Sequence
  Synthetic Peptide
gagaaaggttccgtcgggct

SEQ ID NO: 179 - DNA, 20 bp, Artificial Sequence
  Synthetic Peptide
ggagaaaggttccgtcgggc

SEQ ID NO: 180 - DNA, 20 bp, Artificial Sequence
  Synthetic Peptide
cggattcggtcccccagtga

SEQ ID NO: 181 - DNA, 20 bp, Artificial Sequence
  Synthetic Peptide
ccggattcggtcccccagtg

SEQ ID NO: 182 - DNA, 20 bp, Artificial Sequence
  Synthetic Peptide
atcccggattcggtccccca

SEQ ID NO: 183 - DNA, 20 bp, Artificial Sequence
  Synthetic Peptide
agatcccggattcggtcccc

SEQ ID NO: 184 - DNA, 20 bp, Artificial Sequence
  Synthetic Peptide
aagatcccggattcggtccc

SEQ ID NO: 185 - DNA, 20 bp, Artificial Sequence
  Synthetic Peptide
gcaagatcccggattcggtc

SEQ ID NO: 186 - DNA, 20 bp, Artificial Sequence
  Synthetic Peptide
tgagcaagatcccggattcg

SEQ ID NO: 187 - DNA, 20 bp, Artificial Sequence
  Synthetic Peptide
tgatacggggggatggagtg

SEQ ID NO: 188 - DNA, 20 bp, Artificial Sequence
  Synthetic Peptide
ttgatacggggggatggagt

SEQ ID NO: 189 - DNA, 20 bp, Artificial Sequence
  Synthetic Peptide
gggagaggccttgatacggg

SEQ ID NO: 190 - DNA, 20 bp, Artificial Sequence
  Synthetic Peptide
gatcaccaactggggttggc

SEQ ID NO: 191 - DNA, 20 bp, Artificial Sequence
  Synthetic Peptide
tgcgtgtgcgcgctgcaggt

SEQ ID NO: 192 - DNA, 20 bp, Artificial Sequence
  Synthetic Peptide
gtgcgtgtgcgcgctgcagg

SEQ ID NO: 193 - DNA, 20 bp, Artificial Sequence
  Synthetic Peptide
gcccttgtacttttgcatag